(19) United States
(12) Patent Application Publication
(10) Pub. No.: US 2014/0024591 A1
(43) Pub. Date: Jan. 23, 2014

(54) Methods For Characterizing And Isolating Circulating Tumor Cell Subpopulations

(71) Applicants: Sabine Mai, Winnipeg (CA); Yvon E. Cayre, Paris (FR); Janine Wechsler, Paris (FR)

(72) Inventors: Sabine Mai, Winnipeg (CA); Yvon E. Cayre, Paris (FR); Janine Wechsler, Paris (FR)

(21) Appl. No.: 13/869,797

(22) Filed: Apr. 24, 2013

Related U.S. Application Data
(60) Provisional application No. 61/637,692, filed on Apr. 24, 2012.

(30) Foreign Application Priority Data
Apr. 24, 2012 (CA) 2775315

(51) Int. Cl. C12Q 1/68

(52) U.S. Cl. CPC C12Q 1/6886
USPC 514/10.4; 435/6.14; 435/325; 435/6.11

(57) ABSTRACT

Provided are methods and assays for cancer cell classification, cancer prognosis and treatment based on the isolation of circulating tumor cells and the characterization of their nuclear organization and telomere signatures.

### CROSS-REFERENCE TO RELATED APPLICATION

The present application claims the benefit of priority of U.S. Provisional Application No. 61/637,692 filed Apr. 24, 2012, and Canadian Patent Application No. 2,775,315 filed Apr. 24, 2012, which are each herein incorporated by reference.

### FIELD

The present application relates to assays, methods and systems for cancer cell classification, cancer prognosis and treatment based on the isolation of circulating tumor cells and the characterization of their nuclear organization and telomere signatures.

### INTRODUCTION

### Prostate Cancer

Prostate cancer is the second leading cause of death in men. However, there has been little progress in improving death rates from prostate cancer in the last fifty years.

During this time, through active screening programs (PSA and physical examination) there have been large numbers of men diagnosed with indolent prostate cancer which has been treated aggressively, with significant morbidity/mortality, because of the lack of a biomarker of aggressiveness. Prostate cancer is not health threatening in the majority of men.

Currently, no single marker/combination of biomarkers is able to predict disease behavior. Prostate-specific antigen (PSA) is too nonspecific (Berthold et al., 2008; Scher et al., 2009; Goodman et al., 2009). Other commonly used markers include the assessment of gene rearrangements involving TMPRSS22-ERG or ETS, PTEN loss, AR amplification, and increased chromosomal instability (for review, see Danila et al., 2011). However, none of these markers provides the complete picture of a patient's prostate cancer due to its heterogeneity and due to the presence or absence of these markers at certain stages during the course of the disease.

### Circulating Tumour Cells

Circulating tumor cells (CTCs) are cells that have detached from a primary tumor and circulate in the bloodstream. CTCs are rare cells. For example, one CTC may be present in one billion normal blood cells (Danila et al., 2011). CTCs may constitute seeds for subsequent growth of additional tumors (metastasis) in different tissues.

Different approaches have been taken to isolate CTCs or to demonstrate their presence indirectly. One commonly cited assay uses an anti-EpCAM antibody to magnetically capture CTCs expressing this antigen on their surfaces with the CellSearchR system (Scher et al., 2005; Berthold et al., 2008; Madan et al., 2011; Fleming et al., 2006; Gulley and Drake, 2011; Bubley et al., 1999; Scher et al., 2008). The draw-backs of this method lie in tumor cell heterogeneity, low EpCAM expression levels on CTCs, EpCAM expression level changes as cells become CTCs, and the possible selection of cells that express the “right” amount of EpCAM since only those will be captured by this method.

Other approaches rely on the presence of circulating nucleic acids (Schwarzenbach et al., 2011), on immunohistochemistry with anti-cytokeratin 8 and 18 antibodies that are also used in combination with the anti-EpCAM antibodies, or on CTC-chips. Another technology, the EPISPOT test, depletes CD45 cells first and examines the remaining cells. In addition, collagen adhesion matrix assays (CAM assays) have been introduced (for a review on these methods, see Doyen et al., 2011).

Recently, a new approach that isolates CTCs by size using a filter device that collects CTCs which can then be analyzed by cytomorphology, cell culture or molecular analyses has been developed (Desitter et al., 2011). This platform offers the possibility of examining all types of CTCs in a patient's blood sample and does not select a priori for sub-types.

### The Three-Dimensional (3D) Nuclear Organization of Telomeres

Telomeres are the ends of chromosomes. Functional telomeres prevent chromosomal fusions due to the presence of a protein complex, termed shelterin (de Lange, 2005). If any of the shelterin proteins are down-regulated or absent from the telomere, the complex is no longer protective, and affected telomeres become ‘reactive’ with other telomeres, and thus gain the ability to perform illegitimate fusion and/or recombination. Such telomeres become ‘dysfunctional’.

Telomere dysfunction is typical of cancer cells. When speaking of telomere dysfunction, one refers to critically shortened telomeres and/or to telomeres that lost their protective protein cap irrespective of their actual length (“uncapped” telomeres). When telomeres become dysfunctional, cells can become senescent, enter crisis or begin breakage-bridge-fusion cycles that initiate ongoing genomic instability (Misri et al., 2008; Deng et al, 2008: Lansdorp, 2009). Many cancer cells display chromosomal aberrations that are the direct result of telomere dysfunction. Examples include osteosarcoma (Selvarajah et al., 2006), prostate cancer (Vukovic et al., 2007; Vukovic et al., 2003), breast cancer (Meeker et al., 2004), and colon cancer (Stewenius et al., 2005; for reviews see, DePinho and Polyak, 2004; Lansdorp, 2009; Murnane and Sabatier, 2004).

Each nucleus has a telomeric signature that defines it as normal or aberrant (Mai and Garini, 2006; Mai and Garini, 2005; Louis et al., 2005). Four criteria define this difference; 1) nuclear telomere distribution, 2) the presence/absence of telomere aggregate(s), 3) telomere numbers per cell, and 4) telomere sizes (Mai, 2010).

To quantify the 3D nuclear organization of telomeres and to measure the above criteria defining the 3D nuclear organization, a semi-automated program, TeloView™ has been developed (Vermolen et al., 2005; Gonzalez-Suarez et al., 2009). Methods and systems for determining the 3D organization of telomeres are described in U.S. Pat. No. 7,801,682, issued Sep. 21, 2010 titled Method of Monitoring Genomic Instability Using 3D Microscopy and Analysis, which is incorporated herein by reference which is hereby incorporated entirety by reference. An automated version of TeloView™ designated TeloScan™ has also been developed which allows for high throughput analysis (Gadji et al., 2010; Klewes et al., 2011).

The ability to analyze the 3D nuclear organization of CTC cells is highly desirable. However, the question remains whether the physical handling of CTCs required in methods for the isolation of these rare cells leaves the nuclear structure of the CTC cells intact such that the three-dimensional nuclear organization of the telomeres of the CTC cells can be analysed. Indeed, sampling handling (for example, freezing) is known to alter the nuclear organization of cells.

A need remains for a robust and sensitive method for determining the 3D nuclear organization of CTC cells to obtain a telomeric signature of CTC subpopulations that can be used for example to correlate with clinical disease progression.

### SUMMARY OF THE DISCLOSURE

The present disclosure relates to the characterization of isolated circulating tumor cells (CTCs) in cancers including for example prostate cancer, breast cancer, melanoma, colon cancer, and lung cancer by isolating CTCs from the blood of a subject and determining the 3D telomere organization signature of the CTCs. It is demonstrated herein that one or more subpopulations of CTCs can be identified based on telomere profiles.

It is demonstrated herein the 3D quantitative fluorescence in situ hybridization analysis of CTCs isolated using a filtration device and the subsequent quantitative analysis of 3D telomeric profiles of CTCs leading to the identification of subgroups of CTCs is feasible. In addition the presence and frequency of circulating tumour microemboli in circulation can be estimated using the combination of CTC isolated by filtration and 3D analysis of CTCs.

Accordingly, disclosed herein are methods, systems and assays for cancer cell classification, cancer prognosis and treatment based on the nuclear organization and signatures of telomeres in CTCs. Also disclosed are methods for identifying sub-populations of CTCs based on their 3D telomere organization signature and isolated sub-populations obtained by the methods described herein.

The methods, assays and isolated sub-populations may for example allow for; 1) for the distinction of normal and tumor cells (Klewes et al., 2011), 2) for the identification of patient subgroups (Gadji et al., 2010) that will allow for new treatment design, 3) for the identification of patients who will recur and therefore should obtain different treatments (Knecht et al., 2010), 4) for treatment monitoring, and 5) for personalized medical management of patients (not one treatment for all, but a treatment specifically adapted to each patient).

The methods have been tested on CTCs of a number of cancers including prostate cancer, lung cancer, breast cancer, colon cancer and melanoma.

An aspect provides a method of identifying one or more circulating tumour cell (CTC) subpopulations comprising:
a. isolating CTCs from a blood sample from a subject;
b. determining the 3D telomere organization signature of a sample comprising measuring for each of a plurality of the isolated CTCs, one or more 3D telomere organization signature features selected from telomere number, telomere size, presence and/or number of telomeric aggregates, telomeres per nuclear volume, distances from nuclear centre, and a/c ratio; and
c. identifying one or more sub-populations of the CTCs based on the 3D telomere organization signature features of the CTCs.

In an embodiment, determining the 3D telomere organization signature of the sample further comprises calculating sample feature values optionally percentage of cells with telomeric aggregates, average number of telomeric aggregates per cell, average number of telomeres per cell and/or average nuclear volume.

In an embodiment the method further comprises isolating one or more sub-population(s) identified in step (c).

In another embodiment, the CTCs are isolated from the blood sample using a filter device.

In yet another embodiment, the CTCs are from a subject with prostate cancer, melanoma, breast cancer, colon cancer or lung cancer.

In an embodiment the sub-population of CTCs is identified based on the telomere organization signature feature telomere intensity. In another embodiment, the sub-population of CTCs is identified based on at least one of telomere number, telomere size and the presence and/or number of telomere aggregates. In another embodiment, the sub-population of CTCs is identified based on the 3D telomere organization signature feature telomere size.

In an embodiment, the method of identifying one or more CTC subpopulations comprises identifying:
i. a first sub-population comprising CTCs with an average telomere intensity of less than about 40,000, less than about 30,000, less than about 20,000, less than about 15,000, less than about 10,000 or less than about 5,000 a.u.;
ii. a second sub-population comprising CTCs with an average telomere intensity of from about 5,000-40,000 to about 30,000-60,000 or greater a.u.; and optionally
iii. a third sub-population comprising CTCs with an average telomere intensity of more than about 20,000, more than about 25,000, more than about 30,000, more than about 40,000, more than about 50,000 or more than about 60,000 a.u.

In another embodiment, the method of identifying one or more CTC subpopulations comprises identifying:
i. a first sub-population comprising CTCs with an average telomere intensity of less than about 20,000, less than about 25,000, less than about 30,000, less than about 35,000 or less than about 40,000 a.u.; and optionally
ii. a second sub-population comprising CTCs with an average telomere intensity of more than about 20,000, more than about 25,000, more than about 30,000, more than about 35,000 or more than about 40,000 a.u.

In another embodiment the method of identifying one or more CTC subpopulations, wherein the tumor cell is a prostate cancer cell, comprises identifying:
i. a first sub-population comprising CTCs with an average telomere intensity of less than about 20,000 a.u. or less than about 10,000;
ii. a second sub-population comprising CTCs with an average telomere intensity of about 10,000-20,000 a.u. to about 20,000-50,000 or greater a.u.; and optionally
iii. a third sub-population comprising CTCs with an average telomere intensity of more than about 20,000 or 50,000 a.u.

In another embodiment the method of identifying one or more CTC subpopulations, wherein the tumor cell is a colon cancer cell, comprises identifying:
i. a first sub-population comprising CTCs with an average telomere intensity of less than about 10,000 a.u.;
ii. a second sub-population comprising CTCs with an average telomere intensity of about 10,000 to about 35,000 or greater a.u.; and optionally
iii. a third sub-population comprising CTCs with an average telomere intensity of more than about 35,000 a.u.;

In another embodiment the method of identifying one or more CTC subpopulations, wherein the tumor cell is a breast cancer cell, comprises identifying:
i. a first sub-population comprising CTCs with an average telomere intensity of less than about 20,000 a.u. or less than about 10,000 a.u.;
ii. a second sub-population comprising CTCs with an average telomere intensity of about 10,000-20,000 a.u. to about 40,000-50,000 or greater a.u.; and optionally
iii. a third sub-population comprising CTCs with an average telomere intensity of more than about 40,000 a.u. or more than about 50,000 a.u.

In another embodiment the method of identifying one or more CTC subpopulations, wherein the tumor cell is a melanoma cancer cell, comprises identifying:
i. a first sub-population comprising CTCs with an average telomere intensity of less than about 20,000 or less than about 40,000 a.u.;
ii. a second sub-population comprising CTCs with an average telomere intensity of about 20,000-40,000 a.u. to about 40,000-60,000 or greater a.u.; and optionally
iii. a third sub-population comprising CTCs with an average telomere intensity of more than about 40,000 a.u. or more than about 60,000 a.u;.

In another embodiment the method of identifying one or more CTC subpopulations, wherein the tumor cell is a lung cancer cell, comprises identifying:
i. a first sub-population comprising CTCs with an average telomere intensity of less than about 10,000 a.u.;
ii. a second sub-population comprising CTCs with an average telomere intensity of about 10,000 to about 30,000 or greater a.u.; and optionally
iii. a third sub-population comprising CTCs with an average telomere intensity of more than about 30,000 a.u.

Another aspect provides a method for identifying the number of CTC subpopulations in a sample, the method comprising:
a. determining a 3D telomere organization signature of the sample comprising:
   a. obtaining a plurality of 3D telomere organization signature image datasets, each image dataset corresponding to a unique isolated CTC;
   b. determining values for 3D telomere organization signature features from the 3D telomere organization signature image datasets; and
b. determining a number of subpopulations in the sample based on one or more of the values of the features.

In another embodiment the 3D telomere organization signature features comprise at least one of telomere number, telomere size, presence and/or number of telomeric aggregates, telomeres per nuclear volume, distances from nuclear centre, and/or a/c ratio and/or the step of determining the 3D telomere organization signature further comprises calculating one or more sample feature values selected from percentage of cells with telomeric aggregates, average number of telomeric aggregates per cell, average number of telomeres per cell average nuclear volume.

In another embodiment, the features comprise at least one of telomere numbers, telomere intensities and telomeric aggregate numbers.

In an embodiment, the plurality of 3D telomere organization signature image datasets comprises at least 30 datasets.

In another embodiment, the subpopulations are assigned comparing one or more of telomere numbers, sizes, nuclear volumes, telomere distribution within the nucleus and/or nuclear sizes.

Another aspect provides an isolated sub-population of circulating tumour cells (CTCs) obtained by:
a. isolating a population of CTCs from the blood of a subject;
b. determining the 3D telomeres organization signature of the population of CTCs; and
c. isolating a sub-population of the CTCs based on one more of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In an embodiment, the sub-population comprises CTCs with an average telomere intensity of less than about 40,000, less than about 20,000, less than about 15,000, less than about 10,000 or less than about 5,000 a.u.

In another embodiment, the sub-population comprises CTCs with an average telomere intensity of about 5,000-40,000 to about 30,000-60,000 a.u.

In yet another embodiment, the sub-population comprises CTCs with an average telomere intensity of more than about 20,000, more than about 25,000, more than about 30,000, more than about 40,000, more than about 50,000 or more than about 60,000 a.u.

In an embodiment, the sub-population comprises CTCs with an average telomere intensity of more than about 20,000, more than about 25,000, more than about 30,000, more than about 35,000 or more than about 40,000 a.u. or less than about 20,000, less than about 25,000, less than about 30,000, less than about 35,000 or less than about 40,000 a.u.

Another aspect provides an assay comprising:
a. determining a 3D telomeres organization signature for a plurality of isolated test CTCs isolated from a blood sample from a subject with cancer;
b. identifying one or more subpopulations according to a method described herein; and
c. comparing the 3D telomeres organization signature of the test CTC subpopulations with a reference 3D telomeres organization signature, and if there is a difference or similarity in the 3D telomeres organization signature of the test CTCs and the reference 3D telomeres organization signature, identifying the subject as having an increased probability of a positive or negative clinical outcome.

In an embodiment, the clinical outcome is progression. In another embodiment the clinical outcome is recurrence.

In an embodiment, the 3D telomeres organization signature comprises values for one or more of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In another embodiment, the 3D telomeres organization signature comprises values for one or more of telomere numbers, telomere size and number of aggregates, and wherein an aberrant number of telomere, a decrease in average telomere size and/or an increased number of aggregates in the 3D telomeres organization signature of the test CTCs is indicative of an increased probability of a negative clinical outcome.

In an embodiment, the presence of telomere aggregates in at least 35%, 40%, 45%, 50%, 55%, 60%, 70% or 80% of the test CTCs is indicative of an increased probability of a negative clinical outcome.

In another embodiment, the assay further comprises identifying the number of CTCs in the blood sample and wherein more than about 25, more than about 30, more than about 35, more than about 40, more than about 45, more than about 50, more than about 60, more than about 70 or more than about 80 CTCs in 3.5 mL of blood is indicative of an increased probability of a negative clinical outcome.

In an embodiment, the population of test CTCs is organized into sub-populations based on telomere size and more than 2, 3, 4 or 5 sub-populations is indicative of an increased probability of a negative clinical outcome.

Another aspect provides a method of prognosing a clinical outcome in a subject with cancer comprising:
a. isolating CTCs from a sample from the subject to obtaining test sample CTCs, and
b. determining a 3D telomere organization signature of the test sample CTCs using 3D q-FISH;
c. identifying a clinical outcome of the subject according to the 3D telomere organization signature of the test sample CTCs.

In an embodiment, the clinical outcome is progression. In another embodiment the clinical outcome is recurrence.

In an embodiment, the CTCs are isolated from the blood sample using a filter device.

In another embodiment, the method further comprises step c), comparing the 3D telomere organization signature of the test sample CTCs with a 3D telomere organization signature in a control, wherein a difference or similarity in the 3D telomere organization signature between the test sample CTCs and the control is indicative of the clinical outcome of the subject.

In an embodiment, the cancer is melanoma, colorectal cancer, lung cancer, breast cancer or prostate cancer.

In another embodiment, the 3D telomeres organization signature comprises one or more of telomere numbers, telomere size and number of aggregates, and an aberrant number of telomere, a decrease in average telomere size and/or an increased number of aggregates in the 3D telomeres organization signature of the test CTCs is indicative of an increased probability of a negative clinical outcome.

In another embodiment, the presence of telomere aggregates in at least 35%, 40%, 45%, 50%, 55%, 60%, 70% or 80% of the test CTCs is indicative of an increased probability of a negative clinical outcome.

In an embodiment, the assay further comprises identifying the number of CTCs in the blood sample and wherein more than about 25, more than about 30, more than about 35, more than about 40, more than about 45, more than about 50, more than about 60, more than about 70 or more than about 80 CTCs in 3.5 mL of blood is indicative of an increased probability of a negative clinical outcome.

In another embodiment, a population of test CTCs that comprises more than 2, 3, 4 or 5 sub-populations, wherein the sub-population is based on telomere size, is indicative of an increased probability of a negative clinical outcome.

In an embodiment, the method further comprises assessing the number of circulating tumour microemboli, wherein an increased number of circulating tumour microemboli is a poor prognosticator. For example, the presence of one or more circulating tumor microemboli is a poor prognosticator. The higher the number of microemboli the worse the potential outcome.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

In another aspect is provided a method of treating a subject, comprising prognosing the clinical outcome of a subject according to the method described herein and providing a suitable treatment according to the prognosis.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1.1 (a) and (b). 2D and 3D telomere FISH on H2030 non-small cell lung carcinoma CTCs isolated with the filter device of Desitter E. et al. (c) Telomere number versus intensity in H2030 non-small cell lung carcinoma CTCs. Three sub-populations of small, intermediate and large telomeres based on telomere intensities are marked.

FIG. 1.2. (a) 2D and 3D telomere FISH on LIM F2538 melanoma CTCs isolated with the filter device of Desitter E. et al. (b) Telomere number versus intensity in LIM F2538 melanoma CTCs. Three sub-populations of small, intermediate and large telomeres based on telomere intensities are marked.

FIG. 1.3. (a) 2D and 3D telomere FISH on RAV F3885 breast cancer CTCs isolated with the filter device of Desitter E. et al. (b) Telomere number vs. intensity in RAV F3885 breast cancer CTCs. Three sub-populations of small, intermediate and large telomeres based on telomere intensities are marked.

FIG. 1.4. 3D telomere FISH and chart of telomere number vs. intensity in MIC 10AA3956 breast cancer CTCs.

FIG. 1.5. 3D telomere FISH and chart of telomere number vs. intensity in WUR 10AA2499 breast cancer CTCs.

FIG. 1.6. 3D telomere FISH and chart of telomere number vs. intensity in colon cancer CTCs.

FIG. 1.7. H&E-stained filters with isolated CTCs and CTC clusters pointed out. Panels A and B show filtered prostate and colon cancer CTCs surrounded by pores of the filters. The shapes and sizes of the CTCs can be acknowledged. Panels C and E show clustered breast CTCs and lung cancer cell line captured by the filter. Panel D shows a melanoma microemboli.

FIG. 2A. Shows 2D (Aa, Ac, and Ae) and 3D (Ab, Ad, and Af) images of sample MB0181PR prostate cancer CTCs. At least three different subpopulations of CTCs were identified and depicted in this patient. Images Aa and Bb show cells with scanty telomeres in contrast to Ac and Ad, which represent population of CTCs with higher telomere intensity. In images Ae and Af, the cells show the presence of TAs, thus signal intensities are even higher than seen in Ac and Ad.

FIG. 2B. Shows 2D and 3D representations of different subpopulations in colon cancer patient sample GUI3F. In the 2D images (Ba, Bc, and Be) the telomere signals are represented by the dots and the corresponding signals of all image stacks are shown in the 3D images beside them. There is a graduated increase in signal intensity along the CTC subpopulations shown in Bb, Bd, and Be, respectively. Image Bb represents the low extreme, whereas image Be shows an extremely high number of signals with a shorter than normal telomeres, which is characteristic of advanced cancer stage.

FIG. 2C. Shows representations of three different subpopulations of CTCs in the same breast cancer patient (Br 3934 MIC) in both 2D and 3D images. The signal intensity increases from Ca and Cb to Cc and Cd with increased numbers of telomeres. Subpopulation of cells with high number of telomere aggregates (TAs) is represented by image Cf.

FIG. 2D. Shows 2D and 3D images of melanoma CTCs showing different subpopulations. The nuclear architecture is outlined by DAPI in the 2D images. Images Da and Db have fewer telomeres; more signals are present in subpopulation represented by image Dd with few TA formation. The last subpopulation represented here by image Df has many TAs giving it a high signal intensity.

FIG. 2E. Shows representative images of lung cancer cell line, which were found to be generally larger than the other tumors examined. All the subpopulations found in the H2030 lung cancer examined had more than normal number of telomeres. The different subpopulations are identified using the difference in signal intensities in the nuclei with the low signals represented by images Ea and Eb, medium intensity by Ec and Ed, and high intensity subpopulation as images Ee and Ef.

FIG. 3A. Shows a plot of telomere numbers against telomere intensities for Prostate CA (MB0181PR). The uniqueness of the tumor types, as compared to the tumor types in FIG. 3B-3E, is depicted in the plots and the multiple peaks indicate the different subpopulations of telomeres present in the same patient blood sample. Note that the scales of the graphs in FIG. 3A-3E are not the same; they were adjusted to give a clear presentation of the subpopulations observed in each patient sample.

FIG. 3B. Shows a plot of telomere numbers against telomere intensities for Colon CA (GUI3F). The uniqueness of the tumor type, as compared to the tumor types in FIGS. 3A and 3C-3E, is depicted in the plot and the multiple peaks indicate the different subpopulations of telomeres present in the same patient blood sample. Note that the scales of the graphs in FIG. 3A-3E are not the same; they were adjusted to give a clear presentation of the subpopulations observed in each patient sample.

FIG. 3C. Shows a plot of telomere numbers against telomere intensities for Breast CA (MIC 10AA3934). The uniqueness of the tumor type, as compared to the tumor types in FIGS. 3A, 3B and 3D-3E, is depicted in the plot and the multiple peaks indicate the different subpopulations of telomeres present in the same patient blood sample. Note that the scales of the graphs in FIG. 3A-3E are not the same; they were adjusted to give a clear presentation of the subpopulations observed in each patient sample.

FIG. 3D. Shows a plot of telomere numbers against telomere intensities for Melanoma (CAR 10AA2213). The uniqueness of the tumor type, as compared to the tumor types in FIG. 3A-3C and 3E, is depicted in the plot and the multiple peaks indicate the different subpopulations of telomeres present in the same patient blood sample. Note that the scales of the graphs in FIG. 3A-3E are not the same; they were adjusted to give a clear presentation of the subpopulations observed in each patient sample.

FIG. 3E. Shows a plot of telomere numbers against telomere intensities for Lung CA (H2030). The uniqueness of the tumor type, as compared to the tumor types in FIG. 3A-3D, is depicted in the plot and the multiple peaks indicate the different subpopulations of telomeres present in the same patient blood sample. Note that the scales of the graphs in FIG. 3A-3E are not the same; they were adjusted to give a clear presentation of the subpopulations observed in each patient sample.

FIG. 4. Shows a comparison of 3D nuclear telomere profiles of CTCs isolated from prostate cancer patient MB0239PR with lymphocytes from the same patient captured on the same filter. Triangles indicate the 3D nuclear telomere profile of CTCs from the patient. Squares indicate the 3D nuclear profile of lymphocytes from the same patient. The average 3D volume of lymphocytes is 211.47 μm3 and the nuclear diameter is 7.39 μm. The ANV of CTCs of this patient is 665.99 μm3 and the average nuclear diameter is 10.78 μm. The ANV of lymphocytes is 3.15 times smaller than that of average CTC in this same patient. Note that due to filtration, 10% less telomeric signals are detectable in 3D nuclei in normal lymphocytes than have been reported by us and others (Chuang et al., 2004; de Vos et al., 2009; Vermolen et al., 2005).

FIG. 5. 3D nuclear telomere analysis of prostate cancer CTCs from sample MB 10A 1975 isolated using the methods of Desitter E. et al. The data highlight the presence of CTC sub-populations with small, small and intermediate and intermediate/large and large telomeres respectively. (a) to (c) 2D images of CTCs captured. (a′) to (c′) Telomeres of CTCs shown in a-c, visualized by 3D imaging. Solid arrows point to very short telomeres; dashed arrow points to a telomeric aggregate in c. (d) Overview graph of telomere numbers and intensities measured in isolated CTCs. Three sub-populations of small, intermediate and large telomeres based on telomere intensities are marked and correspond to (c), (a) and (b), respectively. (e) Normal nucleus and telomeres.

FIG. 6. Comparison of two cases of prostate cancer CTCs. MB 10A 1975 (also shown in FIG. 5) has metastatic high grade prostate cancer, and MB 10A 2004 has intermediate risk localized disease. The numbers of CTCs are higher in MB 10A 1975 (>40/3.5 ml of blood) than MB 10A 2004 (30/3.5 ml blood). There are three sub-populations in MB 10A 1975 based on telomere intensities (0-10000; 10001-20000; 20001 to 80000) and two in MB 10A 2004 (0-30000 and 30001-80000). The complexity of telomere dysfunction is greater in MB 10A 1975.37% of cells have aggregates in MB 10A 2004 while the number is 46% in MB 10A 1975.

FIG. 7 is a diagram of an example embodiment of an apparatus that can be used determine 3D telomere organization.

FIG. 8A is a flowchart of an example embodiment of a method that can be employed to identify CTC subpopulations.

FIG. 8B is a flowchart of an example embodiment of a method that can be used to determine 3D telomere organization.

FIG. 9 shows a plot of telomere numbers against telomere intensities for a Group I prostate cancer patient. In the left inset is a 2D image of the CTC showing the nuclear architecture in light grey stained with DAPI and telomeres as dots. In the right inset are the telomeres of the CTC visualized by 3D imaging.

FIG. 10 shows a comparison plot of telomere numbers against telomere intensities for a Group I prostate cancer patient and a normal lymphocyte cell. In the inset is a 2D microscopic image of the CTCs and lymphocytes showing their relative sizes.

FIG. 11 shows a plot of telomere numbers against telomere intensities for a Group II prostate cancer patient. In the insets are shown 3D visualizations of the telomeres from the two distinct subtypes of CTCs.

FIG. 12A are shown 3D visualizations of the telomeres from the three distinct subtypes of CTCs observed in Group III prostate cancer patients.

FIG. 12B shows a plot of telomere numbers against telomere intensities for a Group III prostate cancer patient.

FIG. 13-20 show two overlayed plots of telomere numbers against telomere intensities for prostate cancer patients from samples taken six months apart.

### DETAILED DESCRIPTION

### I. Definitions

As used herein, the term “cell” includes more than one cell or a plurality of cells or portions of cells. The sample may be from any animal, in particular from humans, and may be biological fluids (such as blood, serum, or bone marrow), tissue, or organ.

The term “circulating tumor cell” (CTC) as used herein refers to a cancer cell derived from a cancerous tumor that has detached from the tumor and is now circulating in the blood stream of a subject. A CTC may be derived from any type of cancer including but not limited to prostate cancer, lung cancer, breast cancer, colon cancer and melanoma.

The term “control” as used herein refers to a suitable comparator subject, sample, cell or cells such as non-cancerous subject (or earlier stage cancer subject, sample, cell or cells), or blood sample, cell or cells from such a subject, for comparison to a cancer subject, sample (e.g. test sample) cell or cells from a cancer subject; or an untreated subject, cell or cells, for comparison to a treated subject, cell or cells, according to the context. Control can also refer to a reference value or set of reference values (e.g. reference 3D telomeres organization signature values) derived from and representative of a control subject, cell and/or cells and/or a population of subjects with a known outcome, and/or a subject base-line value(s). In an embodiment, the reference value is a base-line value for a subject that is used for monitoring changes. The term “control cell” is a suitable comparator cell e.g. a cell that is known of not having a cancer such as prostate cancer (e.g. negative control), including for example a non-cancerous cell from the subject being tested such as a lymphocyte cell; or a cell or population of cells that is known as having a cancer such as prostate cancer or a precursor syndrome (e.g. positive control) that is used as comparison or for determining a threshold for a particular cancer or population. A positive “control cell” may be a tumor cell of a known stage and/or progression. Control tumor cells of known stage and/or progression may be used to generate thresholds for tumor stage and/or progression.

The term “cancer” as used herein means a metastatic and/or a non-metastatic cancer, and includes primary and secondary cancers. Reference to cancer includes reference to cancer cells.

The term “prostate cancer” as used herein refers to cancers that originate in the prostate gland and includes primary and secondary cancers. Reference to prostate cancer includes reference to prostate cancer cells.

The term “breast cancer” as used herein refers to cancers that originate in the tissues of the breast and includes primary and secondary cancers. Breast cancer is a cancer that starts in the tissues of the breast. Examples of breast cancers include ductal carcinoma and lobular carcinoma. Reference to breast cancer includes reference to breast cancer cells.

The term “lung cancer” as used herein refers to cancers that originate in the lung and includes primary and secondary cancers. Reference to lung cancer includes reference to lung cancer cells.

The term “colon cancer” or “colorectal cancer” as used herein refers to cancer that originates in the large intestine (colon) or the rectum (end of the colon) and includes primary and secondary cancers. Reference to colon cancer or colorectal cancer includes reference to colon cancer or colorectal cancer cells.

The term “melanoma” as used herein refers to malignant tumors of melanocytes and includes primary and secondary cancers. Melanocytes are cells that produce the dark pigment, melanin, which is responsible for the color of skin. Melanoma can originate in any part of the body that contains melanocytes. Reference to melanoma includes reference to melanoma cells.

The term “prognosis” as used herein refers to an expected course of clinical disease. The prognosis provides an indication of disease progression and includes for example, an indication of likelihood of recurrence, metastasis, death due to disease, tumor subtype or tumor type. The prognosis can comprise a good prognosis which corresponds to a good clinical outcome relative to the spectrum of possible clinical outcomes for the specific, and a poor prognosis, which corresponds to a poor clinical outcome relative to the spectrum of possible clinical outcomes for the specific cancer. As used herein, “good prognosis” means a probable course of disease or disease outcome that has reduced morbidity and/or reduced mortality compared to the average for the disease or condition. As used herein, “poor prognosis” means a probable course of disease or disease outcome that has increased morbidity and/or increased mortality compared to the average for the disease or condition.

The term “aggressive cancer” as used herein refers to a cancer with a poor prognosis. An aggressive cancer can include a cancer which progresses quickly, has a high likelihood of reoccurrence, metastasis and death due to disease and is refractory to treatment.

The term “non-aggressive cancer” as used herein refers to a cancer with a good prognosis. A non-aggressive cancer can include a cancer which progresses slowly, has a low likelihood of recurrence, metastasis and death due to disease and is responsive to treatment.

The term “telomeric organization” as used herein refers to the 3D arrangement of the telomeres during any phase of a cell cycle and includes such parameters as alignment (e.g. nuclear telomere distribution), state of aggregation, telomere numbers per cell and/or telomere sizes, a/c ratios and/or nuclear volumes. “Telomere organization” also refers to the size and shape of the telomeric disk, captured for example in an a/c ratio and which is the organized structure formed when the telomeres condense and align during the late G2 phase of the cell cycle. The term “state of aggregation” refers to the presence or absence of telomere aggregate(s) and/or the size and shape of the aggregates of telomeres. The term “telomere aggregates” means telomeres found in clusters that at an optical resolution limit of 200 nm cannot be further resolved (Vermolen et al., 2005; Mai and Garini, 2006; Mai, 2010). As another example, telomere aggregates are defined as clusters of telomeres that are found in close association. Telomeric aggregates are not typically seen in normal cells.

The “difference in telomeric organization” between for example the sample and the control and/or in the test cell compared to the control cell and/or between cell subpopulations can be determined, for example by counting the number of telomeres in the cell, measuring the size or volume of any telomere or telomere aggregate, or measuring the alignment of the telomeres, and comparing the difference between the cells in the sample and the cells in the control. The differences in telomeric organization between the sample and the control can be measured and compared using individual cells or average values from a population of cells. For example, if any telomere in the test cell is larger (i.e. forms more aggregates), for example double the size, of those in the control cell, then this indicates the presence of genomic instability in the test cell. The telomeres in a test cell may also be fragmented and therefore appear smaller than those in the control cell. Accordingly, a change or difference in telomeric organization in the test cell compared to the control cell and/or between subpopulations can be determined by comparing parameters used to characterize the organization of telomeres. Such parameters are determined or obtained for example, using a system and/or method described herein below.

The term “telomere organization signature” as used herein refers to a 3D telomere organization which can be measured for example using TeloView™ or TeloScan™. It includes for example, values for one or more of the following features; telomere numbers, telomere intensities (sizes), overall telomere distribution, telomere aggregates, nuclear volumes. For example the features that define differences in telomere signatures include 1) nuclear telomere distribution, 2) the presence/absence of telomere aggregate(s) (telomere aggregates are telomeres found in clusters that at an optical resolution limit of 200 nm cannot be further resolved and which are not seen in normal cells), 3) telomere numbers per cell, and 4) telomere sizes. Additional criteria include a/c ratios (a/c ratios define the nuclear positions of telomeres). The a/c ratios are characteristic for specific cell cycle phases and nuclear volumes. Feature values for a sample can be calculated and include for example percentage of cells with telomeric aggregates, average number of telomeric aggregates per cell, average number of telomeres per cell, and/or average nuclear volume. Sample values allow for example comparison to other samples.

The term “a/c ratio” refers to a parameter that defines the nuclear position of a telomere. The a/c ratio is characteristic for a specific cell cycle phase (Vermolen et al., 2005).

The term “aggressive cancer telomere organization signature” as used herein refers to a telomere organization signature for cancer cells such as CTCs associated with an aggressive form of cancer. The term “non-aggressive cancer telomere organization signature” for cancer cells such as CTCs associated with a non-aggressive form of cancer.

An aggressive cancer telomere organization signature is characterized for example by a telomere number at 630× magnification in CTC cells of greater than about 10, greater than about 25, greater than about 30, greater than about 35, greater than about 40, greater than about 45, or greater than 50. The aggressive cancer telomere organization signature is characterized for example by decreased mean telomere intensity in CTC cells originating from an aggressive cancer compared to CTCs originating from a non-aggressive cancer. The aggressive cancer telomere organization signature is also characterized for example by an increased percentage of very short telomeres in CTC cells originating from an aggressive cancer compared to CTCs originating from a non-aggressive cancer. For example, an aggressive cancer telomere organization signature is characterized by greater than 60%, greater than 65%, greater than 70%, greater than 75%, or greater than 80% very short telomeres in CTC cells. For example, telomeres with a relative fluorescent intensity x-axis) ranging from 0-5,000 units can be classified as very short, with an intensity ranging from 5,000-15,000 units can be classified as short, with an intensity from 15,000-30,000 units can be classified as mid-sized, and with an intensity >30,000 units as large (18). The units are arbitrary units (e.g. a.u). As demonstrated herein, for example in Table 2 and the FIGs, sub-populations can comprise differing size classifications. The telomere aggregates at 630× magnification is also increased compared to the non-aggressive cancer telomeres organization signature, for example greater than 2.5, greater than 3, greater than 3.5, greater than 4, greater than 4.5, greater than 5, greater than 5.5 or greater than 6 in CTC cells (e.g. per cell) and greater than 2.5, greater than 3, greater than 3.5 or greater than 4 in CTC cells per unit volume. A non-aggressive cancer telomere organization signature is characterized for example by a telomere number at 630× magnification in CTCs of less than about 30, less than about 25, less than about 20, less than about 15, or less than about 10. The non-aggressive cancer telomere organization signature is characterized for example by increased mean telomere intensity in CTCs originating from a non-aggressive form of cancer, compared to CTCs originating from a more aggressive form of cancer. The non-aggressive cancer telomere organization signature is also characterized for example by a decreased percentage of very short telomeres in CTC cells compared to the aggressive cancer telomeres organization signature. For example, the non-aggressive cancer telomere organization signature is characterized by having less than about 70%, less than about 65%, less than about 60%, less than about 50% very short telomeres in CTC cells. The telomere aggregates (630× magnification) is also less, for example less than 4, less than 3.5, or less than 3, less than 2.5, less than 2, less than 1.5, less than 1, less than 0.5 in CTC cells per unit volume (or per cell).

The term “sub-population” as used herein refers to a subset of CTCs isolated from a sample, wherein the sub-population of cells includes cells that are similar with respect to at least one of the following properties: telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio. Optionally, a sub-population of CTC cells includes cells that have similar telomere organization signatures. The term “similar” optionally refers to measurements (for example, number of telomeres, telomere size etc) that fall within a specified range. Optionally, the term “similar” refers to measurements that fall within 5, 10, 15, 20, 30, 40, 50, 60, 70, 80 or 100% of the mean measurement or measurements that fall within 1, 2 or 3 standard deviations of the mean.

An example of a sub-population of CTCs is a sub-population of CTCs with an average telomere intensity of less than about 40,000, less than about 35,000, less than about 30,000, less than about 25,000, less than about 20,000, less than about 15,000, less than about 10,000 or less than about 5,000 a.u. In a further example, a sub-population of CTCs is a sub-population of CTCs with an average telomere intensity of more than about 40,000, more than about 35,000, more than about 30,000, more than about 25,000, more than about 20,000, more than about 15,000, more than about 10,000 or more than about 5,000 a.u. Another example of a sub-population of CTCs is a sub-population of CTCs with an average telomere intensity ranging from 5,000-40,000 to 30,000-60,000 a.u., for example ranging any number between 5,000 and 40,000 to any number between 30,000 and 60,000 a.u. (with the requirement that the end range number be larger than the start range number). Other examples of ranges are provided in the Examples and FIGs.

The term “sample” as used herein refers to any biological fluid comprising a cell, a cell or tissue sample from a subject that can comprise CTS. including a sample from a test subject, i.e. a test sample, such as from a subject with a cancer, or a control sample from a control subject, e.g., a subject without a cancer. The sample can comprise a blood sample, for example a peripheral blood sample, a fractionated blood sample, or a bone marrow sample. The sample volume is sufficient to comprise for example at least 20 cells, at least 25 cells or at least 30 cells or any number between 20 and 30.

The term “isolating CTCs” as used herein refers to the isolation of CTC cells from a sample such as a blood sample. Optionally, CTCs are isolated by size using a filter device. For example, in a filter device, blood flows passed a microporous membrane filter allowing size-selective isolation of CTCs. The isolated CTCs can then be analyzed by cytomorphology, cell culture or molecular analysis. One example of a filter device is ScreenCell's filter device as described in Desitter et al (2011). For example, since prostate cancer cells range in size from 15 to 25 microns they are captured on ScreenCell filters (Desitter et al., 2011; Zheng et al., 2007) allowing, for the first time, the ability to perform a detailed analysis of all CTCs present in blood samples (e.g. of the blood volume captured).

The term “subject” as used herein includes all members of the animal kingdom including mammals, and suitably refers to humans.

The term “three-dimensional (3D) analysis” as used herein refers to any technique that allows the 3D visualization of cells, for example involving high resolution deconvolution microscopy.

The term “mean telomere intensity” as used herein means a mean telomere relative fluorescent intensity (length) of all telomeres within a given volume.

The term “telomere length” or “telomere size” as used herein refers to the relative fluorescent intensity of telomeres. For example telomeres with a relative fluorescent intensity (x-axis) ranging from 0-5,000 units are classified as very short, with an intensity ranging from 5,000-15,000 units as short, with an intensity from 15,000-30,000 units as mid-sized, and with an intensity >30,000 units as large (Knecht H et al 2010). Other classifications can be used according for example to the cancer.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described are intended to be applicable to other embodiments herein described for which they are suitable as would be understood by a person skilled in the art. For example, in the above passages, different aspects of the disclosure are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods

It is demonstrated herein that the 3D nuclear organization of CTCs isolated from blood samples can be determined. The determination of the 3D nuclear organization of the isolated CTCs allows the grouping of the CTCs into sub-populations based for example on telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and/or a/c ratio. The 3D nuclear organization signatures and the resulting identification of sub-populations provide clinical information that can be used in the assessment of prognosing a clinical outcome in a subject with cancer.

Accordingly, disclosed herein is a method of identifying one or more circulating tumour cell (CTC) subpopulations comprising:
a. isolating CTCs from a blood sample from a subject;
b. determining the 3D telomere organization signature of each of a plurality of the isolated CTCs; and
c. identifying one or more sub-populations of the CTCs based on one or more of 3D telomere organization signature features selected from telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In an embodiment, the method of identifying one or more circulating tumour cell (CTC) subpopulations comprises:
a. isolating CTCs from a blood sample from a subject;
b. determining the 3D telomere organization signature of a sample comprising measuring for each of a plurality of the isolated CTCs, one or more 3D telomere organization signature features selected from telomere number, telomere size, presence and/or number of telomeric aggregates, telomeres per nuclear volume, distances from nuclear centre, and a/c ratio; and
c. identifying one or more sub-populations of the CTCs based on one or more of the 3D telomere organization signature features selected from telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In an embodiment, the CTCs are isolated from the blood sample using a filter and/or a marker based method.

For example, CTCs can be isolated using an anti-EpCAM antibody to magnetically capture CTCs expressing this antigen on their surfaces with for example the CellSearchR system (Scher et al., 2005; Berthold et al., 2008; Madan et al., 2011; Fleming et al., 2006; Gulley and Drake, 2011; Bubley et al., 1999; Scher et al., 2008) Other approaches include for example detecting the presence of circulating nucleic acids (Schwarzenbach et al., 2011), on immunohistochemistry with anti-cytokeratin 8 and 18 antibodies that are also used in combination with the anti-EpCAM antibodies, or on CTC-chips as well as the EPISPOT test, which depletes CD45 cells first and examines the remaining cells. In addition, collagen adhesion matrix assays (CAM assays) can be used (for a review on these methods, see Doyen et al., 2011).

In an embodiment, the CTCs are from a subject with prostate cancer, melanoma, breast cancer, brain tumour, colon cancer or lung cancer or any metastasing tumour. Ranges for different cancers can be determined as demonstrated here as it has been determined that CTCs isolated using for example filters, can be subjected 3D analysis using the methods described herein, including TeloView and/or TeloScan.

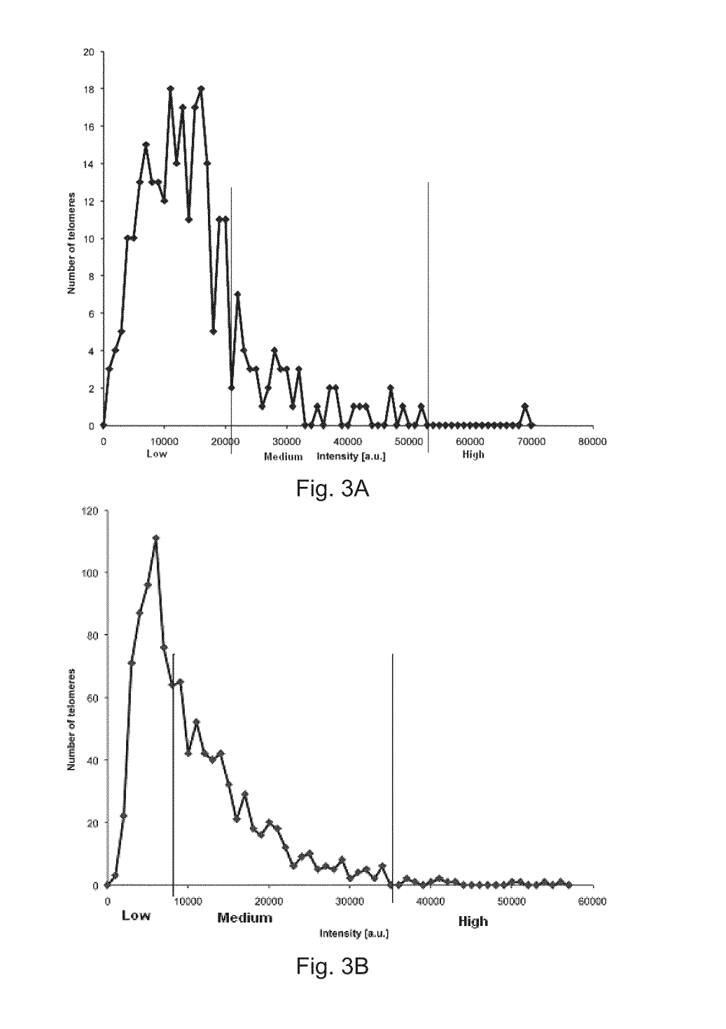

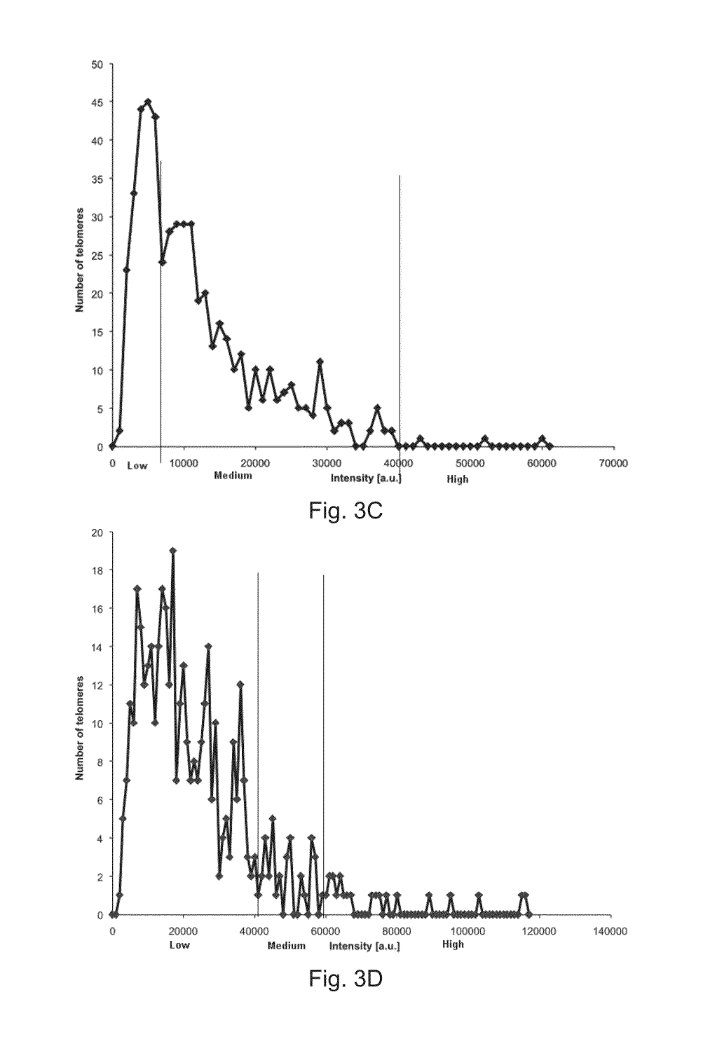

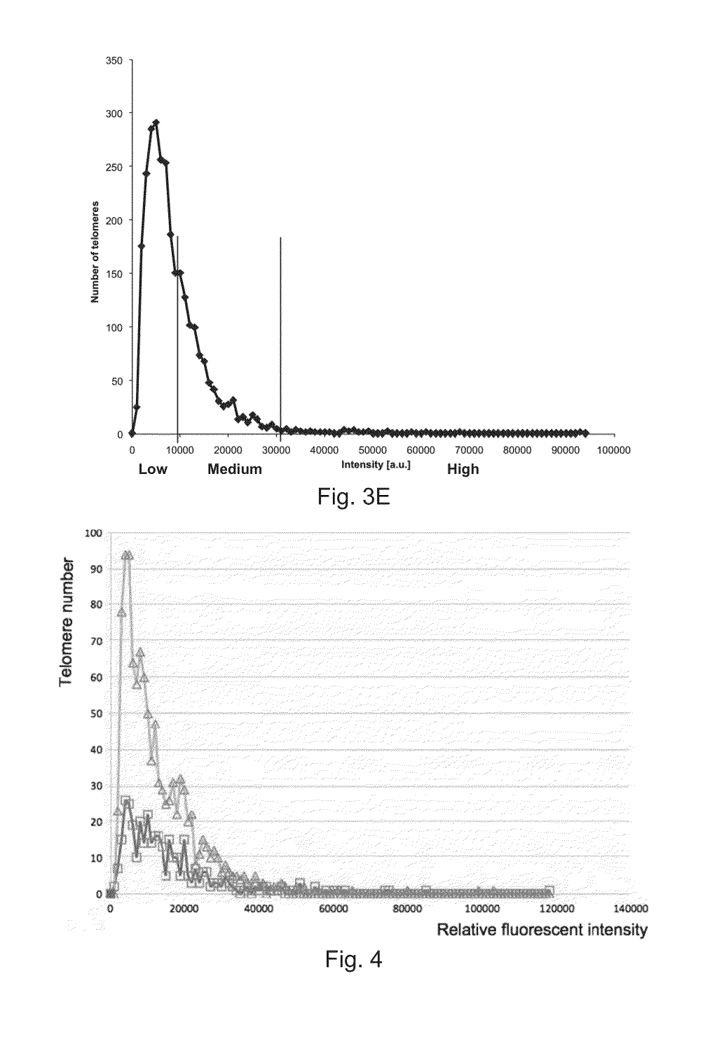

TeloView™ for example quantifies the telomere numbers, signal intensity, sizes, distribution, TAs, and nuclear volume for each sample. A graph of telomere numbers (y-axis) against signal intensity (x-axis) can be plotted for each sample giving a first overview of the CTC 3D telomere signature and of the presence/absence of subpopulations (see for example FIG. 3 A-E). In addition, aggregate numbers and nuclear volumes are calculated and included in the analysis.

Statistical parameters considered for characterizing the CTCs in each subject ample into subpopulations include for example: 1) percentage of cells with aggregates (PCA), 2) average number of telomeres per cell (ANTC), 3) average number of aggregates per cell (ANAC), and 4) average nuclear volume (ANV).

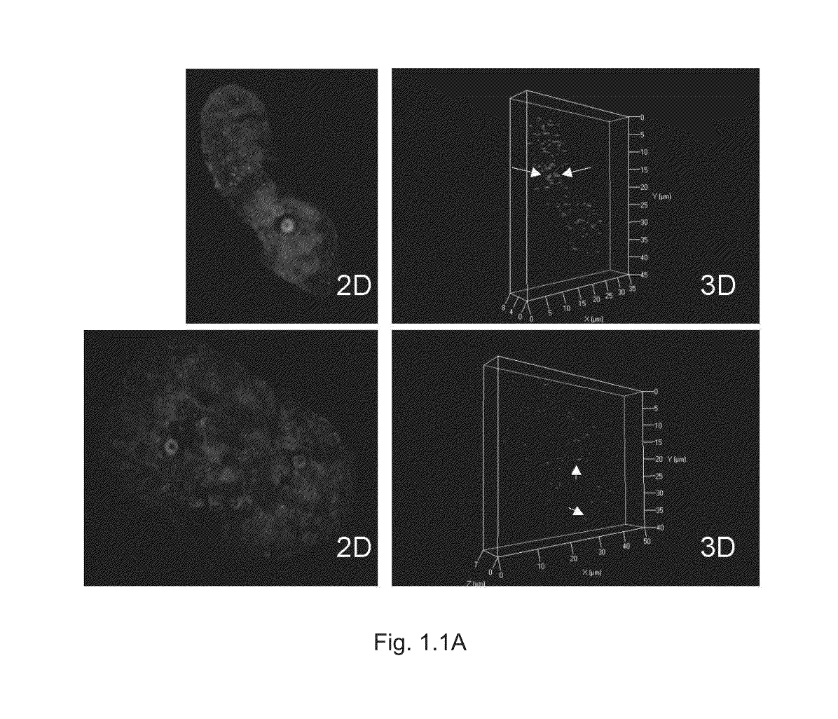

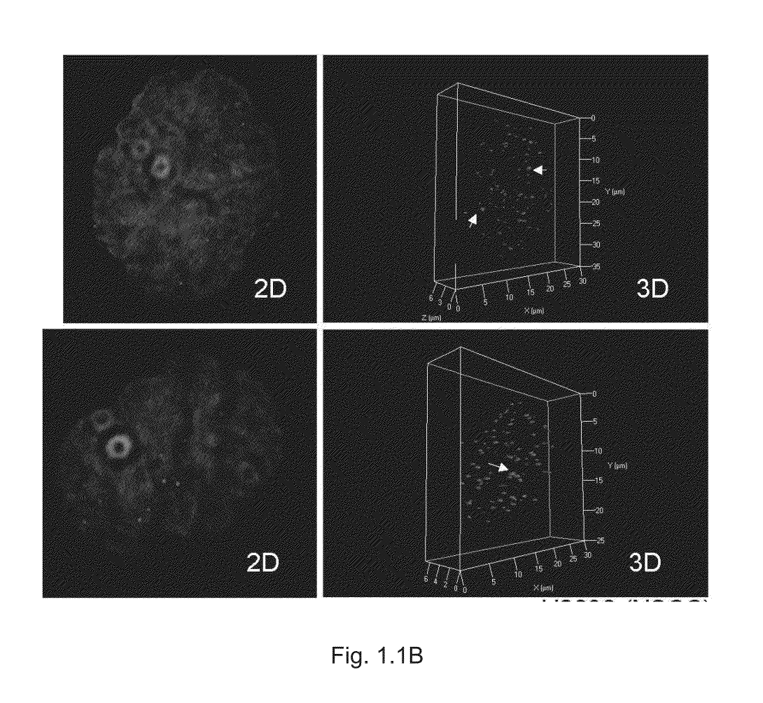

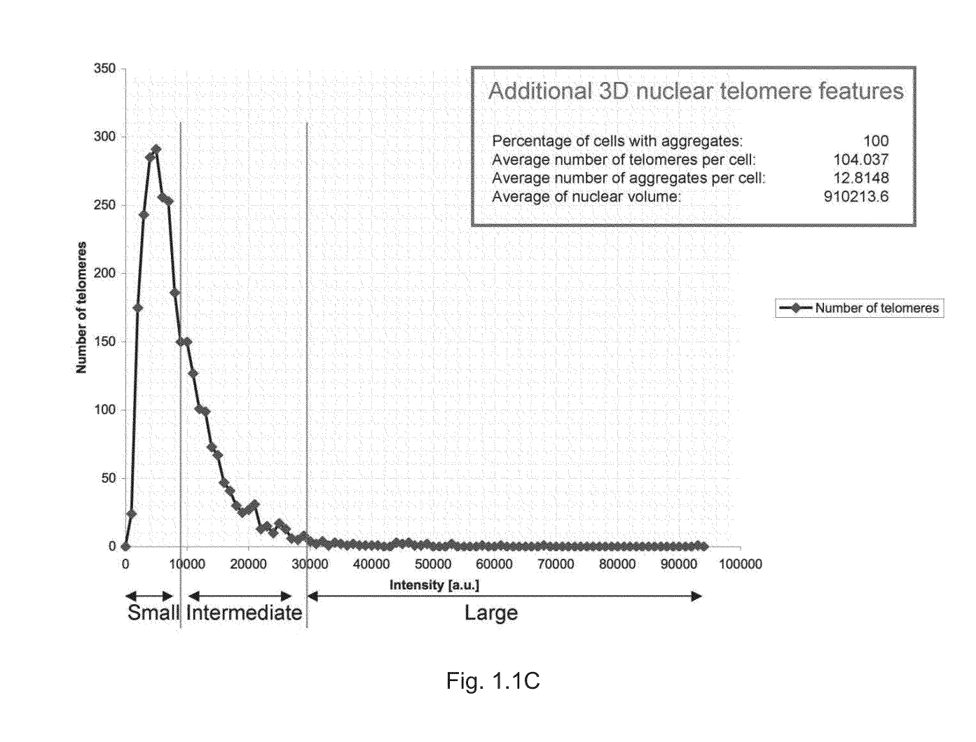

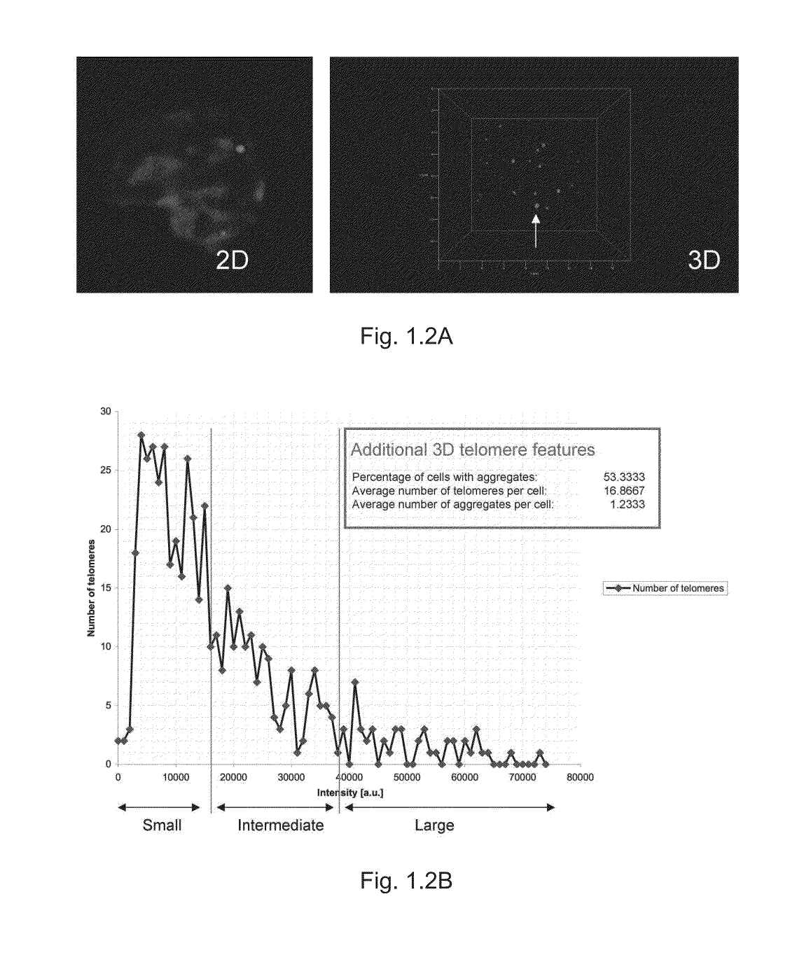

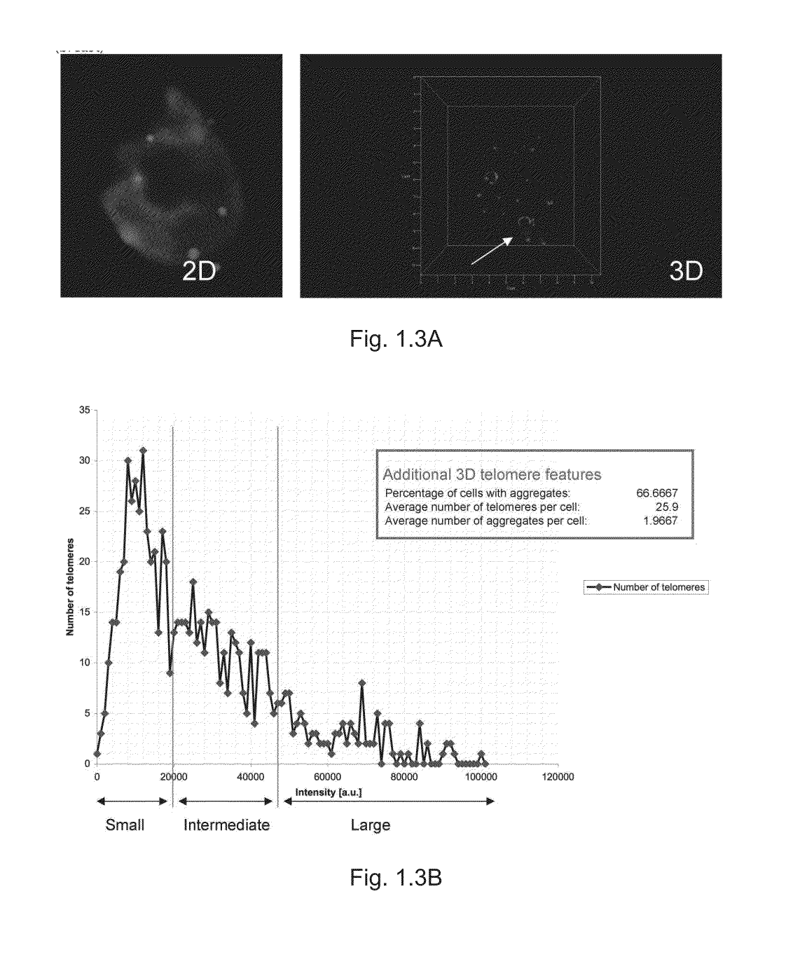

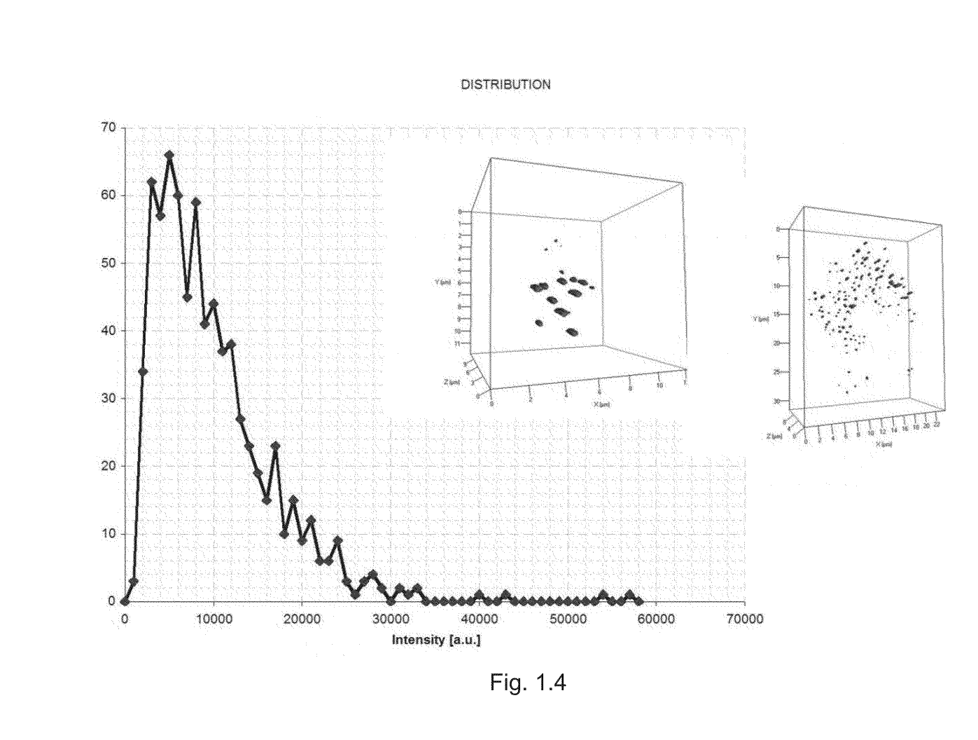

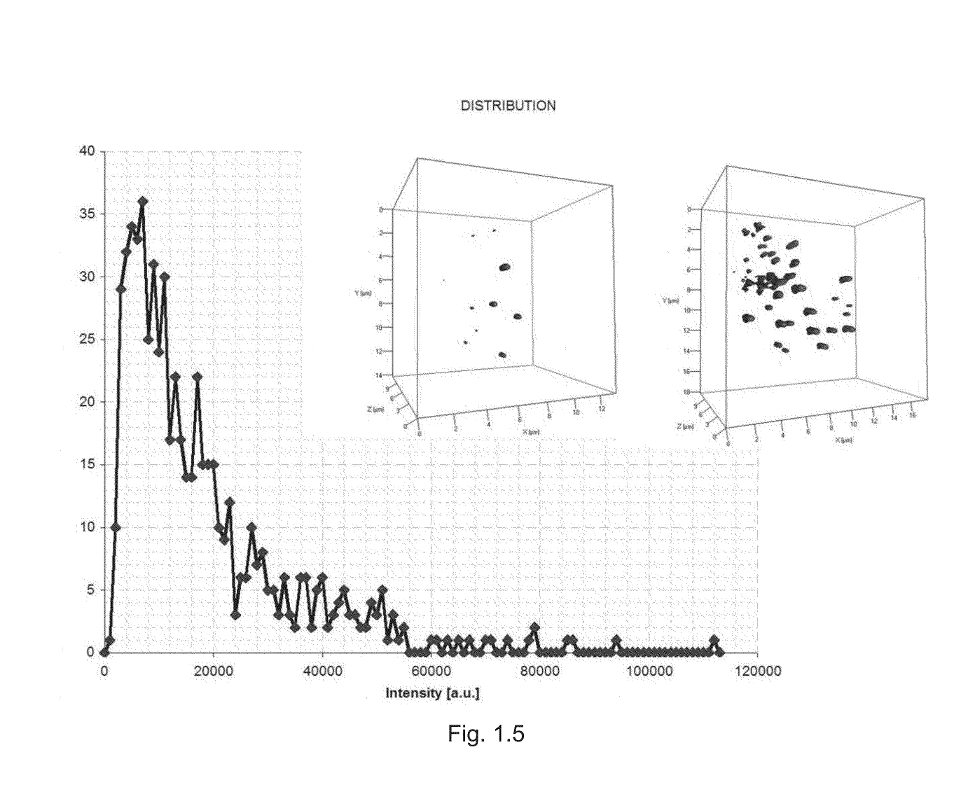

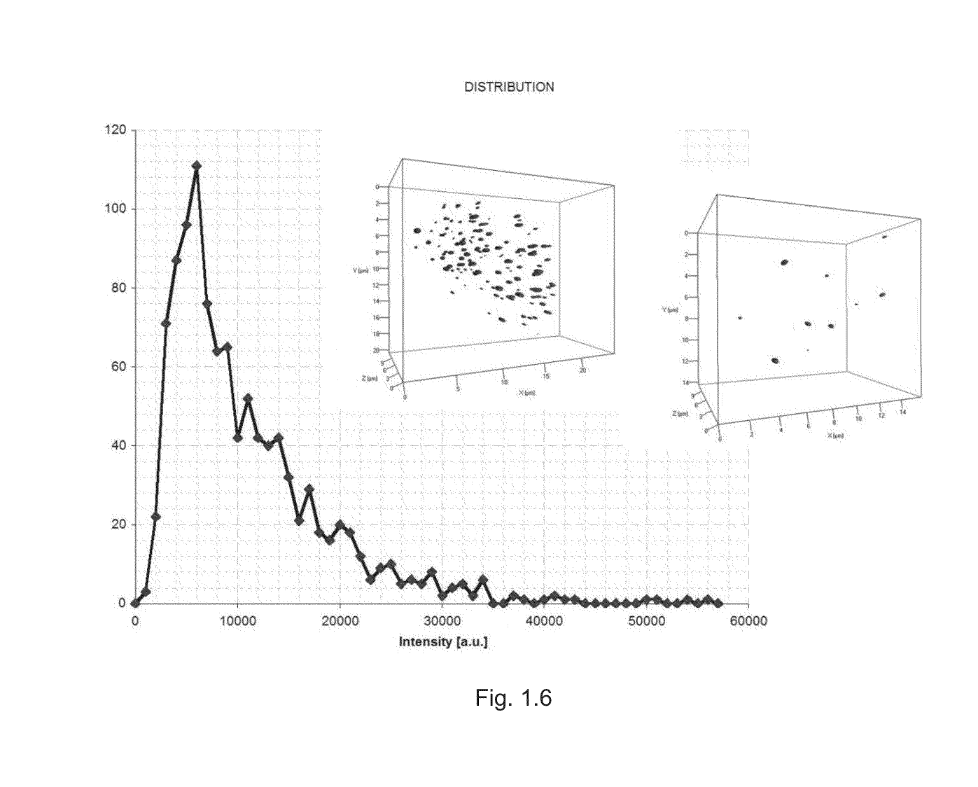

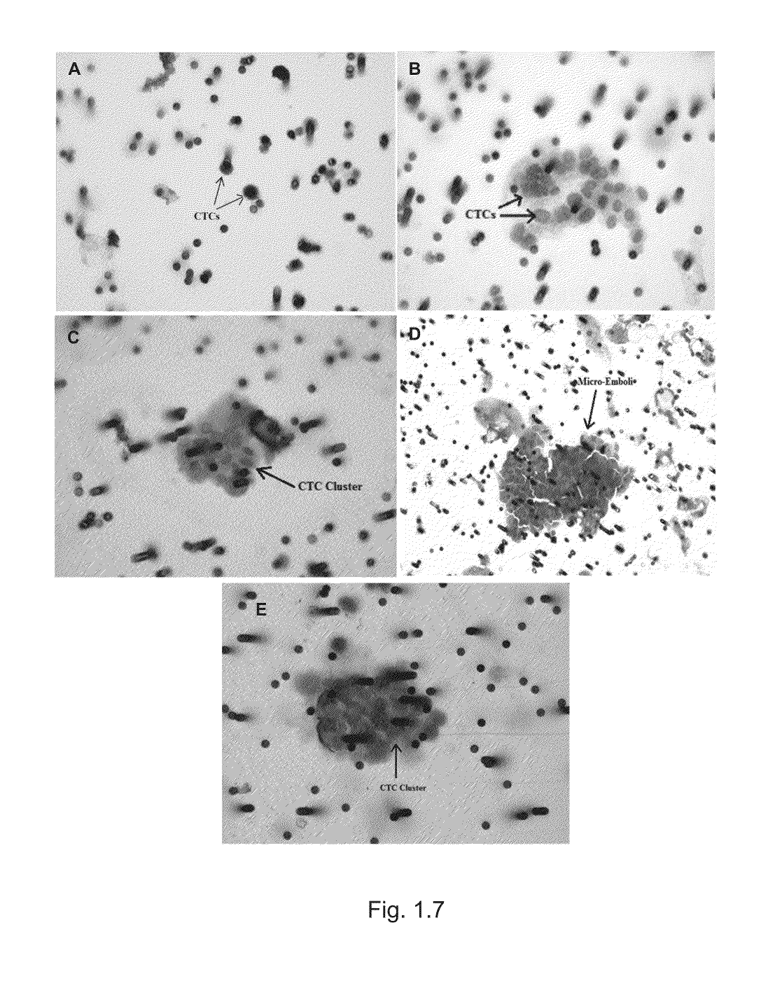

The telomere signatures can provide an indication of the characteristics such as aggressiveness of a subject cancer. For example, melanoma cells profiles as shown in FIG. 1.2 and FIG. 3D vary. The profile in FIG. 3D is characterized by a greater number of telomeres in the 40-60 000 unit range. Such differences indicate CTC heterogeneity and varying degrees of genomic instability in CTCs.

In an embodiment, the sub-population of CTCs is identified based on telomere number, telomere size and the presence and/or number of telomere aggregates. In an embodiment, the sub-population of CTCs is identified based on telomere size.

In an embodiment, 2, 3, 4, 5 or more subpopulations are identified, for example based on telomere size.

In yet a further embodiment, the method comprises identifying:

a. a first sub-population comprising CTCs with an average telomere intensity of less than about 40,000, less than about 30,000 less than about 20,000, less than about 15,000, less than about 10,000 or less than about 5,000 a.u.;

b. a second sub-population comprising CTCs with an average telomere intensity of about 5,000-40,000 to about 30,000-60,000 a.u.; and optionally

b. a third sub-population comprising CTCs with an average telomere intensity of more than about 25,000, more than about 30,000, more than about 40,000, more than about 50,000 or more than about 60,000 a.u.

The sub-populations can be identified according to the a.u. corresponding to different peaks or groups of peaks. The sup-populations can be identified by any range of a.u. depending for example on the cancer.

Additional subpopulations may be identified, for example 4 or more. Fewer subpopulations may be identified, for example 2 or one.

The subpopulations for example can provide information indicative of cancer heterogeneity, cancer burden, aggressiveness and/or stage.

In an embodiment, the method comprises identifying:

a. a first sub-population comprising CTCs with an average telomere intensity of less than about 20,000, less than about 25,000, less than about 30,000, less than about 35,000 or less than about 40,000 a.u.; and optionally

b. a second sub-population comprising CTCs with an average telomere intensity of more than about 25,000, more than about 30,000, more than about 35,000 or more than about 40,000 a.u.

Different average telomeres can define the populations of different cancers. In an embodiment, the tumor cell is:

a. a prostate cancer cell, and wherein the method of comprises identifying;
i. a first sub-population comprising CTCs with an average telomere intensity of less than about 20,000 a.u.;
ii. a second sub-population comprising CTCs with an average telomere intensity of about 20,000 to about 60,000 a.u.; and/or
iii. a third sub-population comprising CTCs with an average telomere intensity of more than about 50,000 a.u.; or

b. a colon cancer cell, and wherein the method of comprises identifying:
i. a first sub-population comprising CTCs with an average telomere intensity of less than about 10,000 a.u.;
ii. a second sub-population comprising CTCs with an average telomere intensity of about 10,000 to about 35,000 a.u.; and/or
iii. a third sub-population comprising CTCs with an average telomere intensity of more than about 35,000 a.u.; or

c. a breast cancer cell, and wherein the method of comprises identifying:
i. a first sub-population comprising CTCs with an average telomere intensity of less than about 10,000 a.u.;
ii. a second sub-population comprising CTCs with an average telomere intensity of about 10,000 to about 40,000 a.u.; and/or
iii. a third sub-population comprising CTCs with an average telomere intensity of more than about 40,000 a.u.; or

d. a melanoma cancer cell, and wherein the method of comprises identifying:
i. a first sub-population comprising CTCs with an average telomere intensity of less than about 20,000 to about 40,000 a.u.;
ii. a second sub-population comprising CTCs with an average telomere intensity of about 20,000-40,000 a.u. to about 40,000-60,000 a.u.; and/or
iii. a third sub-population comprising CTCs with an average telomere intensity of more than about 40,000 a.u. or more than about 60,000 a.u; or

e. a lung cancer cell, and wherein the method of comprises identifying:
i. a first sub-population comprising CTCs with an average telomere intensity of less than about 10,000 a.u.;
ii. a second sub-population comprising CTCs with an average telomere intensity of about 10,000 to about 30,000 a.u.; and/or
iii. a third sub-population comprising CTCs with an average telomere intensity of more than about 30,000 a.u.

The a.u. for defining a subpopulation boundary can be any whole number between for example 1 and 60,000 au. and can be selected by visually inspecting a plot and/or on the basis of maximizing similarities within a group or other criteria.

In an embodiment, the method further comprises isolating the sub-population.

A further aspect includes a method for identifying CTC subpopulations, the method comprising:

a. obtaining a plurality of 3D telomere organization signature datasets, each dataset corresponding to a unique isolated CTC;

b. determining for each dataset and/or a combination thereof, values for features from the 3D telomere organization signature datasets; and
i. identifying the subpopulation characteristics and/or the number of subpopulations based on one of more feature values.

In an embodiment, the features comprise at least one of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

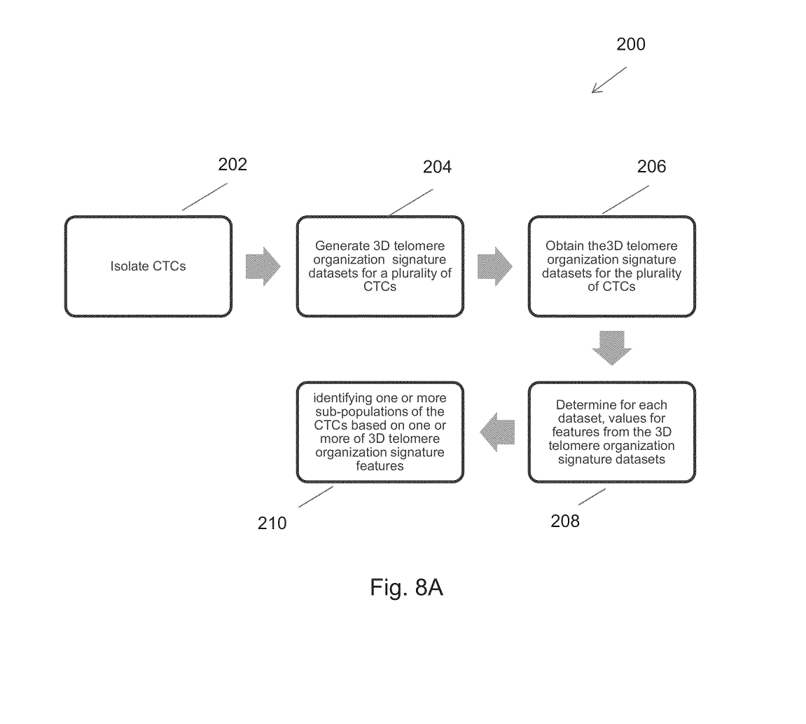

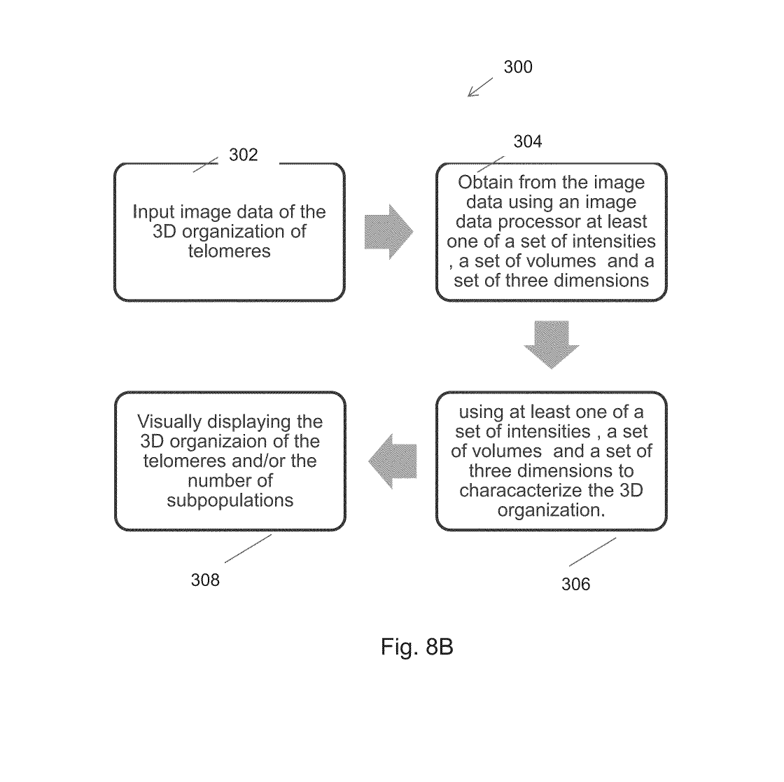

In an embodiment, the method of identifying one or more circulating tumour cell (CTC) subpopulations comprises a method depicted in FIG. 8A comprising:
a. isolating CTCs from a blood sample from a subject 202;
b. generating 3D telomere organization signature datasets for a plurality of CTCs 204;
c. obtaining the 3D telomere organization signature datasets for the plurality of CTCs 206;
d. determining for each dataset, values for features from the 3D telomere organization signature datasets 208; and
e. identifying one or more sub-populations of the CTCs based on one or more of 3D telomere organization signature features 210 selected from telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In a further embodiment, the features comprise at least one of telomere numbers, telomere intensities and telomeric aggregate numbers

In an embodiment, the plurality of 3D telomere organization signature datasets comprises at least 25, at least 30 or at least 40 datasets.

In an embodiment, the number of subpopulations is assessed. For example, as described below, a prostate cancer patient with 3 definable subpopulations of CTCs had advanced disease which was more aggressive than a subject with prostate cancer with 2 definable subpopulations of CTCs.

The subpopulations and/or their boundaries can be determined for example by visually inspecting the telomere intensity traces. The boundaries can also be determined based on statistical parameters. For example, the subpopulations can be defined as described in Knecht et al., 2009 Leukemia. Subpopulations for example are defined by comparison of telomere numbers, sizes, nuclear volumes, telomere distribution within the nucleus and/or nuclear sizes.

In an embodiment, each 3D telomere organization signature dataset is obtained using a method comprising:

a. isolating a plurality of CTCs from a blood sample from a subject; and

b. determining the 3D telomere organization signature of each of the plurality of isolated CTCs.

Another aspect includes an isolated sub-population of circulating tumour cells (CTCs) obtained by:

a. isolating a population of CTCs from the blood of a subject;

b. determining the 3D telomeres organization signature of the population of CTCs;

c. isolating a sub-population of the CTCs based on one more of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

For example, the CTCs could be isolated by microdissection from the filter devise and examined by PCR, sequencing and any other method.

In an embodiment, the isolated sub-population comprises CTCs with an average telomere intensity of less than about 40,000, less than about 20,000, less than about 15,000, less than about 10,000 or less than about 5,000 a.u.

In an embodiment, the sub-population comprises CTCs with an average telomere intensity of about 5,000-40,000 to about 30,000-60,000 a.u.

In yet another embodiment, the sub-population comprises CTCs with an average telomere intensity of more than about 20,000, more than about 25,000, more than about 30,000, more than about 40,000, more than about 50,000 or more than about 60,000 a.u. The sub-population can comprise CTCs with an average telomere intensity of any whole number between for example 1 and 60,000 a.u.

In yet another embodiment, the sub-population comprises CTCs with an average telomere intensity of more than about 20,000, 25,000, more than about 30,000, more than about 35,000 or more than about 40,000 a.u. or less than about 20,000, less than about 25,000, less than about 30,000, less than about 35,000 or less than about 40,000 a.u.

In an embodiment CTCs are compared to other control circulating cells such as lymphocytes. A subject's lymphocytes can be used an internal control for assessing the CTC genomic profile's (e.g. the extent of variation). As demonstrated herein, the genomic profile of CTCs varies dramatically compared to lymphocytes. Lymphocytes or other normal circulating cells can provide an internal control when for example comparing from sample to another or comparing to a population control.

Since telomere length is age-dependent, the use of lymphocytes from the same patient enables assessment of a subject's age-dependent telomere profile. In addition, a comparison/ratio of all telomere parameters can be made for the purpose of establishing a patient-specific index of genomic instability in CTCs.

CTCs can be distinguished from other circulating cells on the basis of cell size, nuclear volume, variations in telomere signal intensity, and/or telomere numbers.

A further aspect includes an assay comprising:
a. determining a 3D telomeres organization signature for a plurality of isolated test CTCs isolated from a blood sample from a subject with cancer;
b. identifying one or more sub-populations according to a method described herein; and
c. comparing the 3D telomeres organization signature of the test CTC subpopulations with a reference 3D telomeres organization signature, and if there is a difference or similarity in the 3D telomeres organization signature of the test CTCs and the reference 3D telomeres organization signature, identifying the subject as having an increased probability of a positive or negative clinical outcome.

In an embodiment, the reference 3D telomeres organization signature is a subject base-line level, for monitoring a subject. Changes indicative of more aggressive disease indicate that the subject is progressing, and/or if on treatment, not responding to treatment. Changes indicative of less aggressive disease indicate that the subject is not progressing, and/or if receiving treatment, responding to the therapy.

In an embodiment, the clinical outcome is progression.

In another embodiment, the clinical outcome is recurrence.

In yet another embodiment, the 3D telomeres organization signature comprises one or more of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In yet another embodiment, the 3D telomeres organization signature comprises one or more of telomere numbers, telomere size and number of aggregates, and wherein an aberrant number of telomere, a decrease in average telomere size and/or an increased number of aggregates in the 3D telomeres organization signature of one or more subpopulations of the test CTCs is indicative of an increased probability of a negative clinical outcome.

In an embodiment, the presence of telomere aggregates in at least 35%, at least 40%, at least 45%, at least 50%, at least 55%, at least 60%, at least 70% or at least 80% of one or more subpopulations of the test CTCs is indicative of an increased probability of a negative clinical outcome.

In an embodiment, the assay further comprises identifying the number of CTCs in the blood sample and wherein more than about 25, more than about 30, more than about 35, more than about 40, more than about 45, more than about 50, more than about 60, more than about 70 or more than about 80 CTCs in 3.5 mL of blood is indicative of an increased probability of a negative clinical outcome.

It has been suggested that <5 CTCs can be indicative of a ‘good’ prognosis and >5 CTCs can be indicative of a poor prognosis. Additional prognostic information can be achieved by assessing the genomic characteristics of such cell. Further, the number of CTCs isolated can vary due to their rare presence per mL of blood. Accordingly, in an embodiment, the aggressiveness of the CTC as indicated at least in part by the 3D profile of each CTC is assessed to provide a prognosis.

For example, a threshold of <5 CTCs/10⁹ blood cells can be applied as a marker for good/stable disease and >5 CTCs/10⁹ blood cells for poor/aggressive disease (Danila et al., 2010) to establish two groups of patients. However stable disease does not mean there is no risk of progression and the risk can be assessed for example, by characterizing the telomeric organization of subpopulations (such as aggressive, stable). For example, 4 aggressive CTCs can be more critical than 6 non-aggressive CTCs. In an embodiment, the population of test CTCs is organized into sub-populations based on telomere size and more than 2, 3, 4 or 5 sub-populations is indicative of an increased probability of a negative clinical outcome.

Yet another aspect includes a method of prognosing a clinical outcome in a subject with cancer comprising:
a. isolating CTCs from a blood sample from the subject to obtaining test sample CTCs, and
b. determining a 3D telomere organization signature of the test sample CTCs using 3D q-FISH;
c. identifying a likelihood of a clinical outcome based on the 3D telomere organization signature of the test sample CTCs.

In an embodiment, the CTCs are isolated from the blood sample using a filter device.

In another embodiment, comparing the 3D telomere organization signature of the test sample CTC subpopulations with a 3D telomere organization signature in a control, wherein a difference or similarity in the 3D telomere organization signature(s) between the test sample CTC subpopulations and the control is indicative of the clinical outcome of the subject.

In an embodiment, the cancer is melanoma, colorectal cancer, lung cancer, breast cancer or prostate cancer.

In yet another embodiment, the 3D telomere organization signature comprises one or more of telomere number, telomere size, presence and/or number of telomeric aggregates, percentage of cells with telomeric aggregates, average number of telomeric aggregates per cell, average number of telomeres per cell, telomeres per nuclear volume, distances from nuclear centre, average nuclear volume and a/c ratio.

In an embodiment, the 3D telomeres organization signature comprises one or more of telomere numbers, telomere size and number of aggregates, and wherein an aberrant number of telomere, a decrease in average telomere size and/or an increased number of aggregates in the 3D telomeres organization signature of the test CTCs is indicative of an increased probability of a negative clinical outcome.

In an embodiment, wherein the presence of telomere aggregates in at least 35%, 40%, 45%, 50%, 55%, 60%, 70% or 80% of the test CTCs in one or more sub-populations is indicative of an increased probability of a negative clinical outcome.

In an embodiment, the assay further comprises identifying the number of CTCs in the blood sample and wherein more than about 25, more than about 30, more than about 35, more than about 40, more than about 45, more than about 50, more than about 60, more than about 70 or more than about 80 CTCs in about 3.5 mL of blood is indicative of an increased probability of a negative clinical outcome.

A further aspect includes a population of test CTCs organized into sub-populations based on telomere size and more than 2, 3, 4 or 5 sub-populations is indicative of an increased probability of a negative clinical outcome.

It is demonstrated herein that microemboli can be analyzed using the methods described herein. Accordingly, the number of microemboli is in an embodiment, measured and used in conjunction with the CTC 3D telomere profile to provide clinical information of the subject's disease.

In an embodiment, the method further comprises assessing the number of circulating tumour microemboli, wherein an increased number of circulating tumour microemboli is a poor prognosticator.

A method of treating a subject is also provided in an embodiment, comprising:
a. identifying one or more CTC sub-populations in a sample from the subject;
b. assessing one or more characteristics of the CTC subpopulations, optionally by comparing to one or more reference 3D telomeres organization signatures, and
c. providing a suitable treatment according to the characteristics of the CTC sub-populations.

For example, if the sample is characterized with one or more sub-populations that have characteristics of an aggressive cancer, a suitable treatment can be provided. If the sample is characterized with one or more sub-populations that have characteristics of a non-aggressive cancer, optionally the subject is monitored and/or a less toxic treatment is provided.

In an embodiment, the method of treatment comprises prognosing the clinical outcome of a subject according to the method described herein and providing a suitable treatment according to the prognosis. The suitable treatment can be no treatment if the subject is not progressing or an accepted treatment for the subject's cancer.

### 3D Image Acquisition and Analysis

In an embodiment, the 3D telomeric organization signature is determined using 3D quantitative FISH (3D q-FISH).

The 3D images can be obtained using a 3D imaging system that enables Abbe resolution of 200 nm, for example an AxioIMager Z2 (Zeiss) microscope.

The In an embodiment, the method uses TeloScan™. In another embodiment, the method uses TeloView™. For example, both TeloScan™ and TeloView™ can be used to determine the 3D telomere organization of a cell. TeloScan™ is capable of scanning multiple cells at one time; whereas TeloView™ scans one cell at a time. Further details on these methods follow.

Telomere Q-FISH: The telomere FISH protocol was performed by using Cy3-labelled peptide nucleic acid (PNA) probes (DAKO). Imaging of interphases after telomere FISH was performed by using Zeiss AxioImager Z1 with a cooled AxioCam HR B&W, DAPI, Cy3 filters in combination with a Planapo 63x/1.4 oil objective lens. Images were acquired by using AXIOVISION 4.6 and 4.8 (Zeiss) in multichannel mode followed by constraint iterative deconvolution as specified below.

3D Image Acquisition: At least 30 H-cell interphase nuclei and 30 RS-cell interphase polycaria were analyzed in each lymph node slide. AXIOVISION 4.6 and 4.8 with deconvolution module and rendering module were used. For every fluorochrome, the 3D image consists of a stack of 40 images with a sampling distance of 200 nm along the z and 107 nm in the x and y direction. The constraint iterative algorithm option was used for deconvolution.

3D Image Analysis for Telomeres: Telomere measurements were done with TeloView™. By choosing a simple threshold for the telomeres, a binary image is found. Based on that, the center of gravity of intensities is calculated for every object resulting in a set of coordinates (x, y, z) denoted by crosses on the screen. The integrated intensity of each telomere is calculated because it is proportional to the telomere length.

Statistical analysis: For each case, normally distributed parameters are compared between the two types of cells using nested ANOVA or two-way ANOVA. Multiple comparisons using the least square means tests followed where interaction effects between two factors were found to be significant. Other parameters that were not normally distributed were compared using a nonparametric Wilcoxon rank sum test. Significance level were set at p=0.05. Analyses were done using SAS v9.1 programs.

Further details of the method of characterizing 3D telomere organization follows. In an embodiment the method for characterizing a 3D organization of telomeres comprises:

(i) inputting image data of the 3D organization of telomeres;

(ii) processing the image data using an image data processor to find a set of coordinates {(xᵢ, yᵢ, zᵢ)}, i=1, . . . , N, where (xᵢ, yᵢ, zᵢ) is a position of the ith telomere;

(iii) finding a plane that is closest to the set of coordinates; and

(iv) finding a set of distances {dᵢ}, i=1, . . . , N, where dᵢ is the distance between (xᵢ, yᵢ, zᵢ) and the plane, wherein the set {dᵢ} is utilized to characterize the 3D organization.

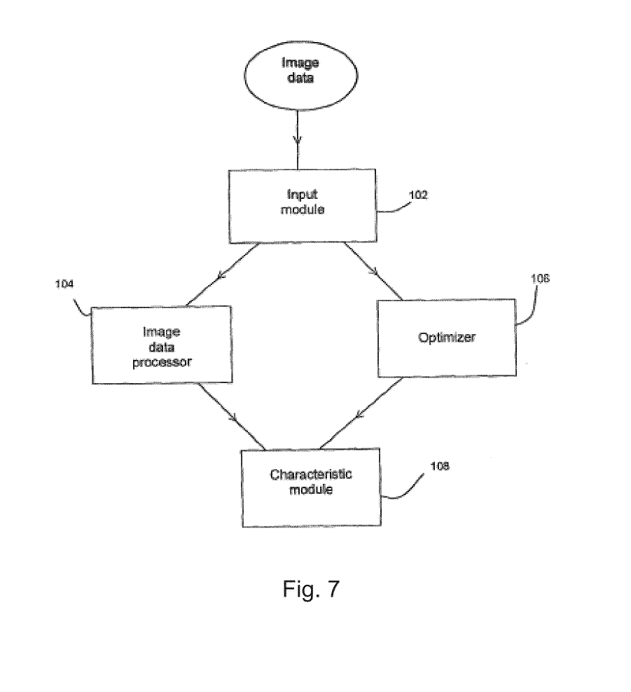

FIG. 7 shows a block diagram of a system 100 for characterizing a 3D organization of telomeres. The system 100 includes an input module 102, an image data processor 104, an optimizer 106 and a characteristic module 108.

An input module 102 can be used to input image data of the 3D organization of telomeres. The input module 102 includes appropriate hardware and/or software, such as a CD-ROM and CD-ROM reader, DVD and DVDreader or other data storage and reading means including for example external hard drives. The inputting performed by the input module 102 need not be from outside the system 100 to inside the system 100. Rather, in some embodiments, the inputting of data may describe the transfer of data from a permanent storage medium within the system 100, such as a hard disk of the system 100, to a volatile storage medium of the system 100, such as RAM.

The image data can be obtained using regular or confocal microscopy and can include the intensities of one or more colors at pixels (totaling, for example, 300×300 or 500×500) that comprise an image of a nucleus. The image data can also be grey level image data of a nucleus that has been appropriately stained to highlight telomeres. Several images (on the order of 100) are obtained corresponding to slices along a particular axis. Thus, the image data may correspond to a total of about 2.5×10′ pixels. In one embodiment, the slices may be on the order of 100 nanometers apart. In this manner, the image data accounts for the 3D quality of the organization of telomeres. In addition, the confocal microscope is able to obtain the intensity of two colors, for example blue and green, of the nucleus at every pixel imaged, thereby doubling the amount of data points.

To obtain an image of telomeres, a stain such as DAPI (4′,6-diamidino-2-phenylindole) can be used to preferentially mark the heterochromatin material that comprises DNA. A second stain, such as cy3, together with an appropriate label, such as PNA telomere probe, can be used to mark the telomeric portion of the heterochromatin material.

To improve the quality of the image data, various techniques can be brought to bear as known to those of ordinary skill, such as constrained iterative deconvolution of the image data to improve resolution. Such constrained iterative deconvolution may not be required if confocal, instead of regular, microscopy is used as the image data may be of superior resolution. In addition, other instruments, such as an apotome, may be used to improve the quality of the image.

In an embodiment, the 3D organization is characterized by specifying at least one of d̅ and σ, where d̅ is the average distance of the set of distances, and σ is the standard deviation of the set of distances.

In another embodiment, the characterization is used to monitor and/or diagnose cancer disease by comparing the at least one of d̅ and σ for each subpopulation to a corresponding control value and/or other subpopulations.

In an embodiment, the method of characterizing a 3D organization of telomeres comprises:

(i) inputting image data of the 3D organization of telomeres; and

(ii) using an image data processor for finding a three dimensional geometrical shape that best encompasses the 3D organization, wherein the geometrical shape is an ellipsoid having principal axes a₁, a₂, and a₃ and wherein said shape is used to characterize the 3D organization.

The image data processor 104 processes the image data to find a set of coordinates {(xᵢ, yᵢ, zᵢ)}, i=1, . . . , N, where (xᵢ, yᵢ, zᵢ) is a position of the ith telomere. For this purpose, the image data processor 104 identifies “blobs” within the image data that can be identified as a telomere using a segmentation process. Each blob identified as a telomere has a non-negligible volume (for example, a small telomere may have a volume of 4×4×4 pixels, a large one a volume of 10×10×10, where the size of the nucleus may be approximately 200×200×100 pixels). There is some freedom, therefore, in choosing “the position” of the telomere. One possibility is to choose for this position the center of gravity of the telomere, or more generally, the telomere organization.

In an embodiment, the ellipsoid is an oblate spheroid with a₁ approximately equal to a₂.

In an embodiment, an oblateness ratio, a₃/a₁ or a₁/a₃, is used to characterize the 3D organization.

In an embodiment, the method for characterizing a 3D organization of telomeres comprises:

(i) inputting image data of the 3D organization of telomeres and

(ii) obtaining from the image data using an image data processor at least one of a set of intensities {Iᵢ}, a set of volumes {Vᵢ} and a set of three dimensions {(Dxᵢ, Dyᵢ, Dzᵢ)}, where i=1, . . . , N, where Iᵢ is a total or average intensity, Vᵢ is a volume, and (Dxᵢ, Dyᵢ, Dzᵢ) are principle axes of an ellipsoid describing the ith telomere, respectively, wherein the at least one is utilized to characterize the 3D organization.

In an embodiment, the quantity is an average of the members of {Iᵢ}, {Vᵢ} or (Dxᵢ, Dyᵢ, Dzᵢ).

In an embodiment, the method for characterizing a 3D organization of telomeres comprises:

(i) obtaining image data of the 3D organization of telomeres obtained using a microscope;

(ii) inputting the image data of the 3D organization of telomeres obtained using the microscope; and

(iii) finding a parameter of the 3D organization that measures a deviation of the 3D organization from a planar arrangement, the deviation used to characterize the 3D organization.

In yet another embodiment, the method for characterizing a 3D organization of telomeres of sample cells comprises:

(i) obtaining image data of the 3D organization of telomeres obtained using a microscope;

(ii) inputting the image data of the 3D organization of telomeres;

(iii) processing the image data to find a set of coordinates {(xᵢ, yᵢ, zᵢ)}, i=1, . . . , N, where (xᵢ, yᵢ, zᵢ) is a position of the ith telomere;

(iv) finding a plane that is closest to the set of coordinates;

(v) finding a set of distances {dᵢ}, i=1, . . . , N, where dᵢ is the distance between (xᵢ, yᵢ, zᵢ) and the plane, wherein the set {fᵢ} is utilized to characterize the 3D organization; and

(vi) visually displaying the 3D organization of the telomeres.

In an embodiment, the method for characterizing a 3D organization of telomeres of sample cells is performed on a system for characterizing a 3D organization of telomeres.

In an embodiment, the system comprises:

(i) an input module for inputting image data of the 3D organization of telomeres;

(ii) an image data processor for processing the image data to find a set of coordinates {(xᵢ, yᵢ, zᵢ)}, i=1, . . . , N, where (xᵢ, yᵢ, zᵢ) is a position of the ith telomere;

(iii) an optimizer for finding a plane that is closest to the set of coordinates; and

(iv) a characteristic module for finding a set of distances {dᵢ}, i=1, . . . , N, where dᵢ is the distance between (xᵢ, yᵢ, zᵢ) and the plane, wherein the set {dᵢ} is utilized to characterize the 3D organization.

The optimizer 106 finds a plane P^{min} that is closest to the set of coordinates. To find the closest plane, the distance Dᵢ between the location of the ith telomere, (xᵢ, yᵢ, zᵢ), and the plane given by ax+by+cz=0 is considered:

$${D}_{i} = \frac{{ax}_{i} + {by}_{i} + {cz}_{i}}{\sqrt{{a}^{2} + {b}^{2} + {c}^{2}} } .$$

The optimizer 106 finds the parameters a, b, c, d that minimize the function

$${\sum }_{i = 1}^{N} {D}_{i} ( a , b , c , d ) .$$

The characteristic module 108 proceeds to find at least one parameter that can be used to characterize the 3D organization of telomeres. “Parameters used to characterize the organization of telomeres” include:

1) A set of distances {dᵢ}, i=1, . . . , N, where dᵢ is the distance between (xᵢ, yᵢ, zᵢ) and the plane P^{min}.

2) d̅ and σ, the average distance and standard deviation of the set of distances {dᵢ}:

$$\overline{d} = \frac{1}{N} {\sum }_{i = 1}^{N} {d}_{i} , \\ and$$

$${σ}^{2} = {\sum }_{i = 1}^{N} \frac{{( {d}_{i} - \overline{d} )}^{2}}{N} ,$$

respectively.

3) A three dimensional geometrical shape that best encompasses the 3D organization. For example, the geometrical shape can be the ellipsoid, having principal axes a₁, a₂, and a₃, that best encompasses the 3D organization of the telomeres. Several definitions of “best encompasses” can be used. For example, the ellipsoid that best encompasses the telomeres can be defined as the ellipsoid of smallest volume that encloses a certain fraction (e.g., 100%) of the telomeres. If a set of more than one ellipsoid fulfills this condition, other restrictions can be used to reduce the set to just one ellipsoid, such as further requiring the ellipsoid to have the smallest largest ratio of principle axes (i.e., the “most circle-like” ellipsoid). It should be understood that other definitions of “best encompasses” the telomeres can be used. It has been observed that the ellipsoid that best encompasses the telomeres often approximates an oblate spheroid with a₁ approximately equal to a₂. In such case, it is sufficient to specify just a₂ and a₃. Alternatively, an oblateness ratio, a₃/a₁ or a₁/a₃, can be used to characterize the oblate spheroid describing the organization of the telomeres.

4) A set of volumes {Vᵢ}, where Vᵢ is the volume of the ith telomere.

5) A set of three dimensions {(Dxᵢ, Dyᵢ, Dzᵢ)}, i=1, . . . , N, where (Dxᵢ, Dyᵢ, Dzᵢ) are principle axes of an ellipsoid describing the ith telomere.

6) A set of intensities {Iᵢ}, i=1, . . . , N, where Iᵢ is the total intensity of the ith telomere. (In other embodiments, instead of the total intensity, the average intensity of each telomere can be computed.) That is, if the ith telomere is associated with K pixels, then

$${I}_{i} = {\sum }_{j = 1}^{K} {I}_{i , j}$$

where I_{i,j} is the intensity of the jth pixel of the ith telomere.

In the last three cases, the sets can be used to calculate statistical measures such as an average, a median or a standard deviation.

The parameters 1-5 outlined above characterize the 3D organization of the telomeres by focusing on the geometrical structure of the telomeres. Parameters 1 and 2 are motivated by the finding that, especially during the late G2 phase of the cell cycle, telomeres tend to lie on a plane. Parameters 1 and 2 measure deviations of telomeres from a planar arrangement.

Parameter 3 attempts to describe, with features, such as the three principal axes of an ellipsoid or the oblateness ratio, the overall shape of the 3D organization. While parameters 1-3 are global geometric characteristics, dealing with the overall shape of the organization, parameters 4 and 5 are local geometric characteristics in the sense that they involve the geometry of each individual telomere.

The final parameter is also local, involving the intensity of each individual telomere.

In an embodiment, the 3D organization is characterized by specifying at least one of d̅ and σ, where d̅ is the average distance of the set of distances, and σ is the standard deviation of the set of distances.

In an embodiment, the system further comprises a diagnosis module for comparing the at least one of d̅ and σ to a corresponding standard value to compare subpopulations, for example the number of subpopulations between samples.

In another embodiment, the method for characterizing a 3D organization of telomeres in the sample comprises:

(i) inputting image data of the 3D organization of telomeres; and

(ii) using an image data processor for finding a parameter of the 3D organization that measures a deviation of the 3D organization from a planar arrangement, the deviation used to characterize the 3D organization.

In an embodiment, a system is used for characterizing a 3D organization of telomeres in the sample, the system comprising

(i) an input module for inputting image data of the 3D organization of telomeres;

(ii) an image data processor for processing the image data to find a set of coordinates {(xᵢ, yᵢ, zᵢ)}, i=1, . . . , N, where (xᵢ, yᵢ, zᵢ) is a position of the ith telomere; and

(iii) a characteristic module for finding a parameter of the distribution that measures a deviation of the distribution from a planar arrangement, the deviation used to characterize the 3D organization.

In an embodiment, the method for characterizing a 3D organization of telomeres comprises:

(i) obtaining image data of the 3D organization of telomeres obtained using a microscope;

(ii) inputting the image data of the 3D organization of telomeres obtained using the microscope;

(iii) processing the image data to find a set of coordinates {(xᵢ, yᵢ, zᵢ)}, where (xᵢ, yᵢ, zᵢ) is a position of the ith telomere;

(iv) finding a plane that is closest to the set of coordinates; and

(v) finding a set of distances {dᵢ}, i=1, . . . , N, where dᵢ is the distance between (xᵢ, yᵢ, zᵢ) and the plane, wherein the set {dᵢ} is utilized to characterize the 3D organization.

In another embodiment, the method of characterizing a 3D organization of telomeres, comprises:

(i) obtaining image data of the 3D organization of telomeres obtained using a microscope;

(ii) inputting the image data of the 3D organization of telomeres obtained using the microscope; and

(iii) finding a three dimensional geometrical shape that best encompasses the 3D organization, wherein the geometrical shape is an ellipsoid having principal axes a₁, a₂, a₃ and wherein said shape is used to characterize the 3D organization.

In another embodiment, the method for characterizing a 3D organization of telomeres, comprises:

(i) obtaining image data of the 3D organization of telomeres obtained using a microscope;

(ii) inputting the image data of the 3D organization of telomeres obtained using the microscope; and

(iii) obtaining from the image data at least one of a set of intensities {Iᵢ}, a set of volumes {Vᵢ} and a set of three dimensions {(Dxᵢ, Dyᵢ, Dzᵢ)}, i=1, . . . , N, where Iᵢ is a total or average intensity, Vᵢ is a volume, and (Dxᵢ. Dyᵢ, Dzᵢ) are principle axes of an ellipsoid describing the ith telomere, respectively, wherein the at least one is utilized to characterize the 3D organization.

In an embodiment, determining the 3D organization of telomeres in CTC subpopulations and optionally comparing to a control is a computer implemented method.

In an embodiment, the computer implemented method is TeloVew. In another embodiment, the computer implemented method is TeloScan™.

Further, the definitions and embodiments described are intended to be applicable to other embodiments herein described for which they are suitable as would be understood by a person skilled in the art. For example, in the above passages, different aspects of the disclosure are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### EXAMPLES

### Example 1

### Isolation and Characterization of CTC Cells

CTCs from blood of patients with non-small cell lung carcinoma, melanoma, breast cancer and colon cancer were isolated using a ScreenCell filter device according to protocols and methods described in Desitter E et al., AntiCancer Research 31: 427-422 (2011). The ScreenCell filter device is shown to allow for example an average recovery of about 91.2% (assessed by spiking 5 cells in a 1 mL of blood). Cells spiked into whole blood and isolated using the ScreenCell device can by lysed and RNA can be extracted directly from cells on the filter. As shown in Desitter et al, the SreenCell Cyto device allows isolation of CTCs from peripheral blood of a patient for example with non-small cell lung carcinoma. Micro emboli can also be isolated from blood for example of a patient with melanoma or colon cancer

The cells captured on the filter were 3D fixed (Louis et al., 2005). The 3D nuclear organization of the telomeres within the nuclei of captured cells was analyzed as follows: 3D quantitative fluorescent in situ hybridization (Q-FISH) was performed as published (Louis et al., 2005) using a Cy3-labelled peptide nucleic acid (PNA) probe (DAKO). The nuclei were counterstained with 4′-6-diamidino-2-phenylindole (DAPI). 5 μm sections of paraffin-embedded tissue biopsies were deparaffinized using xylene and then rehydrated and analyzed for 3D nuclear telomere organization.

Imaging and analysis utilized the programs TeloView™ (Vermolen et al., 2005; Gonzalez-Suarez, 2009) and TeloScan™ (Gadji et al., 2010; Klewes et al., 2011). For TeloView™ analysis (Vermolen et al., 2005; Gonzalez-Suarez, 2009), imaging of nuclei was performed by using Zeiss AxioImager Z2 with a cooled AxioCam HR B&W, DAPI, Cy3 filters in combination with a Planapo 63x/1.4 oil objective lens. Images were acquired by using AXIOVISION 4.8 (Zeiss) in multichannel mode followed by constrained iterative deconvolution (Schaefer et al., 2001). For every fluorochrome, image stacks were acquired with a sampling distance of 200 nm along the z and 107 nm in the xy direction. TeloScan, the automated version of TeloView, was performed on a scanning platform, the SpotScan system (Applied Spectral Imaging, Migdal HaEmek, Israel). The system uses an automated Olympus BX61 microscope (Olympus, Center Valley, Pa.) equipped with filters for DAPI and Cy3. Using images of 13 focal planes 0.7 μm apart, TeloScan was used to scan in telomeres in 3D and store all 3D datasets (Klewes et al., 2011).

The results of the 3D telomere analysis is shown for CTC cells isolated from patients with non small cell lung carcinoma (FIG. 1.1), melanoma (FIG. 1.2), breast cancer (FIGS. 1.3-1.5) and colon cancer (FIG. 1.6).

### Example 2

### Isolation and Characterization of CTC Cells

### Patients

Ten filtered patient samples and one lung cancer cell line were analysed. An additional nine prostate cancer samples were obtained. The patient population consists of nine prostate cancer, one colon cancer, three breast cancer, and six melanoma cases and one lung cancer cell line (Table 1).

There was no prior knowledge of the clinical data of the patients involved to enable an unbiased analysis of the samples. The classification of patients into stages of cancer was done blindly on the basis of the 3D profiles of telomeres observed in their CTCs and was confirmed with clinical parameters in post hoc fashion.

TABLE 1
Clinical Data of the Patients Who Participated in the Study.
(TRUS, transrectal ultrasound; PSA, prostate-specific antigen; PR, prostate; MB, Manitoba;
MM, malignant melanoma; SSM, superficial spreading melanoma.)
Clinical Data of the Patients Who Participated in the Study.

| | | Lifestyle | | |
|---|---|---|---|---|
| | Demography | Smoking/Pack | | Family History |
| Patient ID | Age/Ethnicity | Years/Year Quit | Alcohol/Frequency | Relation/Domain/Cancer |
| MB0181PR | 59 Caucasian | Ex-smoker | Occasional | Unknown |
| MB0182PR | 73 Black | Unknown | | |
| MB0189PR | 66 Caucasian | Ex- | Occasional | Brother/immediate/prostate |
| | | smoker/unknown/40 | | cancer |
| | | | | Sister/immediate/kidney |
| | | | | cancer |
| MB0211PR | 62 Caucasian | Nonsmoker | Daily | Grandfather/unknown/prostate |
| | | | | cancer |
| | | | | Father/immediate/lung cancer |
| MB0213PR | 50 Caucasian | Smoker/N/A | Never | Father/immediate/prostate |
| | | | | cancer |
| | | | | Grandfather/paternal/prostate |
| | | | | cancer |
| MB0216PR | 65 Caucasian | Nonsmoker | Never | Father/immediate/prostate |
| | | | | cancer |
| MB0217PR | 57 Caucasian | Nonsmoker | Never | Mother/immediate/unknown |
| MB0222PR | 59 Caucasian | Ex-Smoker/15/12 | Weekly | Brother/immediate/prostate |
| | | | | cancer |
| | | | | Father/immediate/colon |
| | | | | cancer |
| MB0239PR | 60 Caucasian | Unknown | Weekly | Grandfather/maternal/prostate |
| | | | | cancer |
| | | | | Father/immediate/colon |
| | | | | cancer |
| Colon GUI | M/68 | Unknown | Unknown | Father/colon cancer |
| 2F, 3F, 5F | Caucasian | | | |
| BR MERT | F/30 | Unknown | Unknown | Mother/breast cancer |
| 10AA5083 | Caucasian | | | |
| BR MIC | F/82 | Unknown | Unknown | None |
| 10AA3956 | Caucasian | | | |
| 10AA3934 | | | | |
| BR WUR | F/79 | Unknown | Unknown | Brother/atypical nevus |
| 10AA2499 | Caucasian | | | |
| Mela GOD | F/45 | Unknown | Unknown | None |
| 10AA4991 | Caucasian | | | |
| Mela CAR | M/21 | Unknown | Unknown | Father/MM* |
| 10AA2213 | Caucasian | | | |
| Mela SAU | F/72 | Unknown | Unknown | None |
| 10AA2408 | Caucasian | | | |
| Mela ROB | M/78 | Unknown | Unknown | Unknown |
| 10AA2621 | Caucasian | | | |
| Mela GAU | M/45 | Unknown | Unknown | None |
| 10AA3836 | Caucasian | | | |
| Mela | F/80 | Unknown | Unknown | None |
| CHAN | Caucasian | | | |
| 10AA4280 | | | | |
| Lung CA | Cell line | Cell line | Cell line | Cell line |
| H2030 | | | | |

| | | Investigations and Management with | |
|---|---|---|---|
| | | Dates | |
| | | Pathology/Laboratory | Treatment with |
| Patient ID | Comorbidities | Findings | Dates |
| MB0181PR | None | PSA: 4.42 μg/l (June 2011); | TRUS (February 2012) |
| | | 5.95 μg/l (September 2011); 9.26 μg/l | |
| | | (January 2012). | |
| | | Gleason score N/A | |
| | | Small cell carcinoma | |
| MB0182PR | Hypertension | PSA: 9.51 μg/l (June 2011) | TRUS (August 2011) |
| MB0189PR | Paget's Dx | Adenocarcinoma | TRUS (2007-2011) |
| | Hypertension | | Radical |
| | Dysplasia | PSA: 4.46 to 7.49 μg/l | prostatectomy |
| | | (2007-2011) | with bilateral |
| | Glaucoma | Gleason: 6-7 (2007-2012) | pelvic |
| | | | lymphadenectomy |
| | | | (Mar. 12, 2012) |
| MB0211PR | Hypothyroid | Adenocarcinoma | TRUS |
| | | | (Mar. 15, 2012) |
| | | PSA: 4.55-6.04 μg/l | Radical |
| | | (2009-2012) | prostatectomy |
| | | Gleason: 7 (2012) | with bilateral |
| | | | pelvic |
| | | | lymphadenectomy |
| | | | (May 31, 2012) |
| MB0213PR | None | Adenocarcinoma | TRUS (Mar. 14, 2012) |
| | | PSA: 3.15 μg/l (2012) | |
| | | Gleason: 7 (2012) | |
| MB0216PR | None | Benign | TRUS (Jan. 4, 2011) |
| | | adenocarcinoma | |
| | | PSA: 5.6-1.52 μg/l | TRUS (Jun. 12, 2012) |
| | | (2008-2012) | |
| | | Gleason: 9 (2012) | |
| MB0217PR | None | Benign | Radical |
| | | adenocarcinoma | prostatectomy |
| | | PSA: 80.91 μg/l | with bilateral |
| | | (2007); 60.28 μg/l | pelvic |
| | | (2008); <0.01 μg/l | lymphadenectomy |
| | | (2012) | (Jun. 5, 2008) |
| | | Gleason: 7 (2008) | |
| MB0222PR | Orchitis | Adenocarcinoma | Docetaxel, 166.5 mg |
| | 1/1/1986 | | |
| | | PSA: 4.61 μg/l (June 2011); | Leuprolide, 22.5 mg |
| | | 3.73μg/l (212); <0.01 μg/l | |
| | | (June 2012) | |
| | | Gleason: 7 (July 2011); 8 | |
| | | (November 2011) | |
| MB0239PR | None | Adenocarcinoma | TRUS (14/6/2006) |
| | | PSA: 3.26 μg/l (April 2006); | |
| | | 8.81 μg/l (April 2012); 5.4 μg/l | |
| | | (July 2012) | |
| | | Gleason: 6 (June 2006) | |
| Colon GUI | Asthma | Colorectal | Tumor excision + |
| 2F, 3F, 5F | | adenocarcinoma | Ki-adenectomy |
| | | Ras mutation + | (25/6/2010) |
| BR MERT | None | Invasive lobular | Tumor excision |
| 10AA5083 | | AdenoK Erb2(−) | (Aug. 9, 2010) |
| BR MIC | None | Invasive Lobular | Tumor excision |
| 10AA3956 | | adenoK Erb2(−) | (22/9/2010) |
| 10AA3934 | | | |
| BR WUR | Atypical nevus | Invasive lobular | Tumor excision |
| 10AA2499 | | AdenoK Erb2(−) | (Aug. 9, 2010) |
| Mela GOD | Benign nevus | SSM* grade 3; | Tumor excision |
| 10AA4991 | | thickness, 0.7 mm | (30/7/2010) |
| Mela CAR | None | SSM* grade 3; | Tumor excision |
| 10AA2213 | | thickness, 1.4 mm | (Jan. 7, 2010) |
| Mela SAU | Basal cell | Nodular MM* grade 4; | Tumor excision |
| 10AA2408 | carcinoma | thickness, 7 mm | (23/7/2010) |
| Mela ROB | Benign nevus | SSM* grade 4; | Tumor excision |
| 10AA2621 | | thickness, 4 mm, | (28/6/2010) |
| | Diabetes | | |
| Mela GAU | Basal cell | Nodular MM* grade 4; | Tumor excision |
| 10AA3836 | carcinoma | thickness, 4.6 mm | (30/11/2010) |
| Mela | Hypertension | SSM* grade 4; | Tumor excision |
| CHAN | | thickness, 2.5 mm | (23/7/2010) |
| 10AA4280 | | | |
| Lung CA | Cell line | Cell line | Cell line |
| H2030 | | | |

TRUS, transrectal ultrasound;
PSA, prostate-specific antigen;
PR, prostate;
MB, Manitoba;
MM, malignant melanoma;
SSM, superficial spreading melanoma.

### CTC Isolation by Filtration

Unlike most isolation techniques, the ScreenCell filtration device isolates the total CTC population, and not subpopulations, from 3 ml of patients' blood (Desitter et al., 2011). This isolation is done by size with the aid of a microporous membrane filter; therefore, expression levels and/or absence of cell surface antigens play no role in the separation. The 19-cm-long device consists of a filtration tank, a filter, and a detachable nozzle attached to it. This nozzle guides the insertion of a collection EDTA tube to it to gently vacuum suction the blood through the filter membrane leaving the CTCs on the membrane (Desitter et al., 2011). The 18-μm-thick polycarbonate membrane has circular pores (7.5±0.36 μm) that are randomly distributed throughout the filter (1×105 pores/cm2) (Desitter et al., 2011). The filtration process is quick (2-3 minutes), and it was determined herein that it preserves both the CTC morphology permitting assessment by 3D telomere assessment by for example Teloview and/or Teloscan and preserves microclusters/microemboli (which are also subjected to Teloview (FIG. 1.7, B-E). The filtration method was validated with spiked tumor cells; when two and five spiked cells per 1 ml of blood were used, the average number of cells recovered were 1.48 (SD, 0.71) and 4.56 (SD, 0.71), respectively (Desitter et al., 2011).

### Three-Dimensional Quantitative Fluorescence In Situ Hybridization

Quantitative fluorescence in situ hybridization was carried out on the nuclei of the CTCs captured by the filters according to the protocol earlier described in Louis et al., 2005 and Gadji et al., 2010. In brief, the cells on the filters are incubated in 3.7% formaldehyde/1× phosphate-buffered saline for 10 minutes followed by a 10-minute treatment with 50 μg/ml pepsin in 0.01 N HCl. The CTCs are postfixed to the filters with 3.7% formaldehyde/1× phosphate-buffered saline for 10 minutes before 8 μl of Cyanine 3 (Cy3)-labeled peptide nucleic acid probe purchased from DAKO (Glostrup, Denmark) is applied to them. The coverslipped and rubber cement-sealed filters on slides then undergo a 3-minute denaturation at 80° C. followed by a 2-hour hybridization at 30° C. The filters containing CTCs are washed twice 15 minutes each in 70% formamide/10 mM Tris (pH 7.4), subjected to a 5-minute wash in 0.1×SSC at 55° C., then washed twice 5 minutes each in 2×SSC/0.05% Tween 20. Finally, the nuclei are stained with 50 μl of 0.1 μg/ml 4′,6-diamindino-2 phenylindole (DAPI), dehydrated in graded concentrations of ethanol, and coverslipped with Vectashield® (Vector Laboratories, Burlington, Ontario) reagent ready for imaging.

### Three-Dimensional Image Acquisition

Images are acquired using a Zeiss AxioImager Z2 microscope (Carl Zeiss, Toronto, Ontario), equipped with AxioCam HR B&W camera and 63x/1.4 oil objective. The microscope is equipped with a Cy3 filter for detection of peptide nucleic acid probe-hybridized telomeres and a DAPI filter for nuclear DNA detection with AXIOVISION 4.8 software (Carl Zeiss). The Zeiss AxioImager Z2 was programmed to take 80 stacks of images at x and y=102 nm and z=200 nm to capture the different planes of the CTCs that are observed beside the pores or slightly in the pores. The same acquisition time was used to acquire Cy3 images of telomeres from each tumor type for quantitative comparison and analysis. The acquisition times used in milliseconds were given as follows: melanoma, 1290; colon cancer, 212; breast cancer, 212; prostate cancer, 546; lung cancer cell line, 173.6. Thirty interphase nuclei were imaged for analysis; deconvolution of the images was performed with a constrained iterative algorithm (Schaefer et al., 2001). The reconstructed 3D images were then exported as .tiff files into the TeloView™ program for analysis (Schaefer et al., 2001).

### TeloView™ Enabled 3D Image Analyses and Statistical Considerations

TeloView™ quantifies the telomere numbers, signal intensity, sizes, distribution, TAs, and nuclear volume for each sample. A graph of telomere numbers (y-axis) against signal intensity (x-axis) is plotted for each sample giving a first overview of the CTC 3D telomere profiles and of the presence/absence of subpopulations (FIG. 3 A-E). In addition, aggregate numbers and nuclear volumes are calculated and included in the analysis.

Statistical parameters considered for characterizing the CTCs in each patient sample into subpopulations are given as follows: 1) percentage of cells with aggregates (PCA), 2) average number of telomeres per cell (ANTC), 3) average number of aggregates per cell (ANAC), and 4) average nuclear volume (ANV).

Nested factorial analysis of variance was used to analyze the parameters above.

### Results

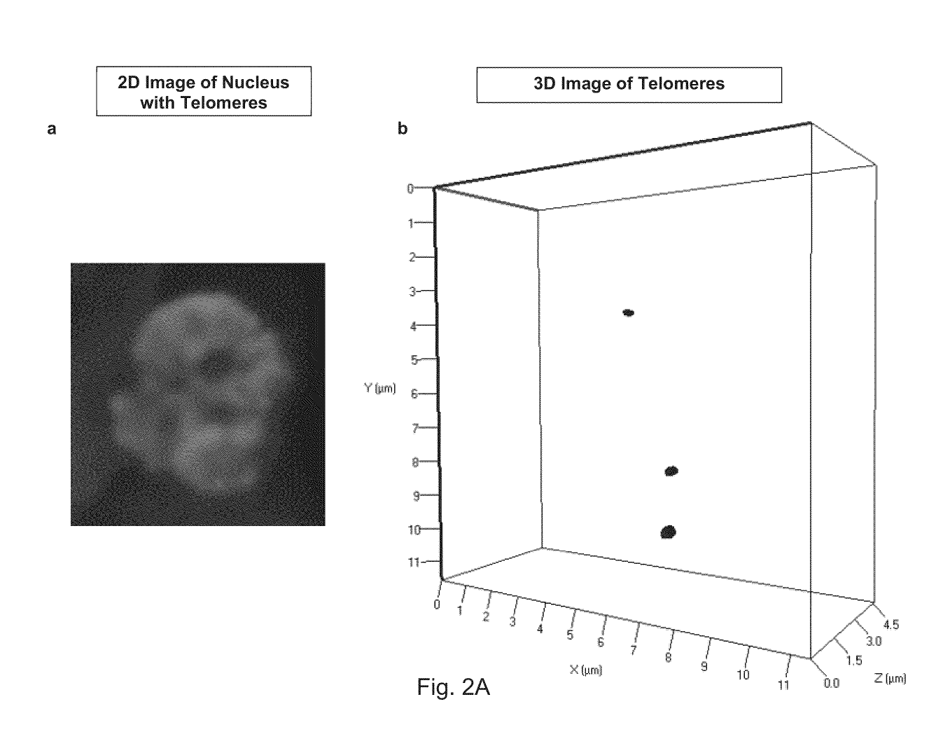

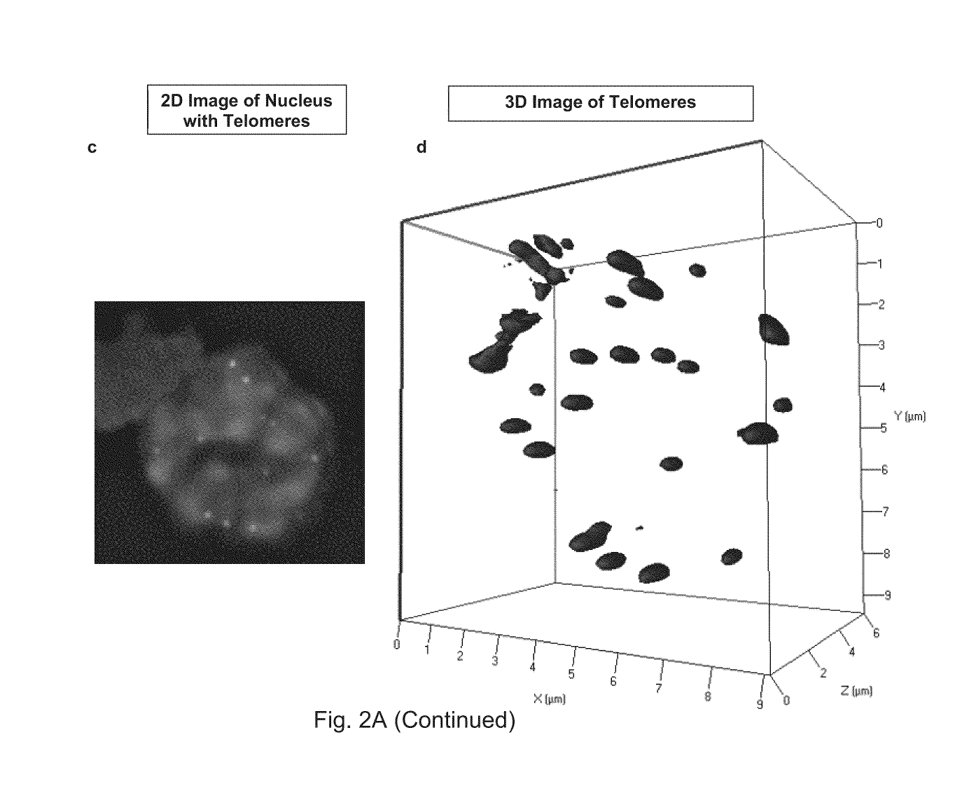

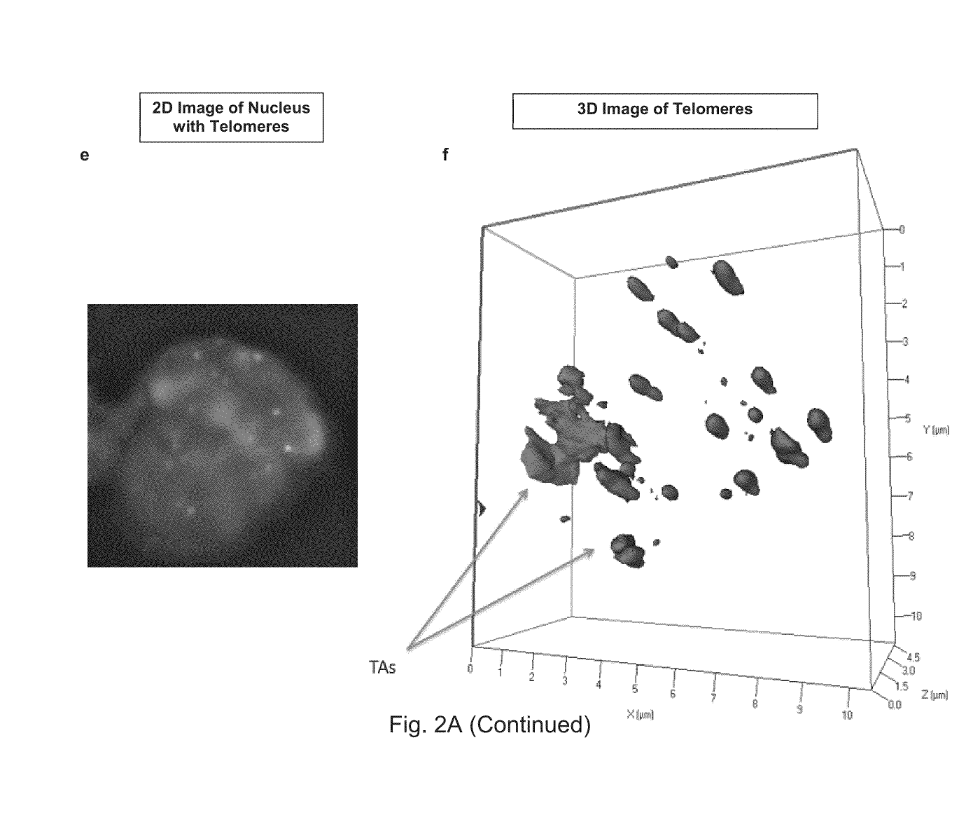

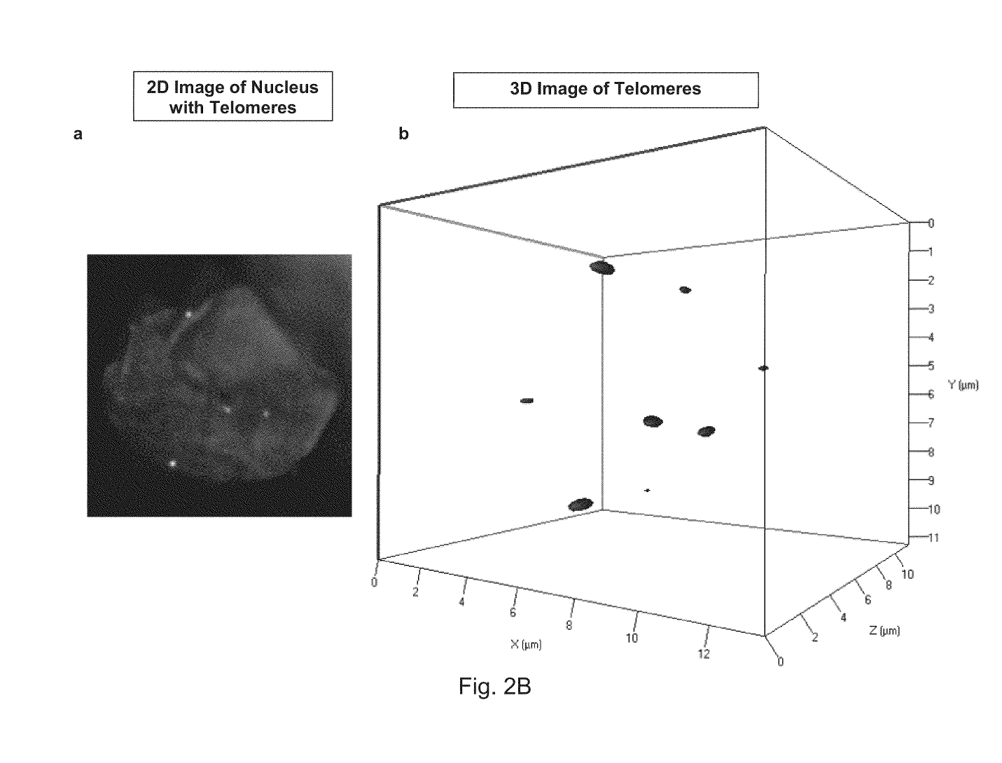

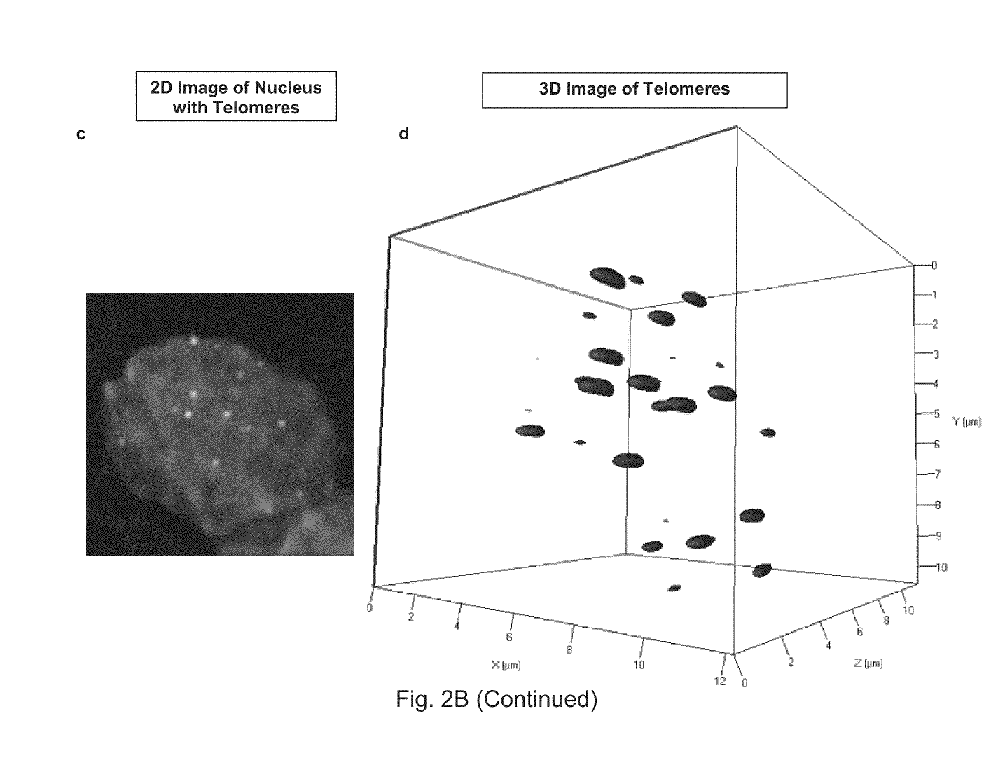

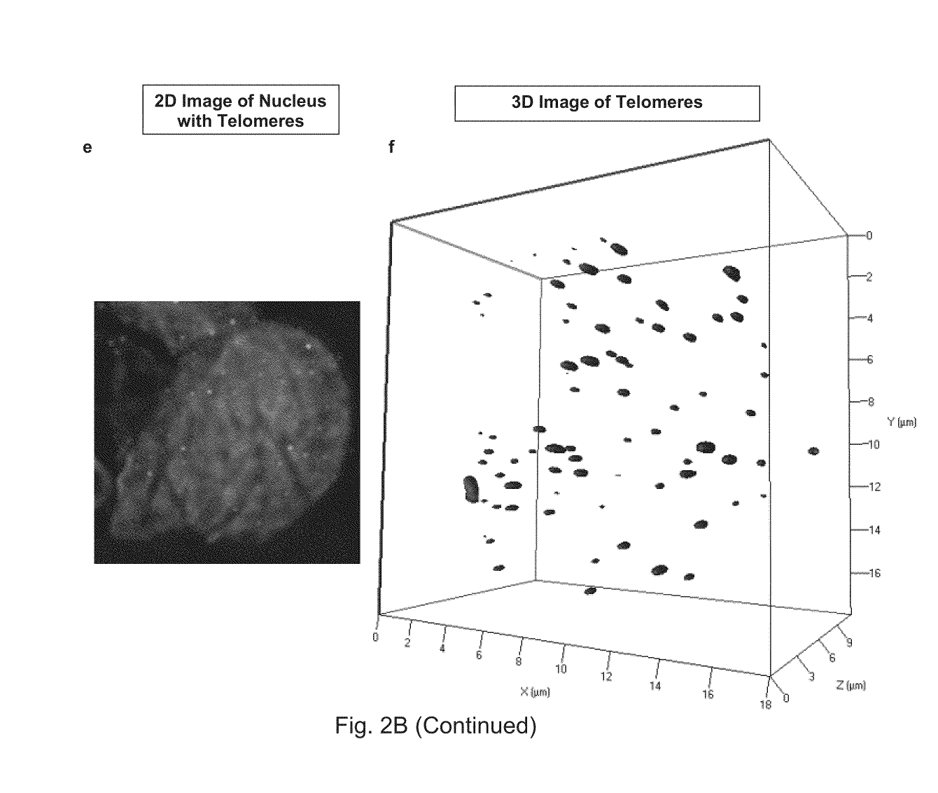

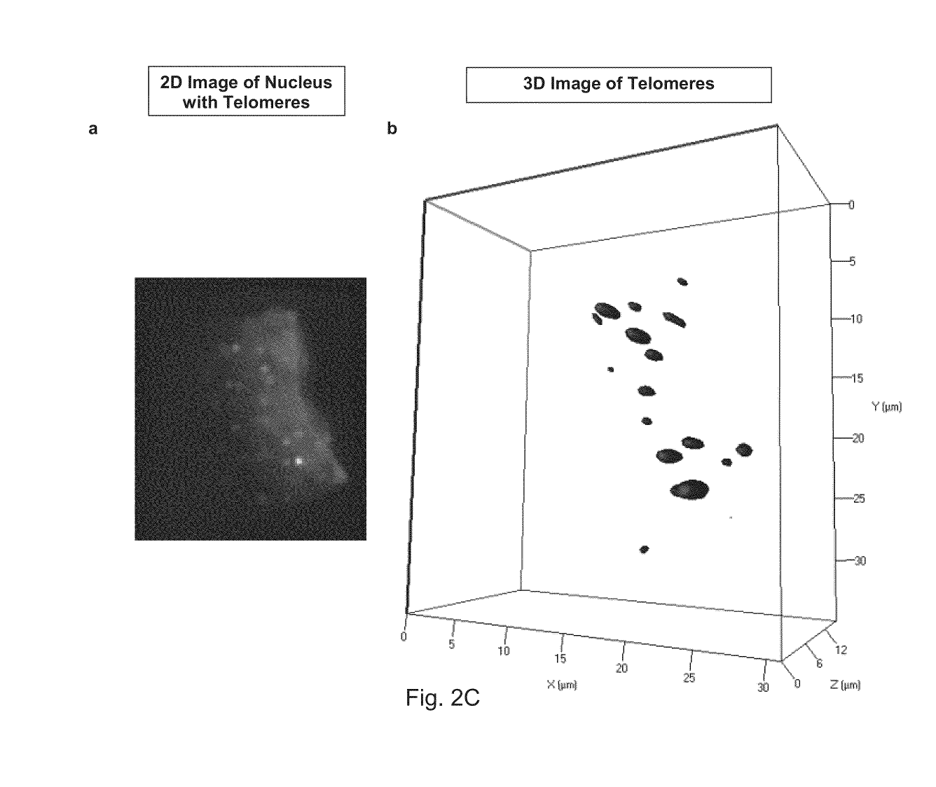

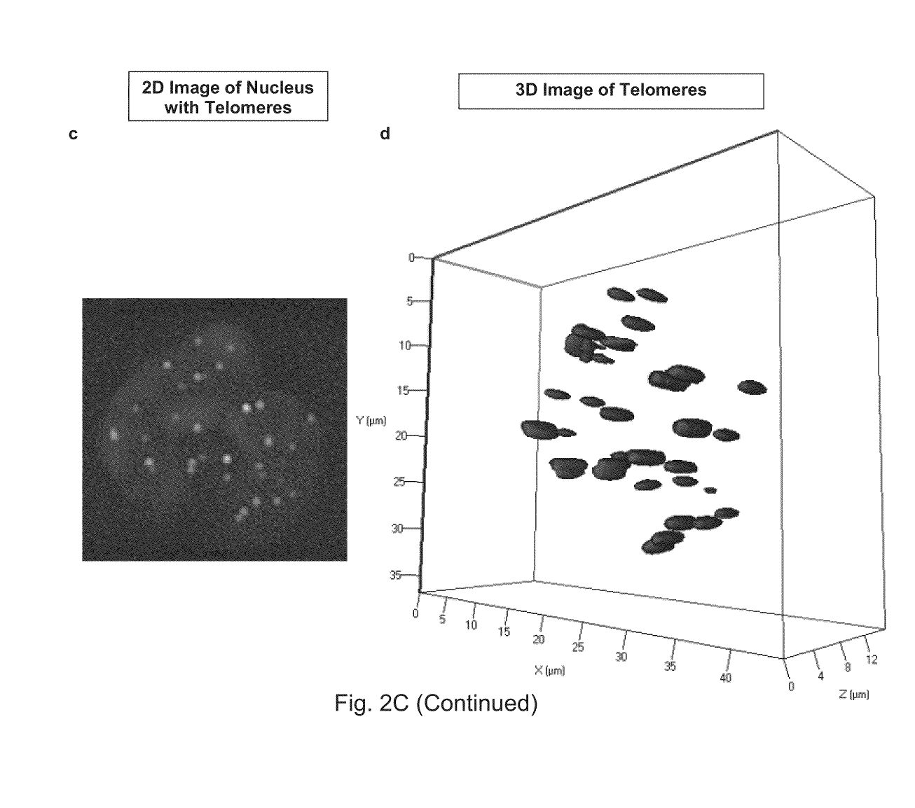

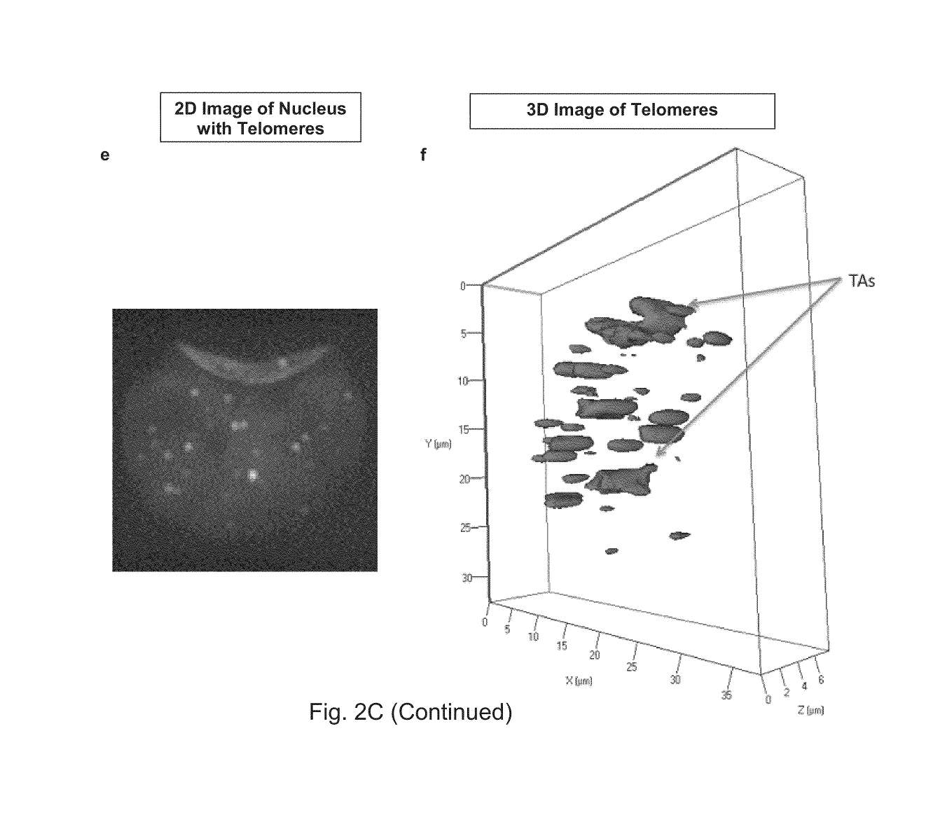

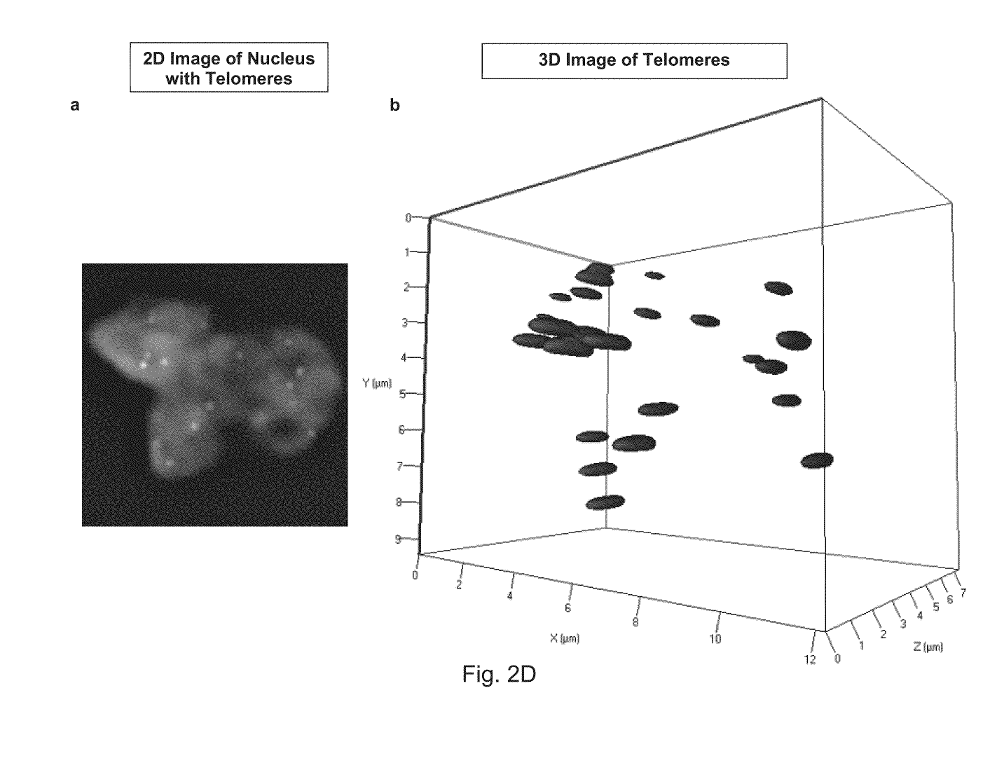

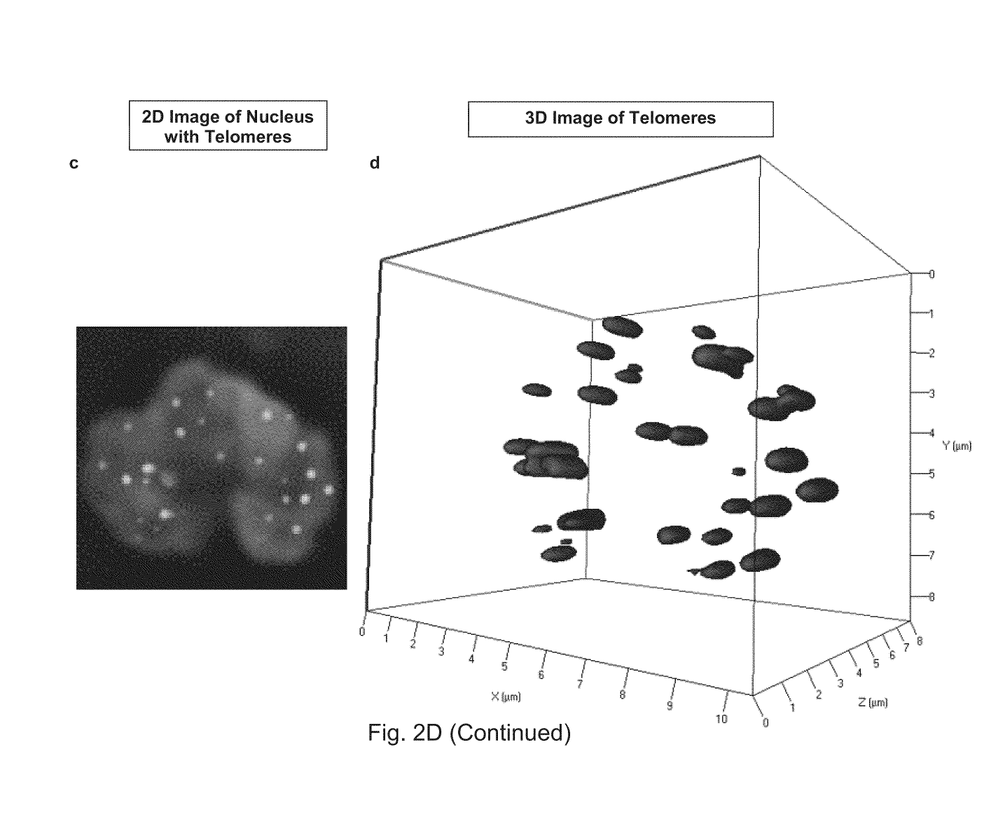

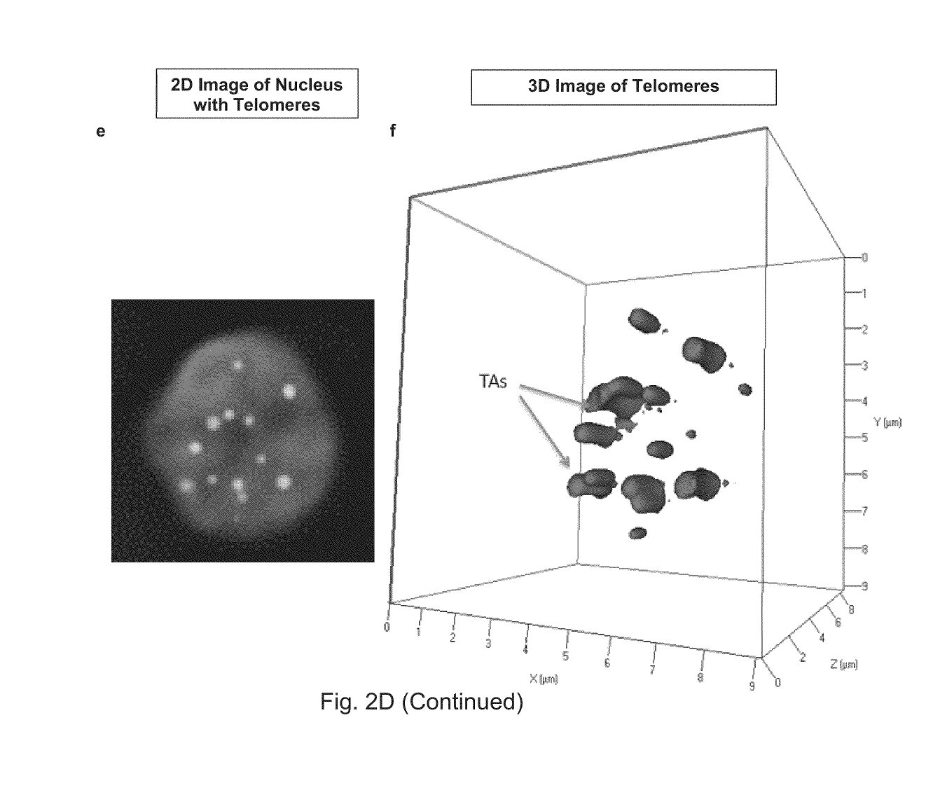

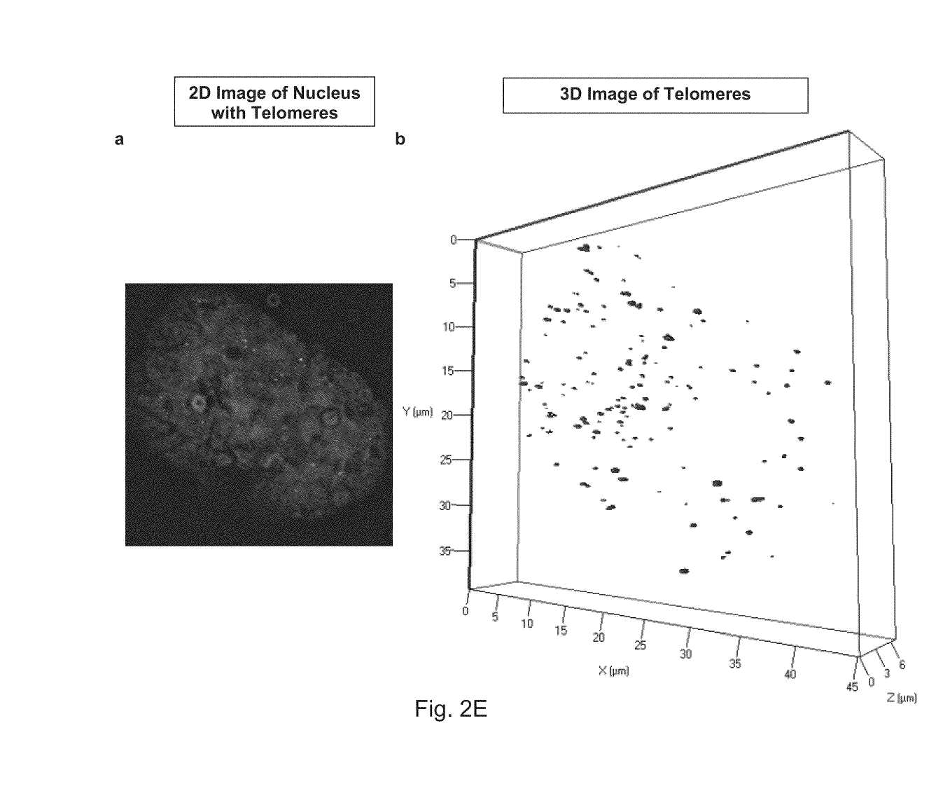

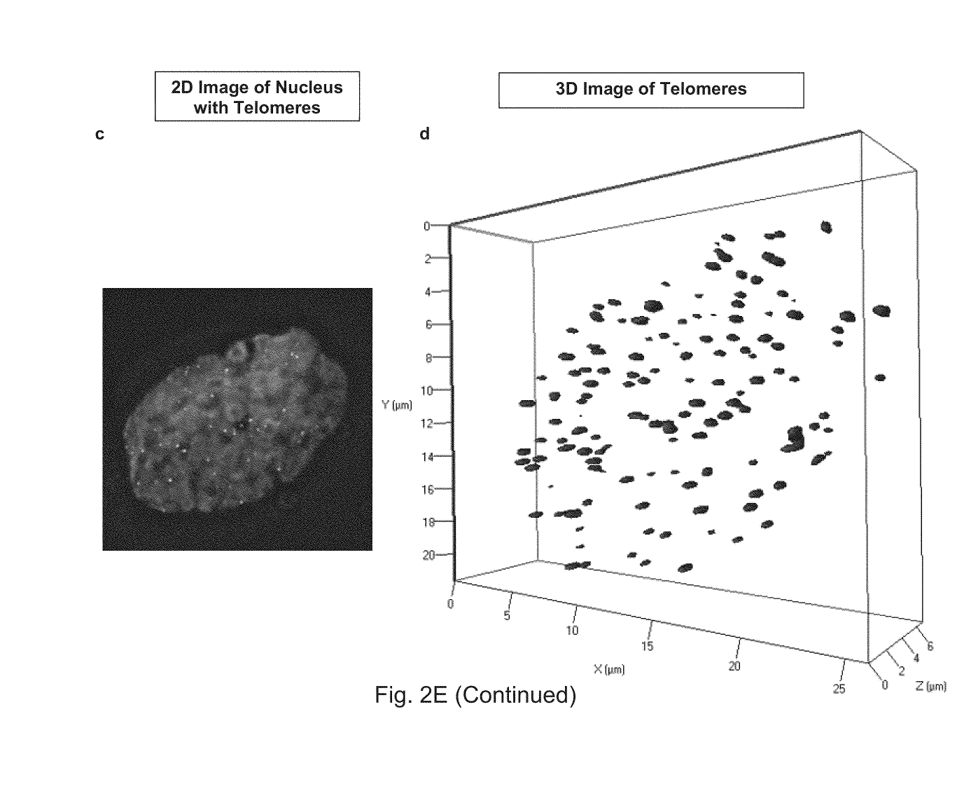

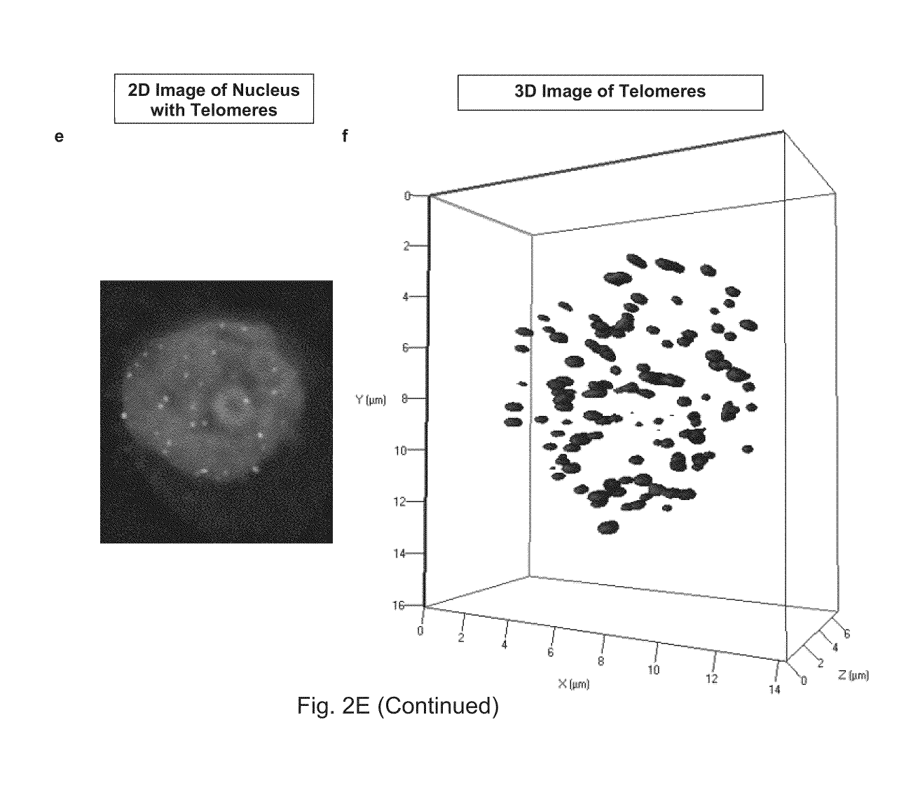

This study was designed to adequately characterize CTCs and potential subpopulations of CTCs in different cancer types using aberrations in the 3D architecture of telomeres due to telomere dysfunction as a common biomarker of chromosomal instability (CIN) and a potential surrogate of tumor aggressiveness. CTCs of prostate, colon, breast, melanoma, and nuclei of a cultured lung cancer cell line were analyzed and at least two distinguishable subpopulation patterns were seen in each patient sample in all of the tumor types (FIGS. 2 and 3).

TABLE 2
Summary of Data Obtained from 22 CTC Samples of Five Different Cancer Types
Analyzed and the Calculated Parameters Used in Characterization of the CTCs (PR,
prostate cancer; Colon, colon cancer; Br, breast cancer; Mela, melanoma; Lung CA, lung
cancer; PCA, percentage of cells with telomeric aggregates; ANTC, average number of
telomeres per cell; AVAC, average number of telomeric aggregates per cell; ANV, average
nuclear volume).

| | Percentage of | | | | | | | |
|---|---|---|---|---|---|---|---|---|
| | Telomere Intensity | | | | | | | Average |
| | Subpopulations in | | | | | | | Nuclear |
| | the Same Patient | | | | | | ANV | Diameter |
| CTC Sample | Low | Medium | High | PCA | ANTC | ANAC | (μm³) | (μm) |
| MB0181PR | 83.39 | 14.84 | 1.77 | 46.66 | 9.43 | 0.5 | 235.50 | 7.66 |
| MB0182PR | 94.58 | 0 | 5.42 | 37.04 | 8.88 | 0.48 | 294.54 | 8.25 |
| MB0189PR | 38.16 | 58.74 | 3.1 | 82.35 | 28.44 | 2.18 | 1443.89 | 14.02 |
| MB0211PR | 33.61 | 63.92 | 2.47 | 53.33 | 16.17 | 0.93 | 1154.02 | 13.01 |
| MB0213PR | 52.06 | 45.27 | 2.67 | 63.33 | 16.2 | 1.267 | 1062.18 | 12.66 |
| MB0216PR | 71.45 | 23.36 | 5.19 | 86.66 | 21.83 | 1.8 | 1032.66 | 12.54 |
| MB0217PR | 46.94 | 48.98 | 3.67 | 66.66 | 16.33 | 1.3 | 702.76 | 11.03 |
| MB0222PR | 32.17 | 65.22 | 2.61 | 86.66 | 23 | 2.367 | 657.97 | 10.79 |
| MB0239PR | 100 | 0 | 0 | 80 | 36.4 | 3.4 | 211.47 | 7.39 |
| Colon GUI 2F | 58.16 | 38.27 | 3.57 | 26.66 | 6.53 | 0.26 | 212.04 | 7.40 |
| Colon GUI 3F | 54.34 | 36.82 | 6.99 | 83.33 | 35.3 | 3.7 | 633.79 | 10.66 |
| Colon GUI 5F | 64.42 | 33.85 | 1.73 | 66.66 | 17.33 | 1.33 | 485.32 | 9.75 |
| BR MERT | 34.87 | 58.9 | 6.23 | 83.33 | 22.5 | 2.03 | 480.24 | 9.72 |
| 10AA5083 | | | | | | | | |
| BR MIC 10AA3956 | 77.36 | 20.22 | 2.42 | 70 | 24.73 | 1.73 | 469.73 | 9.64 |
| BR WUR 10AA | 54.98 | 39.58 | 2.44 | 75 | 19.19 | 1.66 | 413.92 | 9.25 |
| 2499 | | | | | | | | |
| BR MIC 10AA3934 | 35.38 | 62.01 | 2.61 | 73.33 | 17.9 | 1.66 | 564.07 | 10.25 |
| Mela GOD | 57.82 | 12.24 | 29.93 | 33.33 | 9.93 | 0.66 | 224.98 | 7.55 |
| 10AA4991 | | | | | | | | |
| Mela CAR | 85.52 | 37.17 | 3.36 | 36.66 | 13.9 | 0.46 | 292.49 | 8.24 |
| 10AA2213 | | | | | | | | |
| Mela SAU | 73.1 | 24.83 | 2.07 | 53.33 | 9.66 | 0.76 | 374.48 | 8.94 |
| 10AA2408 | | | | | | | | |
| Mela ROB | 76.64 | 21.77 | 1.13 | 63.33 | 14.7 | 1.1 | 866.57 | 11.83 |
| 10AA2521 | | | | | | | | |
| Mela GAU | 68.58 | 29.67 | 1.75 | 70 | 13.36 | 1.1 | 420.05 | 9.29 |
| 10AA3836 | | | | | | | | |
| Mela CHAN | 79.31 | 13.79 | 6.89 | 73.33 | 12.57 | 1.1 | 398.80 | 9.13 |
| 10AA4280 | | | | | | | | |
| Lung CA Cell Line | 66.32 | 32.22 | 1.46 | 100 | 104.057 | 12.81 | 1893.97 | 15.35 |
| H2030 | | | | | | | | |

The telomeres of five different tumor types were analyzed using TeloView™ which measures the number and size of telomeres and also identifies the presence of TAs (Vermolen et al, 2005; Knecht et al., 2009). Table 2 shows the different parameters computed by the TeloView™ program such as PCA, ANTC, ANAC, and ANV. With these data, the degree of telomere dysfunction can be assessed, thus giving insight into the level of CIN for each patient. The measurements offer the chance for earlier tumor detection and better cancer classification. Table 2 shows CTC nuclei of patient samples MB0189PR, MB0216PR, MB0222PR, COLON GUI3F, and BR MERT10AA5083PR and nuclei of H2030 lung cancer cell line with percentage of cells with TAs greater than 80%, patients MB0213PR, MB0217PR, COLON GUI5F, BR MIC10AA3956, BR WUR10AA2499, and BR MIC10AA3934 between 60% and 80%, and the remaining MB0211PR, MB0181PR, MB0182PR, COLONGUI2F, MelaGOD10AA4991, Mela CAR10AA2213, Mela SAU 10AA2408, Mela ROB10AA2521, Mela GAU10AA3836, and Mela CHAN10AA4280 less than 60%. Other important parameters that vary among the samples are the ANTC and the ANAC. Both the ANTC and ANAC have corresponding variations among the samples (Table 2). These data obtained from TeloView™ can be used to predict the complexity of genomic instability of the tumors. The TeloView™ analysis of CTCs of these five cancer types was done without prior knowledge of the patients' clinical data. The deductions and classifications resulting from the TeloView™ analysis was then compared with the clinical data obtained (Table 1).

### Morphology of the Filter Captured CTC Nuclei

The distinct 3D nuclear architecture of the CTCs was visualized through the DAPI filter before image acquisition. The captured CTCs were found either as solitary or clustered cells scattered around and sometimes slightly within the pores (FIG. 1.7). In animals, the importance of CTC clusters and tumor-lymphocyte mixed clusters as prognostic factors in metastasis process has been mentioned (Molnar et al, 2001; Glaves D., 1984). The CTCs are often irregularly shaped (FIGS. 1 and 2) and larger than other blood cells enabling their isolation due to their inability to pass through the filters' pores (7.5±0.36 μm) (Desitter et al, 2011); for example, the prostate cancer cell size ranges between 15 and 25 μm (Zheng et al, 2007). FIG. 1.7A shows hematoxylin and eosin (H&E)-stained prostate cancer CTCs (pointed out by arrows) captured by the filter device. These CTCs are clearly two and three times larger than the pores. The identities of the cells were confirmed by pathologic examination. CTCs sometimes display chromatin condensation, unlike most of the lymphocytes. Solitary lymphocytes mostly pass through the pores of the filter except in some instances where they are found in between pores. Lymphocytes sometimes also form lymphocyte-lymphocyte clusters or lymphocyte-CTC clusters that cannot go through the pores.

At ×40 microscope magnification, the density of the CTCs present can be appreciated in each sample with the presence of varying number of clusters noted. Both the density of the CTCs captured from 3 ml of each sample and the frequency of the clusters observed can give a preliminary insight to the status of the disease at the point when the sample was collected (Budd et al., 2006). FIG. 1.7 B-E, shows the isolation and preservation of CTC clusters in the filtered patients' blood.

At ×60 oil magnification, the varying sizes of the CTCs were observed with associated different chromatin condensation seen. Further analysis using TeloView™ measures the nuclear volume and this distinguishes CTCs from captured clumped lymphocytes that are smaller in size individually (Table 2). A switch to the Cy3 filter shows the hybridized telomere signals with varying signal intensity and numbers that give the first suggestion of different subpopulations within CTCs of the same patient's filtered blood.

### Telomere Numbers and TAs in CTCs

Cancer cells commonly exhibit an altered telomere number per cell nucleus with the telomeres often being shorter than those in normal cells. These alterations from the normal cell telomeres have been attributed to aneuploidy and genomic instability (Meeker et al., 2004).

Cy3-stained telomeres were analyzed and their signal intensities evaluated by TeloView™. The telomere signal intensity of a CTC nucleus is dependent on the number of TAs present in that CTC. This can be projected for the whole sample by calculating the PCA and the ANAC (Table 2). The program also calculates the ANTC in each nucleus. The variation in ANTC in the same sample may be an indication of the presence of CTC subpopulation and level of tumor aggressiveness (Mai et al., 2006; Gadji et al., 2010; Gadji et al., 2012). In FIGS. 2 to 3, different subpopulations of CTCs in the same patient are shown. The subpopulations of CTCs are identified on the basis of the differences in their telomere intensities, which can be due to varying number of telomeres, size of telomeres, or presence/absence of TAs. TAs are commonly seen in tumor cells (Mai et al., 2005; Mai et al, 2006) (FIGS. 2 Af, and Cf-Ef show prominent TAs) and their analyses has been shown to be useful in tumor characterization (Chuang et al., 2004; Mai et al., 2006).

FIG. 2 shows representative 2D and 3D images of isolated CTCs. The FIG. represents different subpopulations of CTCs present in the same prostate cancer patient (MB0181PR). In FIG. 2 the nuclear DNA is stained with DAPI (diffuse grey) and telomeres (bright dots), which are within the nuclei, are Cy3 stained. Images in FIG. 2 Aa and Ab, are of a cell that represents the subpopulation with low telomere intensity in this prostate cancer patient evident by the scanty number of signals observed. The two other CTC subpopulations represented in the same prostate cancer patient are the medium (FIGS. 2 Ac and Ad) and high (FIGS. 2 Ae and Af) telomere intensity CTCs. A similar classification is shown in the colon cancer patient GUI3F (FIG. 2B) with increasing numbers of telomeres seen along the classes of CTCs but a higher than normal number of telomeres observed in CTCs that belong to the high intensity subpopulation (FIGS. 2, Be and Bf). This irregularly high number of telomeres seen is one of the features of cancer cells that result from CIN (Mai et al, 2006). FIG. 2C shows 2D and 3D images of cells representing subpopulations in a breast cancer patient: BR MIC10AA3934, with low (FIGS. 2 Ca and Cb), medium (FIGS. 2 Cc and Cd), and high (FIGS. 2 Ce and Cf) intensity telomere signals. FIG. 2D shows subpopulations in a melanoma patient: Melanoma CAR10AA2213, which is similar to subpopulations in breast cancer patient (BR MIC10AA3934) (FIG. 2C) except for the presence of more TAs (FIG. 2Df) in the melanoma patient. The lung cancer subpopulations in cell line H2030 are shown in FIG. 2E. It was noted that this lung cancer cell line generally has high telomere numbers, but their telomere sizes vary. The telomere size was used to group the CTCs into subpopulations (FIG. 2E).

From the results shown in FIG. 2 it is clear that different subpopulations of CTCs are present in the same cancer patients and that these subpopulations can be identified by TeloView™ analysis. In the same tumor type, the variations in telomere intensities in addition to the presence and frequency of TAs can with larger patient cohorts, permit the classification of cancer into stages of progression and aggressiveness with the prospects of improving cancer management.

Telomere Numbers versus Telomere Intensity Measured in CTCs

The TeloView™ program (Vermolen et al., 2005; Knecht et al., 2011) plots a graph of telomere length (signal intensity) on the x-axis against the number of telomeres on the y-axis. The signals with the same intensity fall on the same spot on the graph and this gives a picture of the distribution of CTC subpopulations within each patient's filtered blood. For normal cells, this plot usually has a single peak, which ranges between 40 and 60 telomeres per nucleus on the y-axis (de Vos et al., 2005). A direct comparison of prostate cancer CTCs and lymphocytes from the same patient (MB0239PR) is shown in FIG. 4. Not only are the numbers of telomeres detected different between CTCs and lymphocytes of the same patient but also did we measure a size difference between the two cell types with average sizes of CTC nuclei being more than three-fold larger than those of the lymphocytes of the same patient, captured on the same filter (FIG. 4).

CTCs of all patients give 3D telomeric profiles with either a very high number or very low number of telomeres (FIG. 3, A-E). FIG. 3 shows plots of the different representative CTCs' telomere numbers against their intensities. Many deviations from the 3D telomere profiles of normal cells (Chuang et al., 2004; de Vos et al., 2009; Vermolen et al., 2005) (FIG. 4) were observed; the striking regular finding in the graphs from these CTCs is the presence of multiple peaks, which represent different subpopulations in the samples. The plots revealed the different subpopulations of CTCs in the same patient shown by the vertical line demarcations in the graphs. The subpopulations can be identified according to their signal intensities, i.e., low, medium, and high intensities (FIG. 3, A-E). FIG. 3A is a plot of a prostate patient's signal intensity against number of telomeres, the telomere numbers peak at 18 (below the normal range (Chuang et al., 2004; de Vos et al., 2009; Vermolen et al., 2005)), and there are three populations identified by line demarcations on the plot, i.e., low, medium, and high intensity groups (FIG. 3 A-E). The plot for GUI3F (Colon CA) in FIG. 3B is a sharp contrast to FIG. 3A with telomere numbers having a peak at 110 (FIG. 3B). There is disparity in different cancer types that can be studied further with larger cohorts of the same cancer type. Although the plot for BR MIC10AA3934 (FIG. 3C) has a telomere number peak of 45, it has multiple peaks that signify the different subpopulations present in the same patient (FIG. 3C). The zigzag nature of the plot for melanoma gives it a peculiar pattern (FIG. 3D); it also has its highest telomere number peak at 19 (below the normal range). Three distinct subpopulations of telomere intensities can be identified in this plot (FIG. 3D). The plot for lung cancer cell line depicts its high telomere number with a peak of telomere numbers at 290, most of which are short telomeres as earlier shown in 3D images (FIG. 2D, images b and d).

### Telomere Structural Changes in Cancer Types

When analyzing five different cancer types, it was observed that there might be a characteristic feature in the 3D telomere architectural changes seen in each of these cancer types. Although they all exhibit the presence of subpopulations of CTCs in the same patient sample, the telomeres of these cancer cells may tend to have similar features in each tumor type. The prostate, melanoma, and breast cancer CTCs may tend to form high numbers of TAs (FIGS. 2 Af, Df and Cf). The colon cancer CTCs and lung cancer cell line may tend to have significantly increased numbers of telomeres (FIGS. 2 Bf and Ef). The peculiarities seen in telomeres of different tumor types are also exhibited in the plots of their telomere numbers against telomere intensities (FIG. 3 A-E). The architectural alterations seen in the telomeres seem to be specific to each cancer type.

### Frequencies of CTC Subpopulations

Three milliliters of blood per patient captures CTCs present in this volume of blood and allows for the detection of those CTCs that are present in that sample. Duplicate and triplicate samples taken at the same time and from the same patient will result in the isolation of varying numbers of CTCs due to their rare presence per milliliter blood. This will also impact on the frequencies of individual subpopulations detected. We can therefore only conclude that CTC subpopulations are present, but the frequency of each population may vary in small sample volumes. Two example are provided here to illustrate this point. Patient GUI with colon cancer had three 3-ml blood samples examined (2F, 3F, and 5F) and their 3D nuclear architecture analyzed. 2F, 3F, and 5F have telomeres with low intensity at 58.16%, 54.34%, and 64.42%, respectively; medium intensity telomeres were found at 38.27%, 36.82%, and 33.85%, respectively, and high intensity telomeres had frequencies of 3.57%, 6.99%, and 1.73%, respectively (Table 2). This is a fairly consistent representation of the telomere intensity subpopulations in three different samples of the patient GUI.

A different example is represented by the breast cancer patient MIC. Two different samples (10AA3956 and 10AA3934) were obtained at the same time from the patient, and the 3D nuclear analysis of the CTCs revealed the presence of three different telomere subpopulations. 10AA3956 and 10AA3934 had percentages of telomeres with low intensity of 77.36 and 35.38, medium intensity of 20.22 and 62.01, and then high intensity of 2.42 and 2.61, respectively (Table 2). The frequency of each population is more variable (except in the high telomere intensity group) than it was in patient GUI. Therefore, the average of multiple samples obtained at the same time will only provide confirmation of the presence of distinct CTC subpopulations in a patient but will not give an absolute distribution frequency.

The numbers of CTCs isolated are predictive and can be quantitated, but as the sample size is small, the numbers are not absolute numbers of CTCs for a patient.

### Frequency of Circulating Tumor Microemboli

The presence and frequency of circulating tumor microemboli in circulation as shown in FIG. 1.7 B-E, H&E-stained filtered melanoma blood, can be estimated using the combination of CTC isolation by filtration and 3D analysis of CTCs. These circulating tumor microemboli can lead to clogging of small blood vessels, thus causing anemia in the region supplied by the affected vessels (Kane et al., 1975). This can result in increased morbidity and could be included as a prognosticator that could enhance the classification and management of cancer patients.

### Example 3

### Isolation and Characterization of CTC Cells from Patients with Prostate Cancer

CTC cells were isolated from patients with prostate cancer as described for Example 1&2. The 3D nuclear organization of the telomeres within the nuclei of the isolated cells was also analyzed as described for Example 1&2.

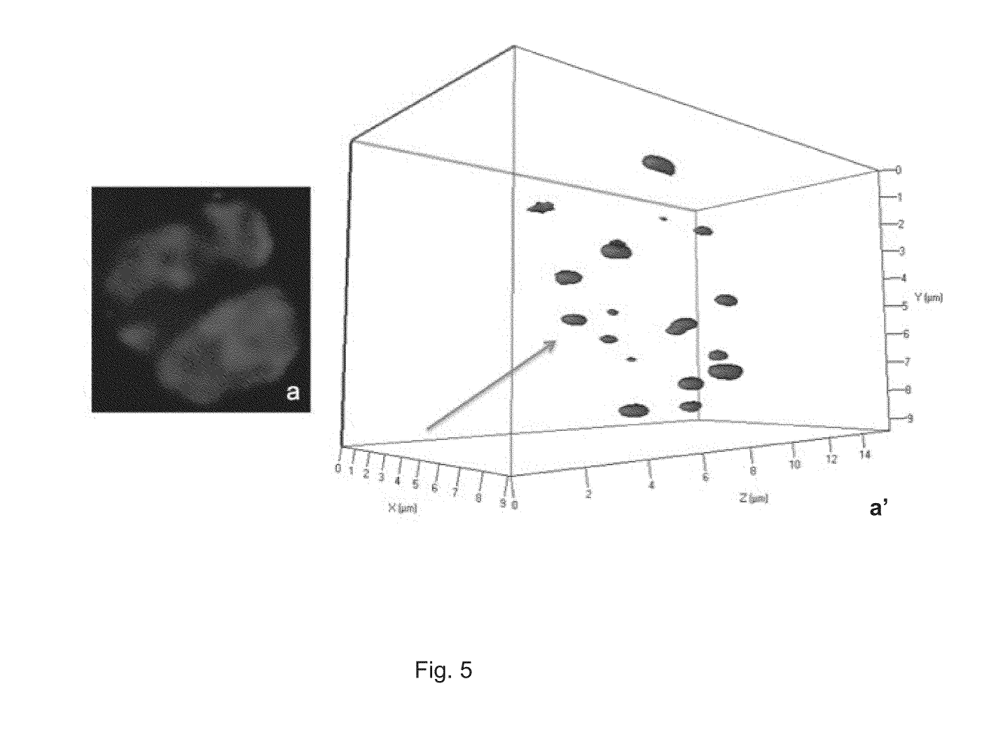

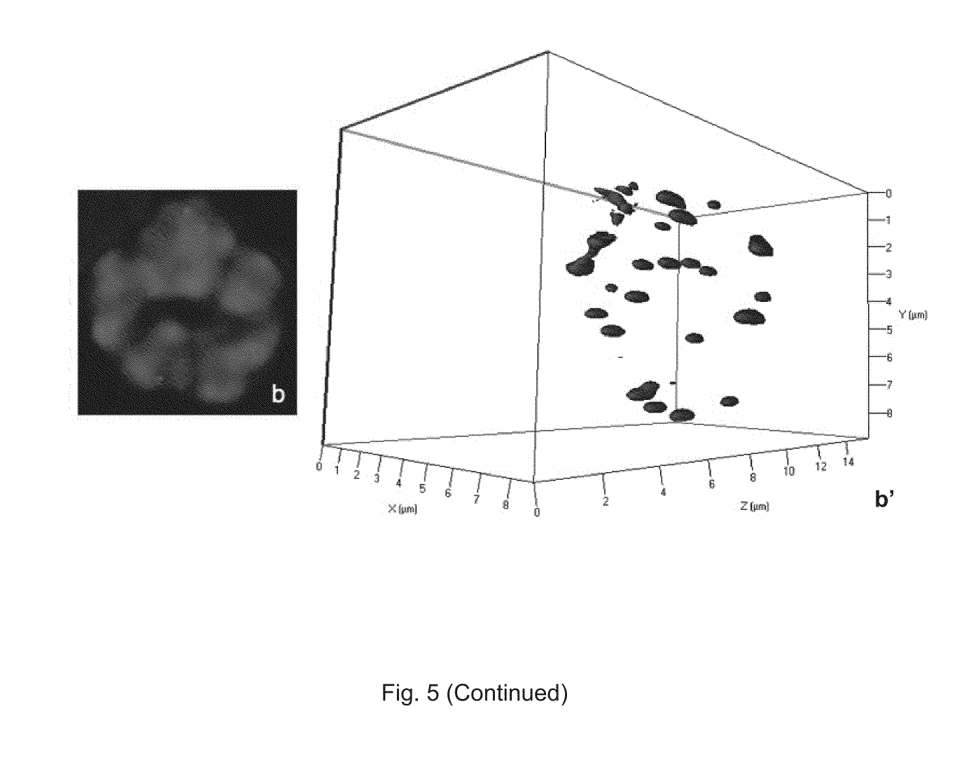

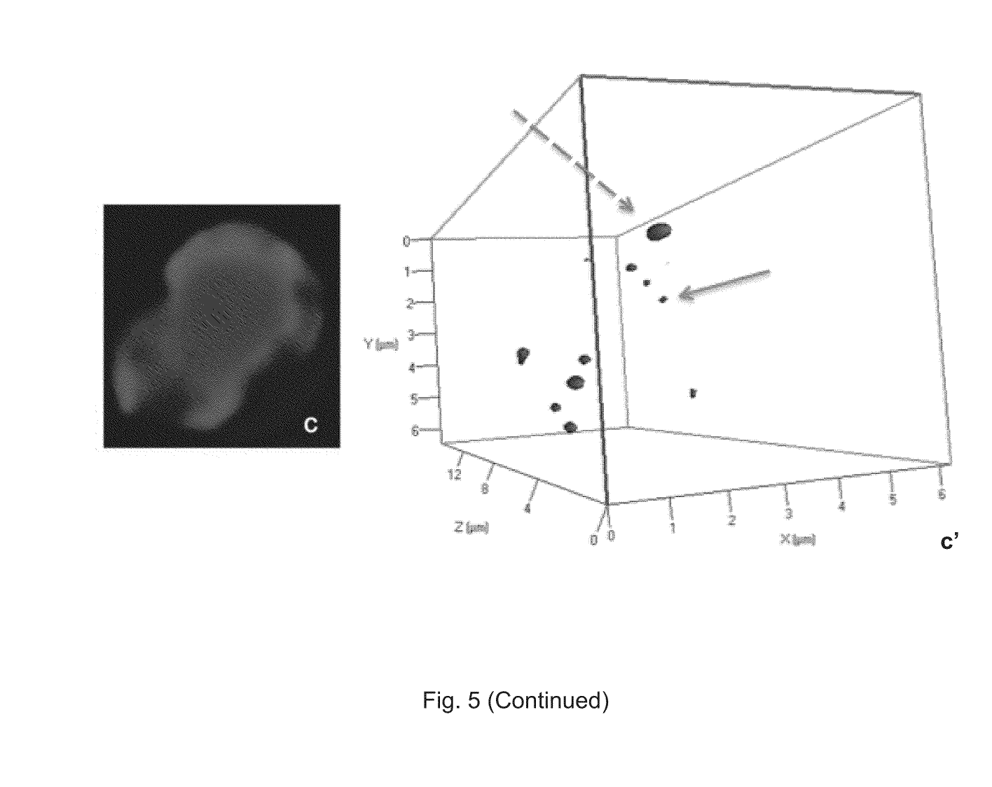

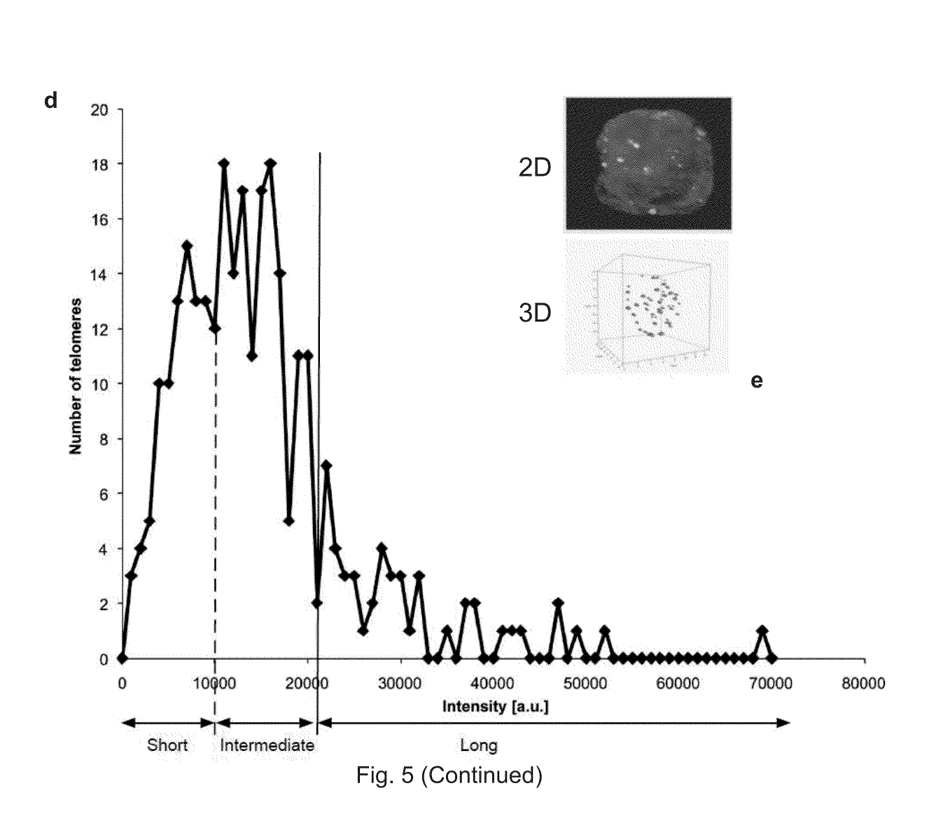

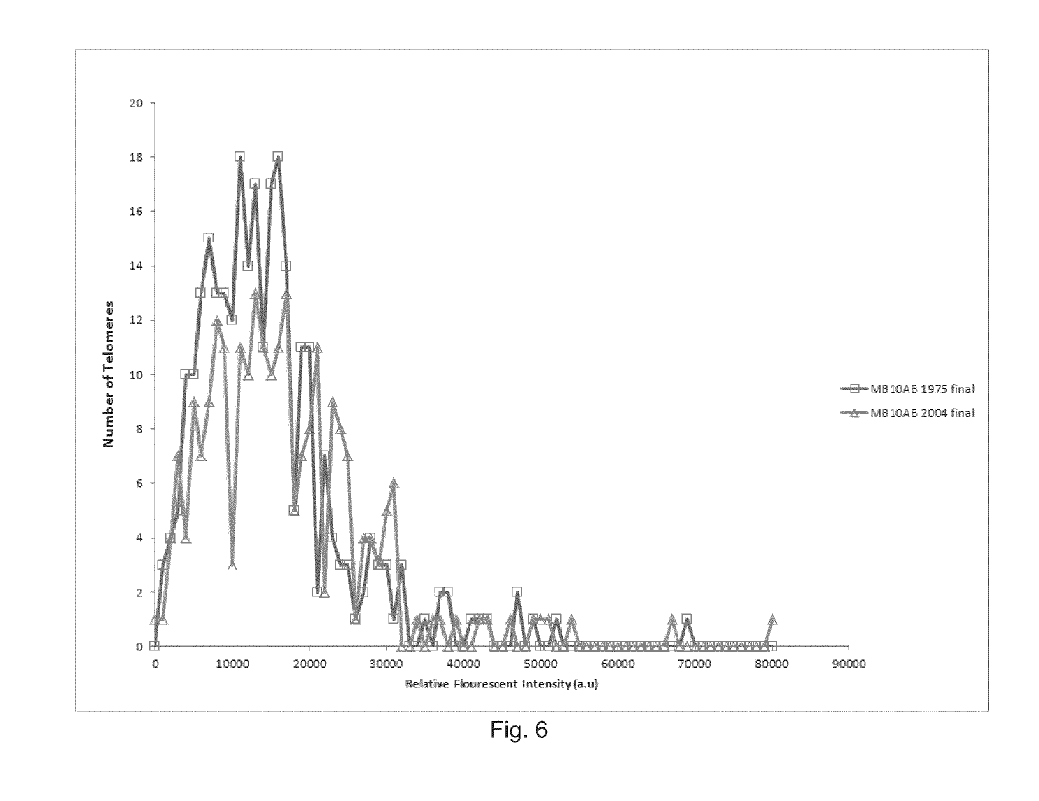

FIG. 5 shows the results of 2D and 3D telomere analysis of cells from patient sample MB 10A 1975. MB 10A 1975 has metastatic high grade prostate cancer. FIG. 6 shows a comparison between the telomere analysis of sample MB 10A 1975 and patient sample MB 10A 2004. MB 10A 2004 intermediate risk localized prostate cancer.

The numbers of CTCs were higher in MB 10A 1975 (>40/3.5 ml of blood) than MB 10A 2004 (30/3.5 ml blood). As show in FIG. 6, three sub-populations were found in the CTCs from MB 10A 1975 based on intensities alone (0-10000; 10001-20000; 20001 to 80000). Two sub-populations were found in the CTCs from MB 10A 2004 (0-30000 and 30001-80000). The complexity of telomere dysfunction was greater in MB 10A 1975.37% of CTCs have aggregates in MB 10A 2004 while the number is 46% in MB 10A 1975.

### Example 4

### Isolation and Characterization of CTC Cells from Intermediate Risk Prostate Cancer Patients

CTC cells were isolated from patients with prostate cancer as described for Example 1&2. The 3D nuclear organization of the telomeres within the nuclei of the isolated cells was also analyzed as described for Example 1&2.

The study group consisted of 200 patients over 5 years. 196 samples were collected. All patients were determined to have CTCs, and were determined to fall within three groups based on their 3D telomeric profiles.

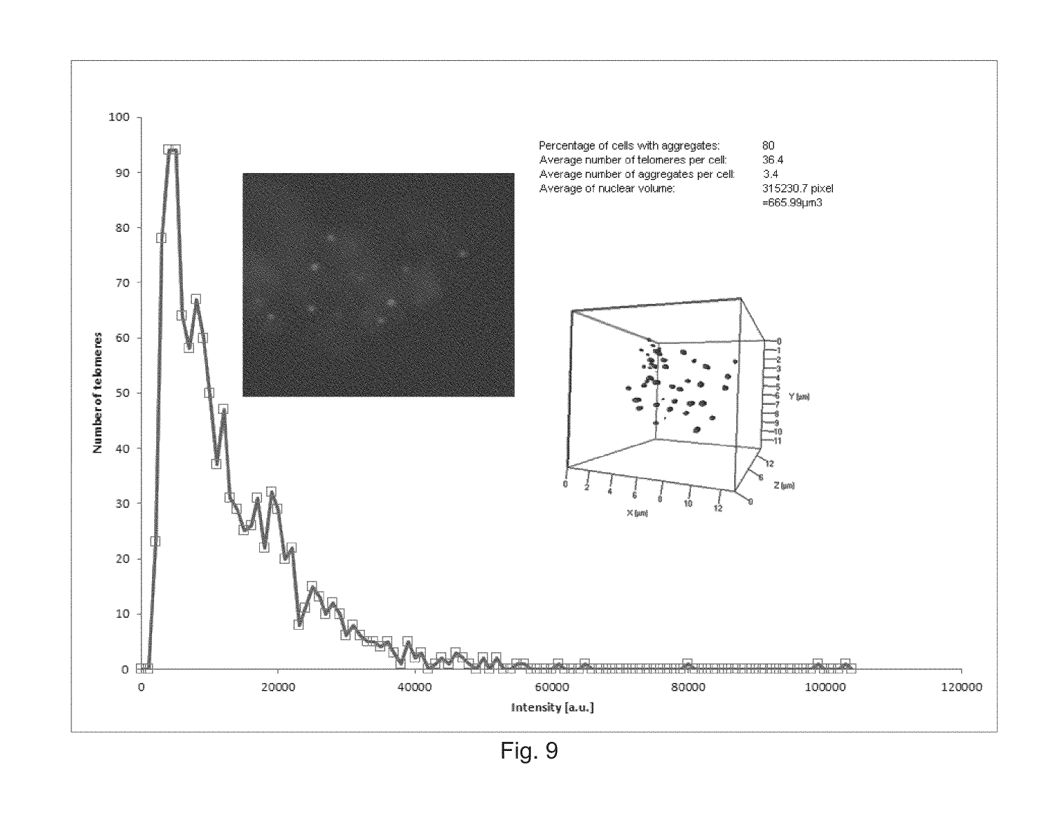

Group I patients showed one major population of CTCs with relatively short telomeres and a high level of telomeric aggregates. The CTCs also showed altered number of telomeres per nucleus. The profile of a Group I CTC is shown in FIG. 9.

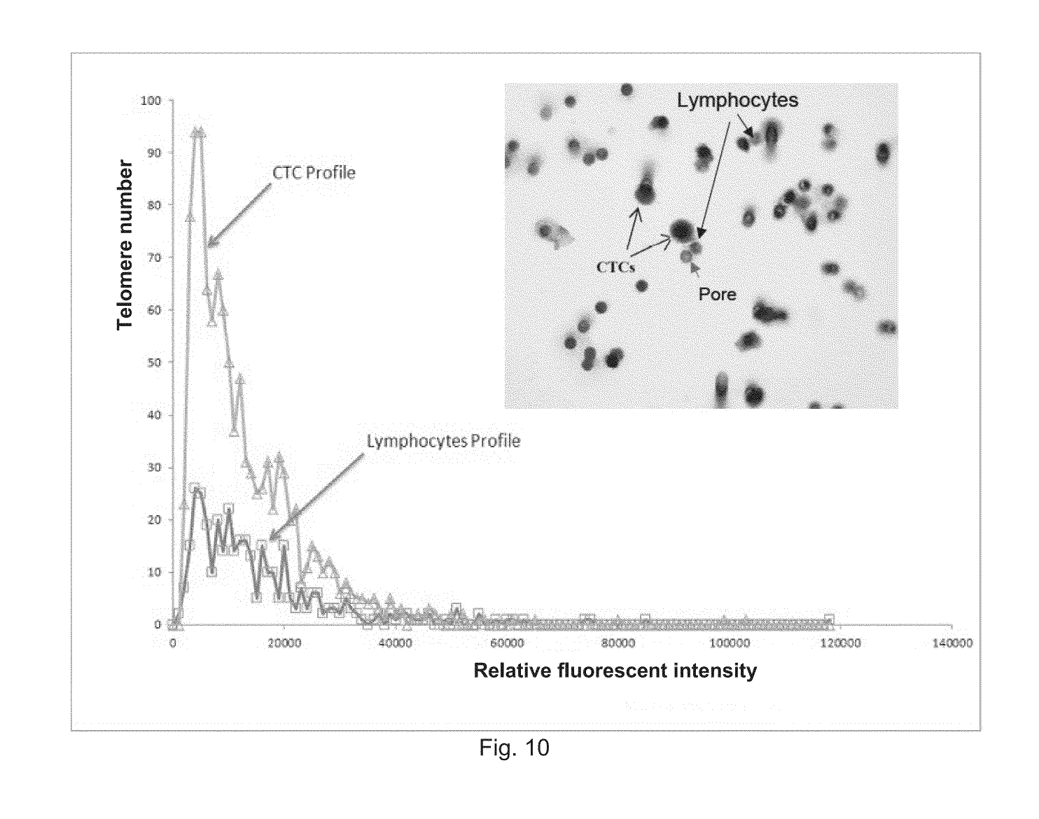

The 3D telomere profile of CTCs was compared to the 3D telomere profile of normal nuclei, as for example from lymphocyte cells. Normal nuclei show 40-60 telomeres at 200 nm optical resolution, do not show telomeric aggregates, have small, intermediate, and long telomeres in each cell, where the length of the telomere is age dependent. In contrast CTCs show increased or decreased numbers of telomeres per nucleus, enlarged nuclear volumes, telomeric aggregates and patient-specific telomere intensities; a patient may have one or more populations of CTC with the above characteristics and variability is possible dependent on the patient and stage of disease. A comparison of the a CTC telomere profile compared to a lymphocyte profile is shown in FIG. 10.

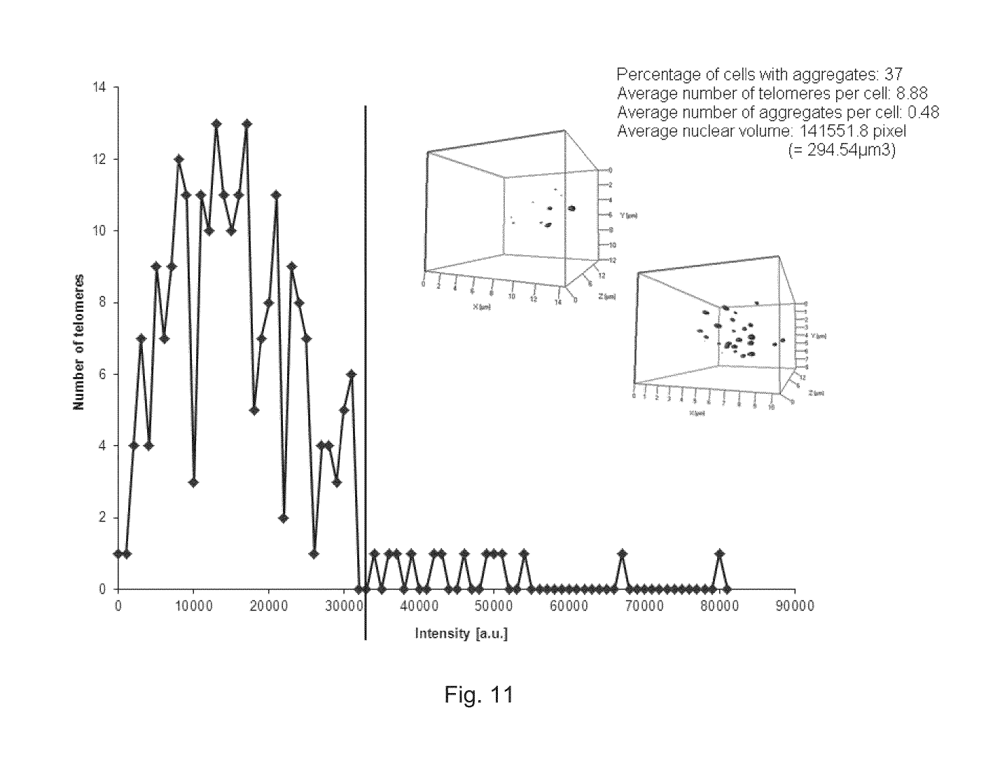

Group II patients showed two populations of CTCs, and the CTCs had a low number of telomeres and intermediate level of aggregates. A sample profile of a Group II patient CTC is shown in FIG. 11.

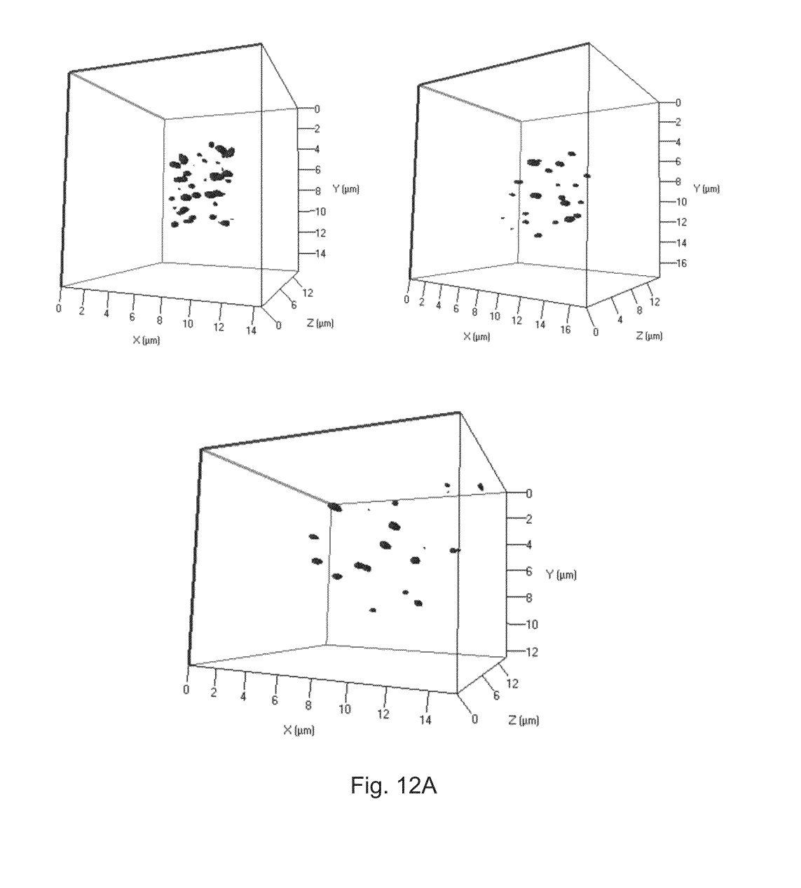

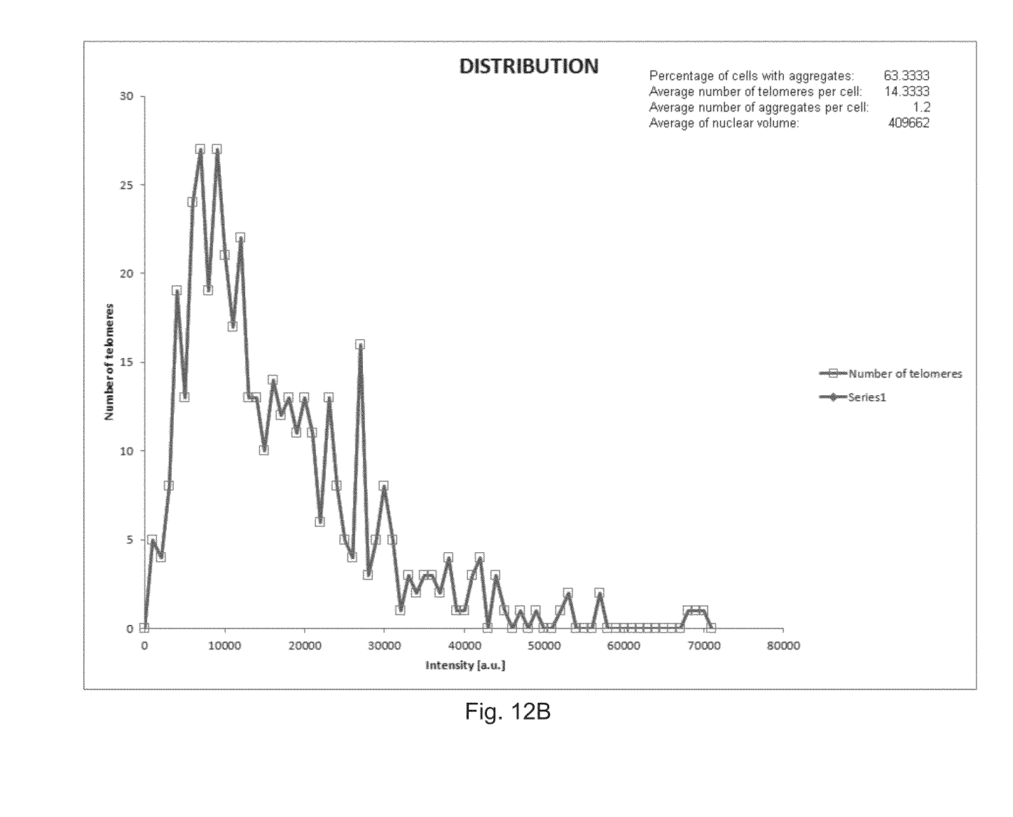

Group III patients showed three populations of CTCs, the CTCs having low to intermediate levels of telomeres/nucleus, and a heterogeneous level of aggregates, from intermediate to high. FIG. 12A depicts a 3D telomere FISH visualization of the telomeres of CTC populations from Group III patients. A sample profile of a Group III patient CTC is shown in FIG. 12B.

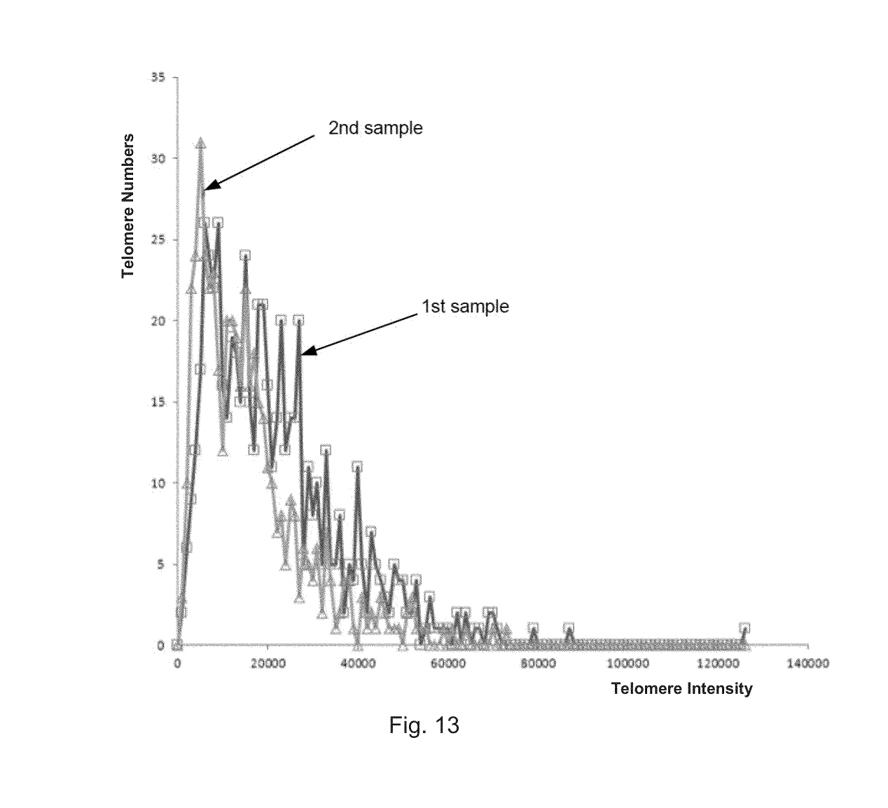

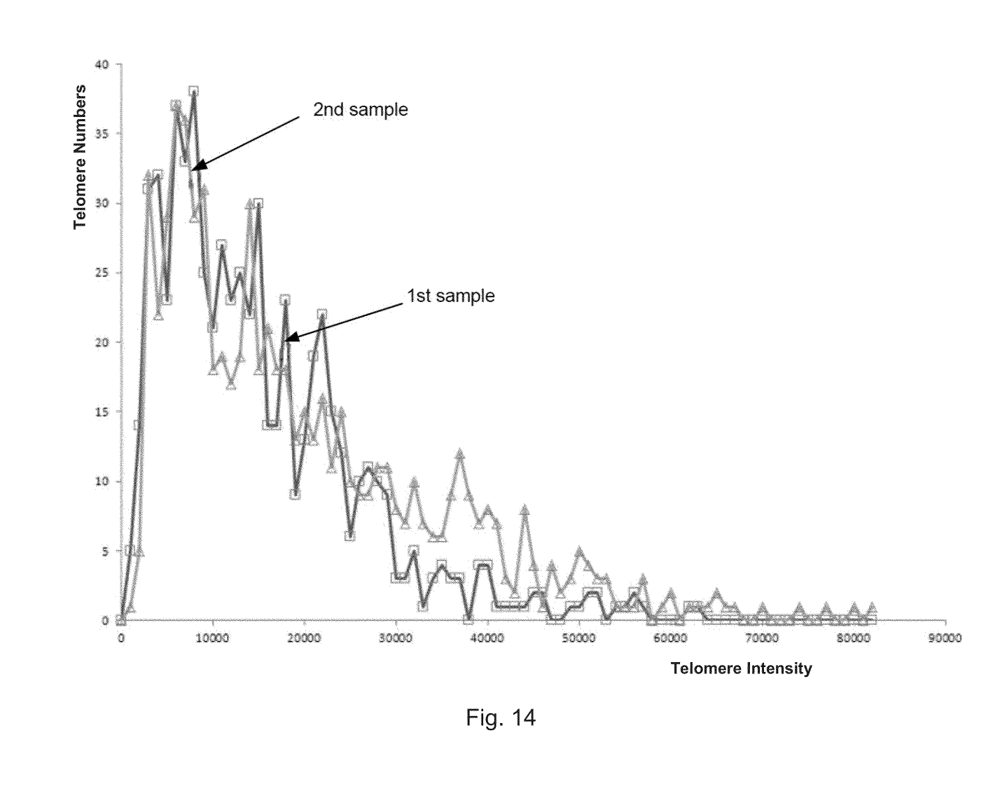

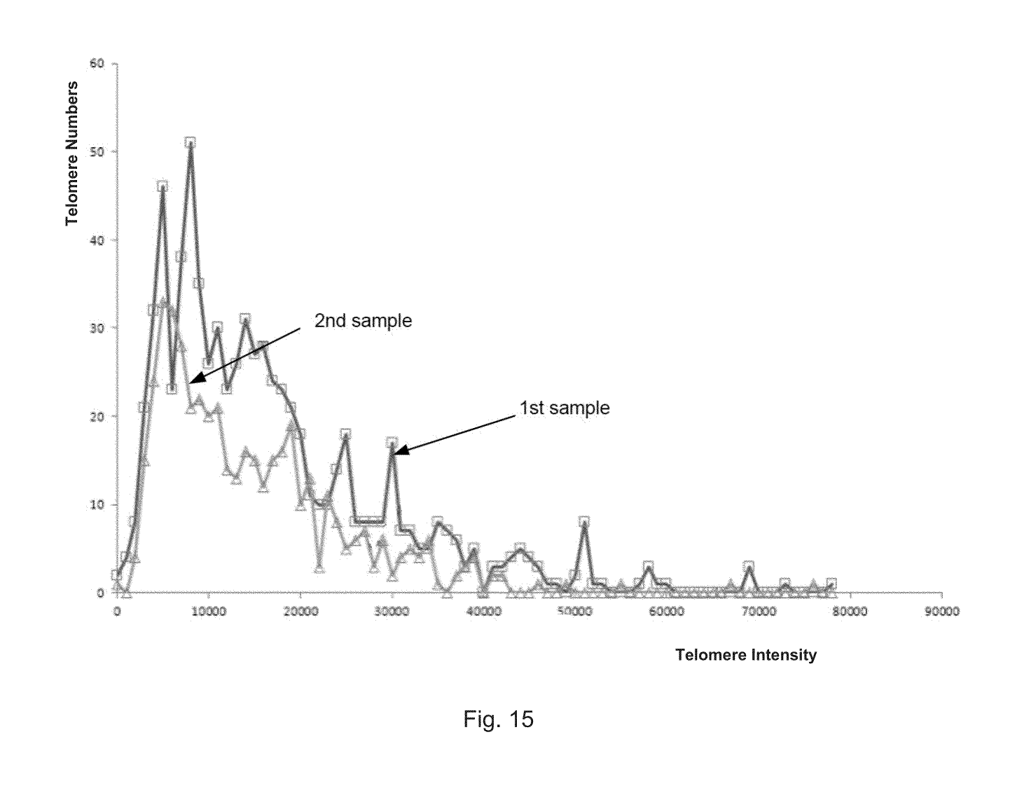

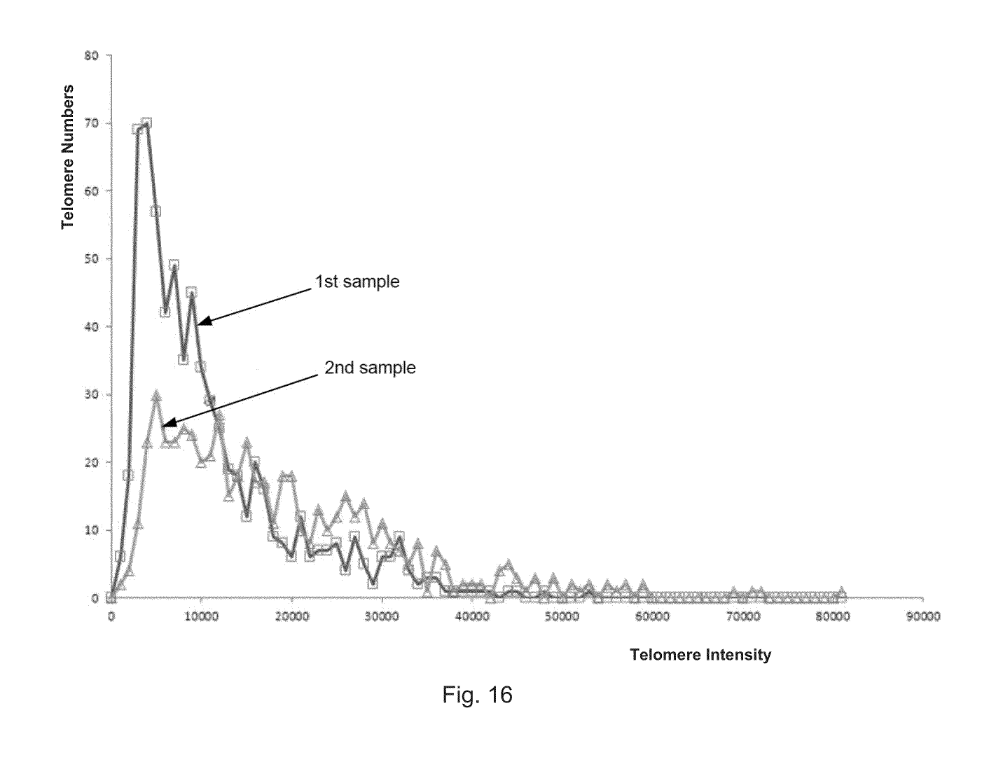

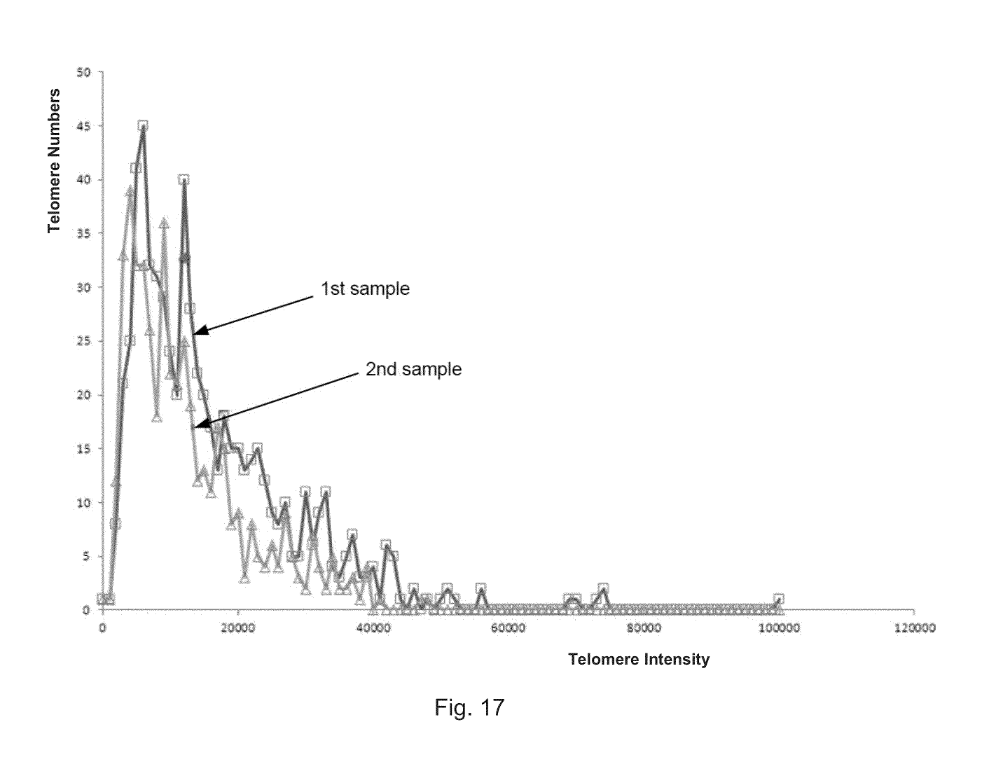

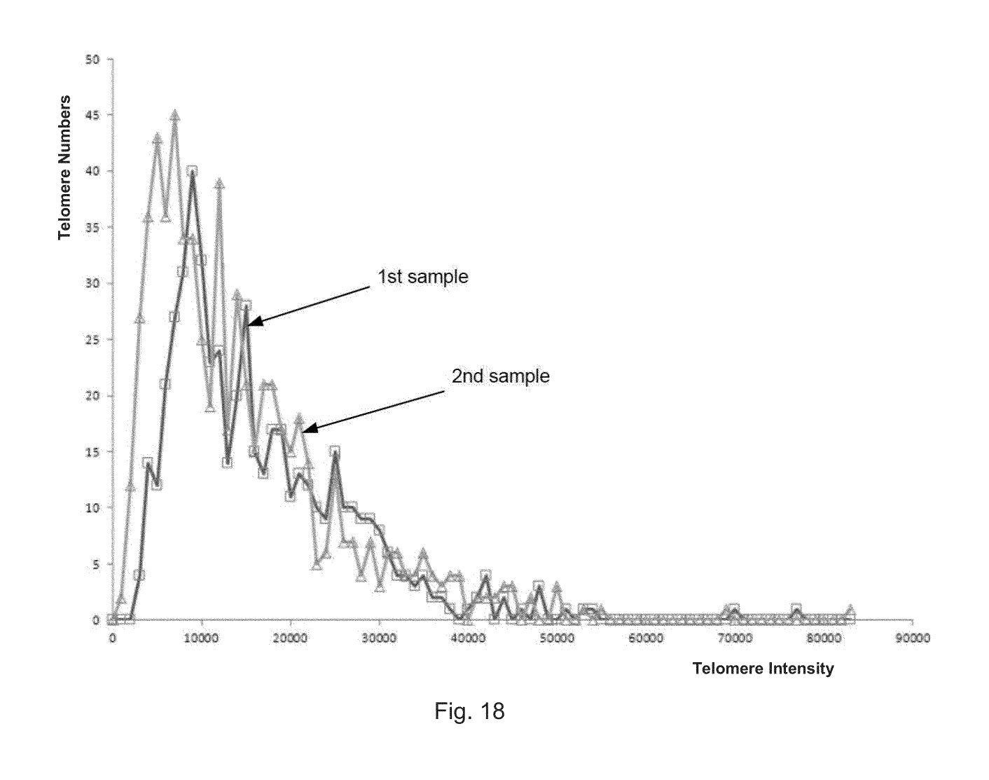

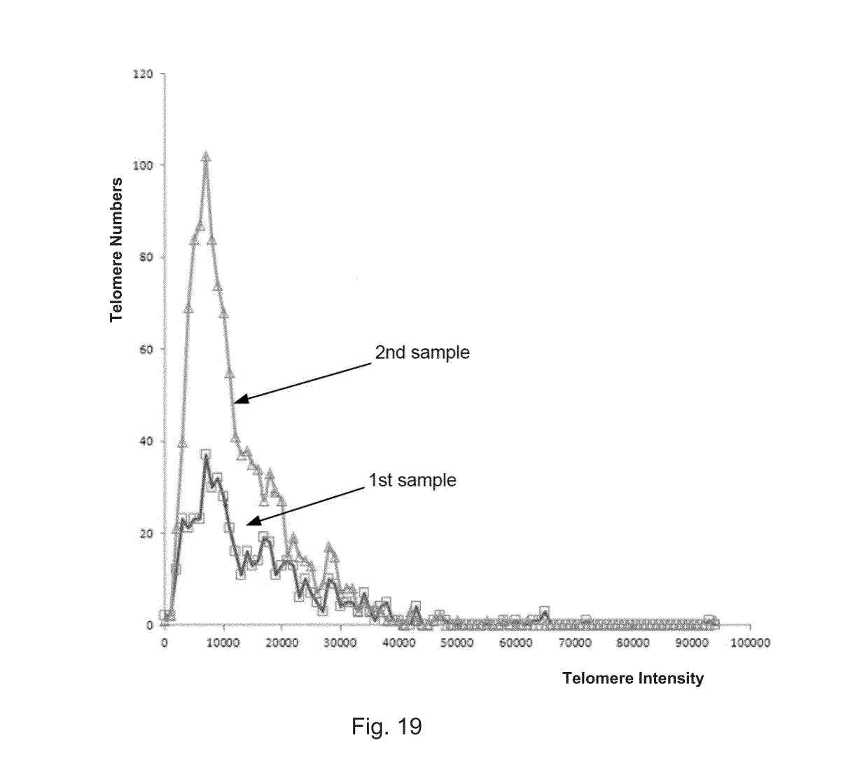

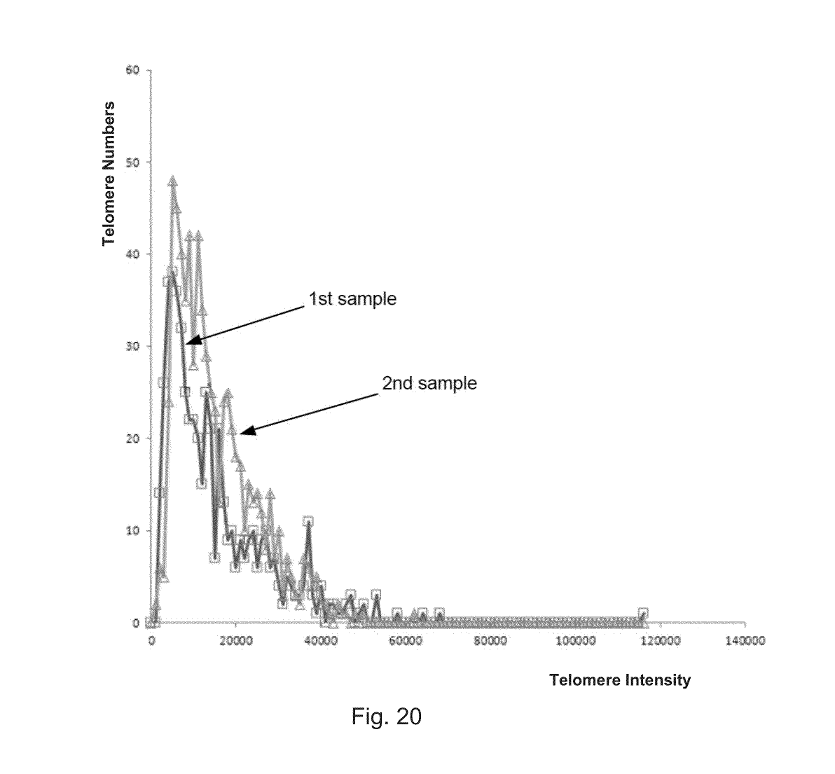

The stability of 3D telomere profiles of CTCs was assessed over a 6 month sampling period. Specifically, two blood samples were taken from a patient at an interval of about six months. The CTCs were isolated and analyzed as described in Examples 1&2. FIGS. 13-17 show that while the profiles were stable in some patients, they showed changes in others.

The patients assess are considered “intermediate risk group” and are monitored. The patients are not receiving treatment. No changes in the clinical appearance of the patients was detectable using current clinical tests (although it is predictable that some patients will progress. Some patients had the same CTC profile over the test period, whereas other patients had a differing CTC profile when compared to the baseline sample. CTC 3D profile changes may be detectable using the methods described herein before detection using current clinical tests. Further clinical data is being collected to ascertain when and/or whether changes in 3D profile manifest using current clinical tests. A longitudinal study is on going and will allow assessment of which subpopulations are associated with poor and good outcome.

Identification of different subpopulations now allows assessment of which population can be treated with a particular treatment regimen and/or which is associated with a better or worse outcome.

The CTC subpopulations isolated can be placed into cell culture and their response to treatment can be tested. NOTE we are the first to report these subpopulations based on telomeres, so this is a very new and open field.

### Example 5

### 3D Nuclear Imaging of Telomeres and Quantitative 3D Image Analysis of CTCs from a Large Cohort of Prostate Cancer Patients

### Summary

CTCs are isolated from the blood of prostate cancer patients who presented with positive biopsies that fall into three groups (low risk, intermediate risk and high-risk as determined by Gleason score). CTCs are isolated as described (Desitter et al, 2011), counted and imaged as outlined below. The specific 3D telomeric profiles found in prostate cancer CTCs are different from those found in normal cells and enable the identification of CTC sub-populations. Tissue biopsies from the same patients are examined for their 3D nuclear telomeric profiles and results compared to the data obtained with isolated CTCs.

### Methodology

Patients: Prostate cancer patients who consented to the study come from the Prostate Cancer Centre at CancerCare Manitoba. The patient cohort has includes patients who have not received prior treatments. The Prostate Centre performs on average 800 biopsies per year, of which 500 biopsies are positive. Within these 500 biopsies, ⅕ represents high-risk disease, while ⅖ are intermediate and low risk disease respectively. Two hundred patients falling into each of the three groups are examined in a blinded fashion. Two hundred patients with negative biopsies serve as controls.

CTC collection, biopsies and 3D telomere analysis: 7.5 ml of blood from prostate cancer patients who have not received prior treatments is received from the prostate cancer centre at CancerCare Manitoba. CTCs present in the blood sample are isolated using a filter device (Desitter et al., 2011). The 3D nuclear organization of the telomeres within the nuclei of captured cells is analyzed as described in Example 1.

Statistical analysis: Chi-square is used to compare the low/high CTC numbers per groups of patients. Applying a threshold of <5 CTCs/10⁹ blood cells as a marker for good/stable disease and >5 CTCs/10⁹ blood cells for poor/aggressive disease (Danila et al., 2010) establishes two groups of patients in the blindly analyzed samples; those with good/stable disease and those with poor/aggressive disease. 0.05 significance differences between low and high CTC numbers with 100 patients/group in the study are detected with a power of at least 80%.

### Conclusion

The information obtained during the study links CTCs with the clinical patient data. The 3D telomeric profiles in CTCs predict disease type.

### Example 6

Cells isolated using a filter device such as ScreenCell are analysed to identify subpopulations. The subpopulations can be isolated for example using microdissection for further analysis. For example, the cells can be subjected to PCR analysis, or probed using immunohistochemistry for example for the presence of tumour associated antigens etc, further tumour characterization (e.g. Her-2/neu detection).

Clusters of CTCs can be detected and compared for example to clusters of cells in the tumour. Cells can be stained and telomere organization analysis can be performed on the CTCs and correlated to the primary tumour (e.g. in terms of aggressiveness etc).

### Example 7

The CTC type that is most aggressive based on 3D provide will be determined using longitudinal studies.

Patients blood samples will be examined every six months over a period of minimum 3 years. Three years provides a sufficient time for ‘biochemical evidence for disease progression’ in prostate cancer.

Patients which progress or do not progress will be analysed for association with different CTC subpopulations.

All our analyses are done blinded.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Berthold D R, Pond G R, de Wit R, Eisenberger M, Tannock I F; TAX 327 Investigators. Survival and PSA response of patients in the TAX 327 study who crossed over to receive docetaxel after mitoxantrone or vice versa. Ann Oncol. 2008 October; 19(10):1749-53. Epub 2008 May 16.
2. Bubley G J, Carducci M, Dahut W, Dawson N, Daliani D, Eisenberger M, Figg W D, Freidlin B, Halabi S, Nudes G, Hussain M, Kaplan R, Myers C, Oh W, Petrylak D P, Reed E, Roth B, Sartor O, Scher H, Simons J, Sinibaldi V, Small E J, Smith M R, Trump D L, Wilding G, et al. Eligibility and response guidelines for phase II clinical trials in androgen-independent prostate cancer: recommendations from the Prostate-Specific Antigen Working Group. J Clin Oncol. 1999 November; 17(11):3461-7. Erratum in: J Clin Oncol. 2007 Mar. 20; 25(9)1154. J Clin Oncol 2000 July; 18(13):2644.
3. Budd G T, Cristofanilli M, Ellis M J, Stopeck A, Borden E, Miller M C, Matera J, Repollet M, Doyle G V, Terstappen L W M M, et al. Circulating tumor cells versus imaging-predicting overall survival in metastatic breast cancer. Clin Cancer Res. 2006; 12:6403.
4. Chuang T C, Moshir S, Garini Y, Chuang A Y, Young I T, Vermolen B, van den Doel R, Mougey V, Perrin M, Braun M, et al. The three-dimensional organization of telomeres in the nucleus of mammalian cells. BMC Biol. 2004; 2:12.
5. Cristofanilli M, Budd G T, Ellis M J, Stopeck A, Matera J, Miller M C, Reuben J M, Doyle G V, Allard W J, Terstappen L W, Hayes D F. Circulating tumor cells, disease progression, and survival in metastatic breast cancer. N Engl J Med. 2004 Aug. 19; 351(8):781-91.
6. Danila D C, Fleisher M, Scher H I. Circulating tumor cells as biomarkers in prostate cancer. Clin Cancer Res. 2011 Jun. 15; 17(12):3903-12.
7. de Lange T. Shelterin: the protein complex that shapes and safeguards human telomeres. Genes Dev. 2005 Sep. 15; 19(18):2100-10.
8. De Vos W H, Hoebe R A, Joss G H, Haffmans W, Baatout S, Van Oostveldt P, Manders E M. Controlled light exposure microscopy reveals dynamic telomere microterritories throughout the cell cycle. Cytometry A. 2009; 75A:428-439.
9. Deng Y, Chan S S, Chang S Telomere dysfunction and tumour suppression: the senescence connection. Nat Rev Cancer. 2008 June; 8(6):450-8.
10. DePinho R A, Polyak K. Cancer chromosomes in crisis. Nat Genet. 2004 September; 36(9):932-4.
11. Goodman O B Jr, Fink L M, Symanowski J T, Wong B, Grobaski B, Pomerantz D, Ma Y, Ward D C, Vogelzang N J. Circulating tumor cells in patients with castration-resistant prostate cancer baseline values and correlation with prognostic factors. Cancer Epidemiol Biomarkers Prey. 2009 June; 18(6):1904-13.
12. Desitter I, Guerrouahen B S, Benali-Furet N, Wechsler J, Janne P A, Kuang Y, Yanagita M, Wang L, Berkowitz J A, Distel R J, Cayre Y E. A new device for rapid isolation by size and characterization of rare circulating tumor cells. Anticancer Res. 2011 February; 31(2):427-41.
13. Doyen J, Alix-Panabières C, Hofman P, Parks S K, Chamorey E, Naman H, Hannoun-Lévi J M. Circulating tumor cells in prostate cancer: A potential surrogate marker of survival. Crit Rev Oncol Hematol. 2011 Jun. 14. [Epub ahead of print]
14. Fleming M T, Morris M J, Heller G, Scher H I. Post-therapy changes in PSA as an outcome measure in prostate cancer clinical trials. Nat Clin Pract Oncol. 2006 December; 3(12):658-67. Review.
15. Gadji M, Fortin D, Tsanaclis A M C, Yuval G, Katzir N, Wienburg Y, Yan J, Klewes L, Klonisch T, Drouin R, Mai S. Three-dimensional (3D) nuclear telomere architecture is associated with differential time to progression and overall survival in glioblastoma patients. Neoplasia (2): 183-191 2010.
16. Gadji M, Awe J A, Rodriguez P, Klewes L, Kumar R, Houston D S, Falcao R P, de Oliveira F M, Mai S. Three-dimensional nuclear telomeric architecture defines cytopenias of myelodysplastic syndrome and its transformation to acute myeloid leukemia. Clin Cancer Res. 2012; 18:3293.
17. Glaves D. Correlation between circulating tumor cancer cells and incidence of metastases. Br J. Cancer. 1984; 50:159-166.
18. Gonzalez-Suarez I, Redwood A B, Perkins S M, Vermolen B, Lichtensztejin D, Grotsky D A, Morgado-Palcin L, Gapud E J, Sleckman B P, Sulivan T, Sage J, Steward C L, Mai S, Gonzalo S (2009) Novel roles for A type lamins in telomere biology and DNA damage response pathway EMBO J 28, 2414-27.
19. Gulley J L, Drake C G. Immunotherapy for prostate cancer: recent advances, lessons learned, and areas for further research. Clin Cancer Res. 2011 Jun. 15; 17(12):3884-91.
20. Kane R D, Hawkins H K, Miller J A, Noce P S. Microscopic pulmonary tumor emboli associated with dyspnea. Cancer. 1975; 36:1473-1482.
21. Klewes L, Hobsch C, Katzir N, Rourke D, Garini Y, Mai S, Novel automated three-dimensional genome scanning based on the nuclear architecture of telomeres. Cytometry A. 2011 February; 79(2):159-66. doi: 10.1002/cyto.a.21012. Epub 2010 Dec. 30.
22. Knecht H, Sawan B, Lichtensztejn D, Lemieux B, Wellinger R J, Mai S. The 3D nuclear organization of telomeres marks the transition from Hodgkin to Reed-Sternberg cells. Leukemia. 2009 March; 23(3):565-73.
23. Knecht H, Sawan B, Lichtensztejn Z, Lichtensztejn D, Mai S. 3D telomere FISH defines LMP1 expressing Reed-Sternberg cells as end-stage cells with telomere-poor “ghost” nuclei. Lab Invest. 2010; 90(4):611-619
24. Lansdorp P M. Telomeres and disease. EMBO J. 2009 Sep. 2; 28(17):2532-40. Epub 2009 Jul. 23. Review.
25. Louis S F, Vermolen B J, Garini Y, Young I T, Guffei A, Lichtensztejn Z, Kuttler F, Chuang T C, Moshir S, Mougey V, Chuang A Y, Kerr P D, Fest T, Boukamp P, Mai S. c-Myc induces chromosomal rearrangements through telomere and chromosome remodeling in the interphase nucleus. Proc Natl Acad Sci USA. 2005 Jul. 5; 102(27):9613-8.
26. Madan R A, Pal S K, Sartor O, Dahut W L. Overcoming chemotherapy resistance in prostate cancer. Clin Cancer Res. 2011 Jun. 15; 17(12):3892-902.
27. Mai S, Garini Y. Oncogenic remodeling of the three-dimensional organization of the interphase nucleus: c-Myc induces telomeric aggregates whose formation precedes chromosomal rearrangements. Cell Cycle. 2005 October; 4(10):1327-31.
28. Mai S, Garini Y. The significance of telomeric aggregates in the interphase nuclei of tumor cells. J Cell Biochem. 2006 Apr. 1; 97(5):904-15.
29. Mai S. Initiation of telomere-mediated chromosomal rearrangements in cancer. J Cell Biochem. 2010 Apr. 15; 109(6):1095-102.
30. Meeker A K, Hicks J L, Gabrielson E, Strauss W M, De Marzo A M, Argani P. Telomere shortening occurs in subsets of normal breast epithelium as well as in situ and invasive carcinoma. Am J Pathol. 2004 March; 164(3):925-35.
31. Misri S, Pandita S, Kumar R, Pandita T K. Telomeres, histone code, and DNA damage response. Cytogenet Genome Res. 2008; 122(3-4):297-307. Epub 2009 Jan. 30. Review.
32. Molnar B, Floro L, Sipos F, Toth B, Sreter L, Tulassay Z. Circulating tumor cell clusters in the peripheral blood of colorectal cancer patients. Clin Cancer Res. 2001; 7:4080.
33. Murnane J P, Sabatier L. Chromosome rearrangements resulting from telomere dysfunction and their role in cancer. Bioessays. 2004 November; 26(11):1164-74.
34. Sarkar R., Guffei A, Vermolen B J, Garini Y, Mai S. Alterations of centromere positions in nuclei of immortalized and malignant mouse lymphocytes. Cytometry A. 2007; 71(6):386-392
35. Schaefer L H, Schuster D, Herz H. Generalized approach for accelerated maximum likelihood based image restoration applied to three-dimensional fluorescence microscopy. J Microsc. 2001 November; 204(Pt 2):99-107.
36. Scher H I, Jia X, de Bono J S, Fleisher M, Pienta K J, Raghavan D, Heller G. Circulating tumour cells as prognostic markers in progressive, castration-resistant prostate cancer: a reanalysis of IMMC38 trial data. Lancet Oncol. 2009 March; 10(3):233-9. Epub 2009 Feb. 11.
37. Scher H I, Morris M J, Kelly W K, Schwartz L H, Heller G. Prostate cancer clinical trial end points: “RECIST”ing a step backwards. Clin Cancer Res. 2005 Jul. 15; 11(14):5223-32. Review.
38. Scher H I, Halabi S, Tannock I, Morris M, Sternberg C N, Carducci M A, Eisenberger M A, Higano C, Bubley G J, Dreicer R, Petrylak D, Kantoff P, Basch E, Kelly W K, Figg W D, Small E J, Beer T M, Wilding G, Martin A, Hussain M; Prostate Cancer Clinical Trials Working Group. Design and end points of clinical trials for patients with progressive prostate cancer and castrate levels of testosterone: recommendations of the Prostate Cancer Clinical Trials Working Group. J Clin Oncol. 2008 Mar. 1; 26(7):1148-59.
39. Schwarzenbach H, Hoon D S, Pantel K. Cell-free nucleic acids as biomarkers in cancer patients. Nat Rev Cancer. 2011 June; 11(6):426-37. Epub 2011 May 12. Review.
40. Selvarajah S, Yoshimoto M, Park P C, Maire G, Paderova J, Bayani J, Lim G, Al-Romaih K, Squire JA, Zielenska M. The breakage-fusion-bridge (BFB) cycle as a mechanism for generating genetic heterogeneity in osteosarcoma. Chromosoma. 2006 December; 115(6):459-67. Epub 2006 Aug. 9.
41. Stewénius Y, Gorunova L, Jonson T, Larsson N, Hoglund M, Mandahl N, Mertens F, Mitelman F, Gisselsson D. Structural and numerical chromosome changes in colon cancer develop through telomere-mediated anaphase bridges, not through mitotic multipolarity. Proc Natl Acad Sci USA. 2005 Apr. 12; 102(15):5541-6. Epub 2005 Apr. 4.
42. Vermolen B J, Garini Y, Mai S, Mougey V, Fest T, Chuang T C, Chuang A Y, Wark L, Young I T. Characterizing the three-dimensional organization of telomeres. Cytometry A. 2005 October; 67(2):144-50.
43. Vukovic B, Park P C, Al-Maghrabi J, Beheshti B, Sweet J, Evans A, Trachtenberg J, Squire J. Evidence of multifocality of telomere erosion in high-grade prostatic intraepithelial neoplasia (HPIN) and concurrent carcinoma. Oncogene. 2003 Apr. 3; 22(13):1978-87.
44. Vukovic B, Beheshti B, Park P, Lim G, Bayani J, Zielenska M, Squire J A. Correlating breakage fusion-bridge events with the overall chromosomal instability and in vitro karyotype evolution in prostate cancer. Cytogenet Genome Res. 2007; 116(1-2):1-11.
45. Zheng S, Lin H, Liu J Q, Balic M, Datar R, Cote R J, Tai Y C. Membrane microfilter device for selective capture, electrolysis and genomic analysis of human circulating tumor cells. J Chromatogr A. 2007 Aug. 31; 1162(2):154-61. Epub 2007 May 29.

1. A method of identifying one or more circulating tumour cell (CTC) subpopulations comprising:
a. isolating CTCs from a blood sample from a subject;
b. determining the 3D telomere organization signature of each of a plurality of the isolated CTCs; and
c. identifying one or more sub-populations of the CTCs based on one or more of 3D telomere organization signature features selected from telomere number, telomere size, presence and/or number of telomeric aggregates, telomeres per nuclear volume, distances from nuclear centre and a/c ratio.

2. The method of claim 1, wherein the CTCs are isolated from the blood sample using a filter.

3. The method of claim 1, wherein the CTCs are from a subject with prostate cancer, melanoma, breast cancer, colon cancer or lung cancer.

4-5. (canceled)

6. The method of claim 1, wherein the method comprises identifying:
i. a first sub-population comprising CTCs with an average telomere intensity of less than about 20,000, less than about 15,000, less than about 10,000 or less than about 5,000 a.u.;
ii. a second sub-population comprising CTCs with an average telomere intensity of about 5,000-10,000 to about 20,000-50,000 a.u.; and/or
iii. a third sub-population comprising CTCs with an average telomere intensity of more than about 20,000, more than about 25,000, more than about 30,000, more than about 40,000 or more than about 50,000 a.u.; or
wherein the method comprises identifying:
i. a first sub-population comprising CTCs with an average telomere intensity of less than about 20,000, less than about 25,000, less than about 30,000, less than about 35,000 or less than about 40,000 a.u.; and/or
ii. a second sub-population comprising CTCs with an average telomere intensity of more than about 20,000, more than about 25,000, more than about 30,000, more than about 35,000 or more than about 40,000 a.u.

7. (canceled)

8. The method of claim 1, wherein the method further comprises isolating the sub-population identified in step (c).

9. A method for identifying CTC subpopulations, the method comprising:
a. obtaining a plurality of 3D telomere organization signature datasets, each dataset corresponding to a unique isolated CTC;
b. determining for each dataset, values for features from the 3D telomere organization signature datasets; and
c. determining the number of subpopulations based on a combination of the values of the features;
wherein the features comprise at least one of telomere number, telomere size, presence and/or number of telomeric aggregates, telomeres per nuclear volume, distances from nuclear centre and a/c ratio.

10-12. (canceled)

13. The method of claim 9, wherein the number of subpopulations is assessed, by comparing one or more of telomere numbers, sizes, nuclear volumes, telomere distribution within the nucleus and/or nuclear sizes.

14-15. (canceled)

16. An isolated sub-population of circulating tumour cells (CTCs) obtained by:
isolating one or more of the sub-populations identified in claim 1.

17. The isolated sub-population of claim 16, wherein the sub-population comprises CTCs with an average telomere intensity of less than about 20,000, less than about 15,000, less than about 10,000 or less than about 5,000 a.u.; optionally wherein the sub-population comprises CTCs with an average telomere intensity of about 5,000-10,000 to about 20,000-50,000 a.u.; optionally wherein the sub-population comprises CTCs with an average telomere intensity of more than about 20,000, more than about 25,000, more than about 30,000, more than about 40,000 or more than about 50,000 a.u.; optionally wherein the sub-population comprises CTCs with an average telomere intensity of more than about 20,000, more than about 25,000, more than about 30,000, more than about 35,000 or more than about 40,000 a.u. or less than about 20,000, less than about 25,000, less than about 30,000, less than about 35,000 or less than about 40,000 a.u.

18-20. (canceled)

21. An assay comprising:
a. determining a 3D telomeres organization signature for a plurality of isolated test CTCs isolated from a blood sample from a subject with cancer;
b. identifying one or more subpopulations according to a method of claim 1; and
c. comparing the 3D telomeres organization signature of the test CTC subpopulations with a reference 3D telomeres organization signature, and if there is a difference or similarity in the 3D telomeres organization signature of the test CTCs and the reference 3D telomeres organization signature, identifying the subject as having an increased probability of a positive or negative clinical outcome.
wherein the clinical outcome is progression or recurrence;
wherein the 3D telomeres organization signature comprises one or more of telomere number, telomere size, presence and/or number of telomeric aggregates, telomeres per nuclear volume, distances from nuclear centre and a/c ratio.

22-24. (canceled)

25. The assay of claim 21 wherein the 3D telomeres organization signature comprises one or more of telomere numbers, telomere size and number of aggregates, and
wherein an aberrant number of telomeres, a decrease in average telomere size and/or an increased number of aggregates in the 3D telomeres organization signature of the test CTCs is indicative of an increased probability of a negative clinical outcome.

26. The assay of claim 21 wherein the presence of telomere aggregates in at least 35%, 40%, 45%, 50%, 55%, 60%, 70% or 80% of the test CTCs is indicative of an increased probability of a negative clinical outcome and/or wherein the population of test CTCs is organized into sub-populations based on telomere size and more than 2, 3, 4 or 5 sub-populations is indicative of an increased probability of a negative clinical outcome.

27. The assay of claim 21, wherein the assay further comprises identifying the number of CTCs in the blood sample and wherein more than about 25, more than about 30, more than about 35, more than about 40, more than about 45, more than about 50, more than about 60, more than about 70 or more than about 80 CTCs in 3.5 mL of blood is indicative of an increased probability of a negative clinical outcome.

28. (canceled)

29. A method of prognosing a clinical outcome in a subject with cancer comprising:
a. isolating CTCs from a blood sample from the subject to obtaining test sample CTCs using a filter device, and
b. determining a 3D telomere organization signature of the test sample CTCs using 3D q-FISH;
wherein the 3D telomere organization signature comprises one or more of telomere number, telomere size, presence and/or number of telomeric aggregates, telomeres per nuclear volume, distances from nuclear centre and a/c ratio; and
wherein the 3D telomere organization signature of the test sample CTCs is indicative of the clinical outcome of the subject; optionally wherein the clinical outcome is progression or recurrence.

30-32. (canceled)

33. The method of claim 29, further comprising step c), comparing the 3D telomere organization signature of the test sample CTCs with a 3D telomere organization signature in a control, wherein a difference or similarity in the 3D telomere organization signature between the test sample CTCs and the control is indicative of the clinical outcome of the subject.

34. The method of claim 29, wherein the cancer is melanoma, colorectal cancer, lung cancer, breast cancer or prostate cancer.

35. (canceled)

36. The method of claim 29 wherein the 3D telomeres organization signature comprises one or more of telomere numbers, telomere size and number of aggregates,
and wherein an aberrant number of telomere, a decrease in average telomere size and/or an increased number of aggregates in the 3D telomeres organization signature of the test CTCs is indicative of an increased probability of a negative clinical outcome.

37. The method of claim 29, wherein the presence of telomere aggregates in at least 35%, 40%, 45%, 50%, 55%, 60%, 70% or 80% of the test CTCs is indicative of an increased probability of a negative clinical outcome; and/or wherein population of test CTCs is organized into sub-populations based on telomere size and more than 2, 3, 4 or 5 sub-populations is indicative of an increased probability of a negative clinical outcome.

38. The method of claim 29, wherein the assay further comprises identifying the number of CTCs in the blood sample and wherein more than about 25, more than about 30, more than about 35, more than about 40, more than about 45, more than about 50, more than about 60, more than about 70 or more than about 80 CTCs in 3.5 mL of blood is indicative of an increased probability of a negative clinical outcome.

39. (canceled)

40. A method of treating a subject, comprising prognosing the clinical outcome of a subject according to the method of claim 29 and providing a suitable treatment according to the prognosis.
